(19) United States
(12) Patent Application Publication
(10) Pub. No.: US 2024/0002809 A1
(43) Pub. Date: Jan. 4, 2024

(54) FRACTAL CUES SUPPORT HIERARCHICAL MATURATION OF CELLS VIA CURVATURE-INDUCED PATTERNING

(71) Applicants: Anastasia Korolj, Toronto (CA); Milica Radisic, Toronto (CA); Ana Konvalinka, Toronto (CA); Chuan Liu, Toronto (CA); The Governing Council of the University of Toronto, Toronto (CA)

(72) Inventors: Anastasia KOROLJ, Toronto (CA); Milica RADISIC, Toronto (CA); Ana KONVALINKA, Toronto (CA); Chuan LIU, Toronto (CA)

(21) Appl. No.: 18/216,504

(22) Filed: Jun. 29, 2023

Related U.S. Application Data
(60) Provisional application No. 63/356,948, filed on Jun. 29, 2022.
(60) Provisional application No. 63/477,715, filed on Dec. 29, 2022.

(51) Int. Cl. C12N 5/071; C12M 1/12; C12M 3/06; C12M 3/00; C12M 1/34

(52) U.S. Cl. CPC C12N 5/0686; C12M 25/14; C12M 23/16; C12M 21/08; C12M 41/46; C12N 2513/00

(57) ABSTRACT

An apparatus, a method for fabricating the apparatus, and a method for cultivation of cells are provided. The apparatus comprises a first chamber for cultivating cells and a surface, supported in the first chamber, for cell cultivation thereon. The surface exhibits one or more fractal features, each fractal feature comprising out-of-plane fractal patterning providing non-planar microtopology for the surface.

### CROSS-REFERENCE TO RELATED APPLICATIONS

The present disclosure claims priority from U.S. provisional patent application No. 63/356,948, entitled “FRACTAL CUES SUPPORT HIERARCHICAL MATURATION OF PODOCYTES VIA CURVATURE-INDUCED PATTERNING”, filed Jun. 29, 2022, and from U.S. provisional patent application No. 63/477,715, entitled “FRACTAL CUES SUPPORT HIERARCHICAL MATURATION OF PODOCYTES VIA CURVATURE-INDUCED PATTERNING”, filed Dec. 29, 2022, the entireties of which are hereby incorporated by reference.

### FIELD

The present disclosure relates to cultivation of biological cells. In particular, the present disclosure relates to three-dimensional structure with fractal patterning for promoting high-fidelity podocyte cultivation.

### BACKGROUND

Complex tissues operate in three-dimensional space, yet standard cell culture methods are faced with dimension reduction, e.g., well plates designed for monolayer culture and analysis.

For example, podocytes are terminally differentiated epithelial cells that wrap around glomerular capillaries and exhibit bifurcating processes responsible for sieve-like barrier function during blood purification. Podocytes increase their structural complexity as capillaries loop from flat to bundled geometries during development⁴. Indeed, reduction of podocyte branching (known as foot process effacement) is a marker of dysfunction, and a pathological finding in many kidney diseases and injuries⁵. Once lost, podocytes are not known to regenerate. Their loss results in glomerular kidney disease, marked by significant proteinuria. Apart from proteinuria and impairment of glomerular filtration, there is a paucity of specific podocyte injury biomarkers, resulting in the lack of precise diagnostic tools and a paucity of specific treatments. Since animal models do not fully recapitulate human kidney physiology and pathophysiology, new lab-based preclinical approaches such as organ-on-a-chip systems are needed to accelerate kidney research. However, recapitulating physiologically relevant podocytes in culture remains a challenge⁶. Gene expression reaches plateaus far from the native conditions, phenotypes are persistently immature, and comprehensive readouts that quantify podocyte function are lacking. While factors including biochemical differentiation⁷, organoid growth⁸, shear stress from fluid flow^{9,10}, mechanical stimulation by stretching¹¹, tuning of substrate stiffness¹², and 3D co-culture¹³ have demonstrated notable advances in podocyte maturation, mimicking the podocyte and similar microenvironments has not yet been achieved.

Most cell and tissue culture approaches rely on flat cultivation substrates for growing cells, including flat membranes in microfluidic systems. Although it has been observed that simple micro-curvature may support higher-fidelity podocyte culture in vitro¹⁴, mechanisms explaining why patterned micron-scale shapes may facilitate maturation are still not understood. As a result, shape stimulation is not widely used. Similarly, while a goal of organ-on-a-chip systems is to increase in vitro complexity, the role of complexity itself has not yet been investigated. Establishment of complex branching morphology and sensitive podocyte responses to stimuli have also not yet been achieved nor previously been measurable in vitro.

### SUMMARY

Nature efficiently self-organizes cells and tissues into complex fractal forms. Fractal microcurvature was associated with charge density gradients, and organized extracellular matrix that led to the assembly of cell structures and hierarchical cell branching in vitro, delineated using a novel fluorescent assaying technique. Fractally stimulated cells reflected biological complexity more completely than flat cultures in applications of drug testing, coronavirus infection, and a cells-as-sensors approach to patient serum diagnostics, thus setting a precedent for how fractal frameworks may support higher-fidelity bioengineering.

The present disclosure describes examples of biomimetic, microfabricated platforms having three-dimensional fractal features to induce functional differentiation of various cell types, including epithelial podocytes, a cell type found in the kidney nephron. The present disclosure may also be useful for any other cells that respond favorably to a fractal cultivation surface. For example, bronchial epithelial cells, blood vessels, lung cells, bone marrow, or any other tissue having a fractal topology may be cultivated using the disclosed methods and apparatuses.

In some examples, the present disclosure describes an apparatus for cultivation of cells. The apparatus includes a first chamber for cultivating cells; and a surface, supported in the first chamber, for cell cultivation thereon, the surface exhibiting one or more fractal features, each fractal feature comprising out-of-plane fractal patterning providing non-planar microtopology for the surface.

In some examples, the present disclosure describes a method for cultivating cells. The method providing an apparatus comprising: a first chamber for cultivating cells; and a surface, supported in the first chamber, for cell cultivation thereon, the surface exhibiting one or more fractal features, each fractal feature comprising out-of-plane fractal patterning providing non-planar microtopology for the surface; introducing cells onto the surface of the apparatus; and promoting differentiation of the cells.

In some aspects, the present disclosure provides a method for fabricating an apparatus for cultivation of cells, the method comprising: drawing fractal patterning onto a substrate using photolithography; generating non-planar microtopology on the substrate according to the fractal patterning; forming an inverse mold by curing a first polymer over the non-planar microtopology with fractal patterning; forming a surface for cell cultivation by curing a second polymer using the inverse mold, the surface being formed to exhibit one or more fractal features, each fractal feature comprising the out-of-plane fractal patterning providing non-planar microtopology for the surface; and supporting at least a portion of the surface in a first chamber for cultivating cells.

In some examples of any of the preceding aspects/embodiments, each fractal feature includes protruding Gaussian, fractal micro-curvatures.

In some examples of any of the preceding aspects/embodiments, the fractal patterning is derived from a histological section of a biological microenvironment exhibiting the fractal patterning.

In some examples of any of the preceding aspects/embodiments, the biological microenvironment is a podocyte microenvironment, where the fractal patterning mimics glomeruli in the podocyte microenvironment, and the micro-curvatures are in convoluted capillary shapes.

In some aspects, the present disclosure provides a method of viewing confluent branching cell morphology of cells, the method comprising: sporadically labeling cytoplasm of a portion of the cells with green fluorescent protein (GFP) in vitro; mixing the GFP-labelled cells with another non-fluorescent portion of the cells; staining the mixed cells to visualize confluent monolayers; identifying single GFP-labelled cells in contact with non-fluorescent cells that are arranged in a monolayer; and imaging the monolayer.

### BRIEF DESCRIPTION OF THE DRAWINGS

Reference will now be made, by way of example, to the accompanying drawings which show example embodiments of the present application, and in which:

FIG. 1a shows that a fractal space-filling curve has a dimension in between a 1.0-D line and 2.0-D plane, and a porous material is a fractal in between a 2.0-D plane and a 3.0-D cube;

FIG. 1b shows a block-face scan of a single podocyte cell reproduced with permission from⁴⁵, and cast of a glomerulus reproduced with permission from⁴⁶;

FIG. 1c shows in vivo capillary and podocyte development from flat and cobblestone-like to rounded and fractal. Obtained with permission^{S1};

FIG. 1d illustrates outlining and quantification of in vivo podocyte fractal morphology during development using n=3-10 outlined cells per group. Obtained with permission^{S1};

FIG. 1e illustrates outlining and fractal dimension quantification of in vivo structured illumination micrographs for nephrin-stained control and minimal change disease podocytes, from n=1 available image each. Obtained with permission^{S2};

FIG. 1f illustrates outlining and quantification of in vivo wild-type and vinculin knock-out podocytes, from n=1 whole cells or n=2 foot process sub-sections. Obtained with permission^{S3}. Data shown are average±SD. Analysis by one-way ANOVA and Fisher's LSD multiple comparisons test, p≤0.05 considered significant. MCD=minimal change disease. Df=fractal dimension.

FIG. 1g is an illustration of fractal vs. non-fractal object and their differential scaling complexity;

FIG. 1h is a schematic, cross-sectional H&E histology, and projected traces of kidney glomeruli patterns, taken from healthy and pathological (proliferative lesions) sections. Histology reproduced with permission¹⁶;

FIGS. 1i and 1j show fractal dimension and perimeter quantification, respectively, of glomeruli traces from FIG. 1h. T-test with significance level of 5% was performed on n=3 images per group. Df is significant to two decimal places;

FIG. 1k shows LAMC1-stained glomeruli histology sections from patient biopsies from antibody mediated rejection, scored by pathologist;

FIG. 2a is a flow diagram of steps involved in fabrication of biomimetic 2½-D miCRAFT scaffolds for cell culture. Glomerulus cast reproduced with permission from⁴⁶ and histology from⁴¹;

FIG. 2b illustrates 2D design and 3D profilometer-reconstructed patterns for non-topographic (NT), round non-fractal (RT), and biomimetic low- and high-dose fractal scaffolds (LF and HF);

FIG. 2c illustrates quantification of scaffolds of FIG. 2b based on slice diameter and feature height distribution;

FIG. 2d illustrates fractal dimension, and surface area-to-volume ratio of scaffolds of FIG. 2b, where N=3 patterns quantified;

FIG. 2e shows scanning electron micrographs (SEM) of scaffolds of FIG. 2b and second harmonic generation (SHG) micrographs of collagen I coating on the scaffolds, showing molecular arrangement according to scaffold pattern. Low magnification (top row) and high magnification (bottom row) shown;

FIG. 2f shows a Zeta potential-pH curve for PDMS with topography;

FIG. 2g is Kelvin probe force microscopy (KPFM) images showing microscale curvature-potential correlation for 20 μm×20 μm sections including flat and curved region of fractal scaffolds of FIG. 2b;

FIG. 2h are Zeta potential measurements for scaffolds of FIG. 2b at neutral pH, n=3 scaffolds measured n=3-5 times;

FIG. 2i are schematics of charge density on curved surfaces of the scaffolds of FIG. 2b. Data shown as average±SD analyzed with one-way ANOVA using Fisher's LSD test for multiple comparisons. p≤0.05 considered significant. NT=No Topography; RT=Round Topography; LF=Low Fractal; HF=High Fractal;

FIG. 3a illustrate fractal topographic patterning supports hierarchical assembly of cellular machinery at multiple scales in mouse podocytes, specifically schematics of cellular components involved in polarity for reader orientation;

FIG. 3b illustrates mouse podocytes confocal images with scaffold ‘slice’ outlined in white hexagons;

FIG. 3c illustrates quantification of cell number per image of FIG. 3b;

FIGS. 3d(1) and (2) shows immunofluorescent staining of proteins involved in podocyte development, function, and structural organization;

FIG. 3e illustrates quantification of immunofluorescence signal expression (raw integrated density) of FIG. 3b;

FIG. 3f shows localization of protein in cytoplasmic regions of FIG. 3b, as ratio of cytoplasmic/nuclear expression levels. Immunofluorescence expression quantification was normalized by cell number. Data are shown as average±SD and performed on n=6 scaffold samples. One-way ANOVA statistical testing, using Fisher's LSD test for multiple comparisons. p≤0.05 considered significant;

FIG. 3g shows immunofluorescent staining of cellular machinery at multiple scales in human podocyte cell line, specifically schematic of cellular components involved in polarity for reader orientation;

FIG. 3h are human podocytes confocal images with scaffold ‘slice’ outlined in white hexagons;

FIG. 3i illustrates quantification of cell number per image in FIG. 3h;

FIGS. 3j(1), (2), and (3) show immunofluorescent staining of proteins in FIG. 3h involved in podocyte development, function, and structural organization

FIG. 3k illustrates quantification of immunofluorescence signal expression (raw integrated density);

FIG. 3l show localization of protein in cytoplasmic regions of FIG. 3h, as ratio of cytoplasmic/nuclear expression levels. Immunofluorescence expression quantification was normalized by cell number. Data are shown as average±SD and performed on n=9 scaffold samples. One-way ANOVA statistical testing, using Fisher's LSD test for multiple comparisons. p≤0.05 considered significant;

FIG. 3m are a podocalyxin immunofluorescent images with pattern section enlarged, with corresponding SEM of scaffold slice pattern in mouse cell line;

FIG. 3n are an integrin ß-1 immunofluorescent stain images with localized enrichment of signal in mouse cell line. N=1 scaffold;

FIGS. 3o and 3p show live/dead staining and quantification of human podocyte cell line attachment to various ECM matrices. Collagen I, mixture of 50% collagen I and laminin 511, laminin 511, Matrigel, porcine kidney-derived NativeCoat ECM, and no coating control, on PDMS flat surfaces assessed three hours post-seeding. N=8 scaffold samples. Data are shown as average±SD. Analyzed by one-way ANOVA with Fisher's LSD multiple comparison test, p≤0.05 considered significant;

FIG. 4a shows a mosaic assay for mouse cell line cultivated in vitro for 3 days (top panel) and 14 days (lower panel). Inset shows detail on fractal branching of foot processes;

FIG. 4b illustrates quantification of whole cell morphology by fractal dimension and by FIG. 4c, counting degrees of branching, for mouse cell line in A;

FIG. 4d illustrates sampled regions showing process formation and interdigitation;

FIG. 4e illustrates quantified manually by: digitation ratio of perimeter around processes normalized by perpendicular boundary line; approximate density by number of processes per micrometer of boundary; and approximate length of processes determined by the perimeter around processes along the boundary line divided by two;

FIG. 4f is a schematic of manual digitation analysis, reproduced with permission5. Cells imaged after 14 days of differentiation culture. N=6 scaffolds. Data are shown as average±SD. Analyzed by one-way ANOVA with Fisher's LSD multiple comparison test, p≤0.05 considered significant;

FIG. 4g shows a mosaic assay for human cell line after 5 days cultivation with FIG. 4h illustrating quantification of whole cell morphology by fractal dimension and FIG. 4i, by counting degrees of branching;

FIG. 4j are examples of mouse cells detected by Mosaic assay;

FIG. 4k are examples of human cells detected by Mosaic assay;

FIG. 4l shows SEM of mouse podocyte apical surface with budding protrusions;

FIG. 4m shows NTA particle size and concentration distribution at day 4 and day 14 of mouse podocyte culture;

FIGS. 4n and 4o illustrate quantification of average particle sizes secreted at day 4 and day 14, respectively;

FIG. 4p shows results of Kolmogorov-Smirnov (K-S) test comparing NTA particle distribution patterns. Quantification in panels B-C, E-F performed on images from a total of n=9 scaffold samples per group, K-L on n=3 samples pooled from n=12 scaffold samples. Data are shown as average±SD. One- or two-way ANOVA statistical testing, using Fisher's LSD test for multiple comparisons. p≤0.05 considered significant. SEM=scanning electron micrograph. NTA=nanoparticle tracking analysis;

FIG. 5a illustrates an unsupervised principal component analysis (PCA) delineating clusters in total gene expression patterns for three main conditions NT, RT, H F;

FIG. 5b is a Venn diagram of differentially expressed genes (DEG) for comparisons between three main conditions;

FIG. 5c is a PluriTest showing increasing maturation down y-axis, compared to control groups from GSE116471⁷;

FIG. 5d is a k-means clustered heat map for podocyte identity genes revealing enriched DEG in ECM organization and development-related processes. Podocyte identity gene list from Park et al.³²;

FIG. 5e are Selected significant pathways enriched from FIG. 5d;

FIGS. 5f(1) and (2) are GO terms of pathways enriched in k-means cluster analyses, showing clustering of podocyte identity genes, based on list from Park et al.^{S6};

FIG. 5g shows clustering of subset of genes where topographic stimulation matches mature expression signature, based on data from Yoshimura et al.^{S7} n=4 scaffold samples per group, primary fetal human podocytes, after 3 days in culture;

FIGS. 5h(1)-(3) are volcano plots with annotated differentially expressed genes. Colour legends shows manual annotation of gene functions. N=4 scaffold samples per group, primary fetal human podocytes, after 3 days in culture.

FIG. 5i is a heat map for comprehensive ECM-associated gene list. N=4 scaffold samples per group, primary fetal human podocytes, after 3 days in culture;

FIG. 5j are PCA and heat maps for curated gene lists for Microtubule-binding, FIG. 5k for actin-binding, and FIG. 5l for integrin gene lists. n=4 scaffold samples per group, primary fetal human podocytes, after 3 days in culture;

FIG. 5m relates to Gene ontology biological compartments enriched, where boxes are labelled with top significant GO terms and associated p-values. Dashed boxes show compartments of interest. N=4 scaffold samples per group, primary fetal human podocytes, after 3 days in culture;

FIG. 5n shows a subset of genes where topographic cells match adult expression signature from GSE116471⁷;

FIG. 5o shows k-means clustering of FIG. 5n;

FIG. 5p shows selected significant pathways enriched from FIG. 5o. RNA sequencing was performed on n=4 sample replicates from one batch of primary GW18 fetal human podocytes after 3 days in culture with scaffolds;

FIG. 6a illustrates a mosaic assay response to model virus infection by human coronaviruses 229e and NL63 on human podocyte cell line;

FIG. 6b illustrates quantification of podocyte morphology 12 hours following model virus infection of either 10 minutes or 1 hour of incubation time on NT or HF scaffolds. N=9 samples for controls, n=3 samples at 10 minutes, and n=6 samples at 1 hour;

FIG. 6c shows a mosaic assay response to model virus infection by human coronaviruses 229e and NL63 on human podocyte cell line at 24 hours post-infection, cultivated on miCRAFT;

FIG. 6d illustrates quantification of cell morphology 24 and 48 hours following model virus infection of either 10 minutes or 1 hour of incubation time on NT or HF scaffolds. N=9 control samples, n=3 samples at 10 minutes, and n=6 samples at 1 hour. Data are average±SD with two-way ANOVA and Fisher's LSD multiple comparisons test, p≤0.05 considered significant;

FIG. 6e shows a mosaic assay response to Adriamycin dose test;

FIG. 6f illustrates quantification of Adriamycin dose test on NT (light grey) and HF (dark grey) scaffolds. N=3 samples

FIG. 6g shows a mosaic assay response to patient pre-transplant serum after incubation for 3, 24, and 48 hours, for patients with recurring and non-recurring post-transplant focal segmental glomerulosclerosis (FSGS);

FIG. 6h illustrates quantification of patient serum-exposed mosaic assay. N=3 patients per group with one serum sample per patient;

FIG. 6i shows hot embossed polystyrene with patterns, FIG. 6j assembled to well plates for fabrication of scalable topographic plates;

FIG. 6k shows mosaic assay cells on polystyrene plastic. Data are shown as average±SD. Two-way ANOVA statistical testing, using Fisher's LSD test for multiple comparisons. p≤0.05 considered significant;

FIG. 7a shows a Koch snowflake which is a typical example of fractal shape, which can be characterized by fractal dimension (Df);

FIG. 7b shows a cast of a glomerulus from X-ray nanotomography, reproduced with permission,^{[A16]} and a single podocyte reconstructed from serial focused ion beam/scanning electron microscope (FIB/SEM) images, reproduced with permission^{[A17]};

FIG. 7c shows original images, thresholded masks, and projected traces of cross-sectional H&E histology, taken from healthy and pathological (proliferative lesions) sections. Histology reproduced with permission^{[A18]};

FIG. 7d shows original images of the different shapes (from left to right): tree, podocyte^{[A13]}, Koch snowflake, glomerular trace, circle, line;

FIG. 7e shows binary images of different shapes with the background removed (unit: pixel). Original thickness of a circle and a line is 2 pixels;

FIG. 7f shows the fractal dimension range graphed over the box size, calculated by the box-counting method;

FIG. 7g shows the FFT of the various shapes of FIG. 7f;

FIG. 7h shows surface plotting of the FFT results of FIG. 7g;

FIG. 7i shows profile plots through the center of the FFT surfaces of FIG. 7g;

FIG. 7j is an illustration of the generation of topographical substrates with glomerulus-mimicking pattern from design to fabrication according to the second example study;

FIG. 8a are scanning electron micrographs (SEM) and 3D display of digital micrographs of PDMS scaffolds showing successful transfer of non-topographic (NT), round non-fractal (RT), and biomimetic low and high fractal (LF and HF) patterns to substrates;

FIG. 8b show immunostaining of podocin (counterstained with WGA) from mouse podocytes grown on NT, RT, LF and HF substrates and differentiated for 3 and 14 days;

FIG. 8c show quantification of immunofluorescence signal, where n=3 scaffold samples;

FIG. 8d shows relative gene expression of Nphs1 to Gapdh in mouse podocytes cultivated on n=5 ST and n=4 HF scaffolds;

FIG. 9a shows a SEM of podocytes cultivated on NT, RT, LF and HF scaffolds from low to high magnifications. Arrows point to finger-like apical protrusions;

FIG. 9b illustrates Podocin presence on substrates in z-stacks (n=3) where wheat germ agglutinin (WGA) stains cell membrane;

FIG. 9c shows Western blot of podocalyxin from podocytes cultivated on NT, RT, LF and HF scaffolds;

FIG. 9d shows quantification of PODXL signal from western blot (normalized to GAPDH) with n=3 scaffold samples, where one-way ANOVA statistical testing shows no significant difference;

FIG. 9e shows TEM of EVs isolated from culture media of podocytes cultivated on NT and HF scaffolds

FIG. 9f shows Western blot of ALIX and CD63 from EV samples isolated from culture media of mouse podocytes cultivated on NT and HF scaffolds;

FIGS. 9g and 9h show quantification of ALIX and CD63, respectively, signals from western blot (normalized to total protein), where n=3 samples, day 14. Student t-test was used for statistical testing and data are shown as average f SD with p≤0.05 considered significant in all tests;

FIG. 10a shows SEM of high density fractal PDMS substrates in concave and convex directions, where low and high magnifications are shown;

FIGS. 10b and 10c show Mosaic assay of non-conditionally immortalized human podocytes cultivated on concave and convex HF scaffolds (n=3) for a period of 5 days and imaged by epifluorescence (seeding densities at 100,000 and 200,000 cells/cm²) (10b) and confocal microscopy (seeding density at 100,000 cells/cm²) (10c);

FIGS. 10d and 10e show quantification of cell numbers and whole cell morphology, respectively, by fractal dimension of Mosaic assay human podocytes cultivated on concave and convex HF scaffolds (n=3) for 5 days;

FIG. 11a shows hot embossed tissue culture polystyrene substrates with high density HF patterns;

FIG. 11b shows high density HF patterns covering the entire bottom in a 24-well format;

FIG. 11c shows Mosaic assay of non-conditionally immortalized human podocytes after 4 days of cultivation on patterned polystyrene substrates at 100,000 or 200,000 cells per well with and without Matrigel coating, scale bar: 200 μm;

FIG. 12a shows unsupervised principal component analysis (PCA) delineating clusters in total gene expression patterns for four main conditions: baseline, NT, RT, and HF;

FIG. 12b shows pluripotency scores compared to published datasets of undifferentiated induced pluripotent stem cells (iPSCs), iPSC-derived progeny, adult podocytes and an immortalized cell line^{[A6]};

FIG. 12c shows PluriTest which ran separately on baseline, NT, RT and HF samples;

FIGS. 12d and 12e show KeyGenes analysis comparing baseline cells (frozen) and different culture conditions (NT, RT and NF) to the (12d) adult stage of development and (12e) kidney identity^{[A6]};

FIG. 12f is a Venn diagram of differentially expressed genes in the three culture conditions (NT, RT and HF) compared to the baseline cells, prior to cultivation

FIGS. 12g-i are Venn diagrams illustrating the number of upregulated overlapping and distinct gene ontologies (GO) terms for (12g) biological processes, (12h) cellular components and (12i) molecular function

FIGS. 12j and 12k show the top 10 most upregulated GO terms for biological processes, cellular components and molecular function in (12j) HF vs NT and (12k) RT vs NT. *GO terms exclusive to HF vs. NT and not significant in RT vs. NT;

FIGS. 12l-p are Venn diagrams illustrating the number of downregulated overlapping and distinct gene ontologies (GO) terms for (12l) biological processes, (12m) cellular components, and (12n) molecular function.

FIGS. 12o and 12p show the top 10 most downregulated GO terms for biological processes, cellular components and molecular function in (12o) HF vs NT and (12p) RT vs NT;

FIG. 12q shows a supervised hierarchical heatmap of ECM, matrix remodelling, cell adhesion and signaling genes relevant for podocyte function with significant differential expression;

FIG. 13a show YAP interactome. The nodes represent the related genes identified by GeneMANIA, with the size of each node indicating the rank of how closely the gene was associated with YAP1. The node shading are used to relate RNA seq data to the YAP1 interactome;

FIG. 13b is a heatmap of significantly different genes from YAP interactome;

FIG. 13c shows Mosaic assay on HF scaffolds treated by YAP inhibitor verteporfin;

FIG. 13d shows Lactate dehydrogenase (LDH) release of Mosaic assay cells inhibited with verteporfin at control, 1 μM, and 2 μM. n=8 samples per group; and

FIG. 13e shows quantification of whole cell morphology by fractal dimension of Mosaic assay cells on HF scaffolds inhibited with verteporfin. n=3 samples per group.

Similar reference numerals may have been used in different figures to denote similar components.

### DESCRIPTION OF EXAMPLE EMBODIMENTS

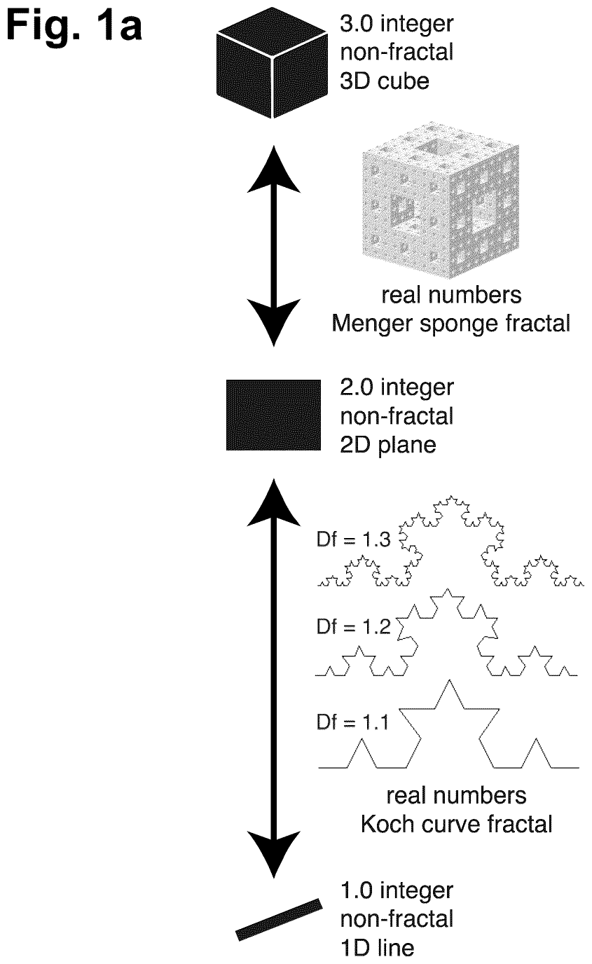

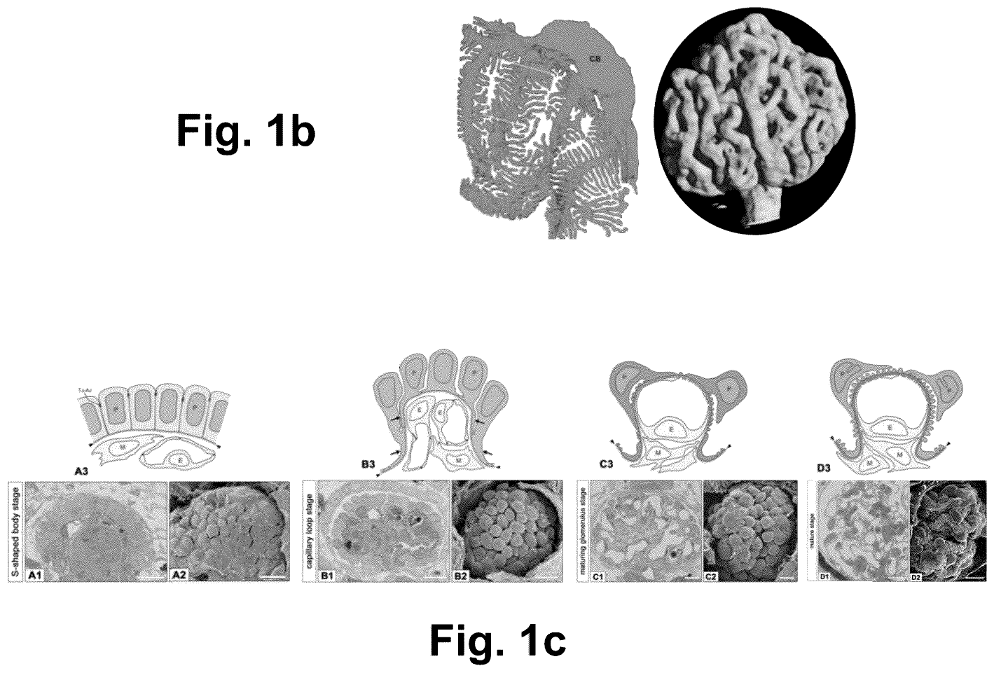

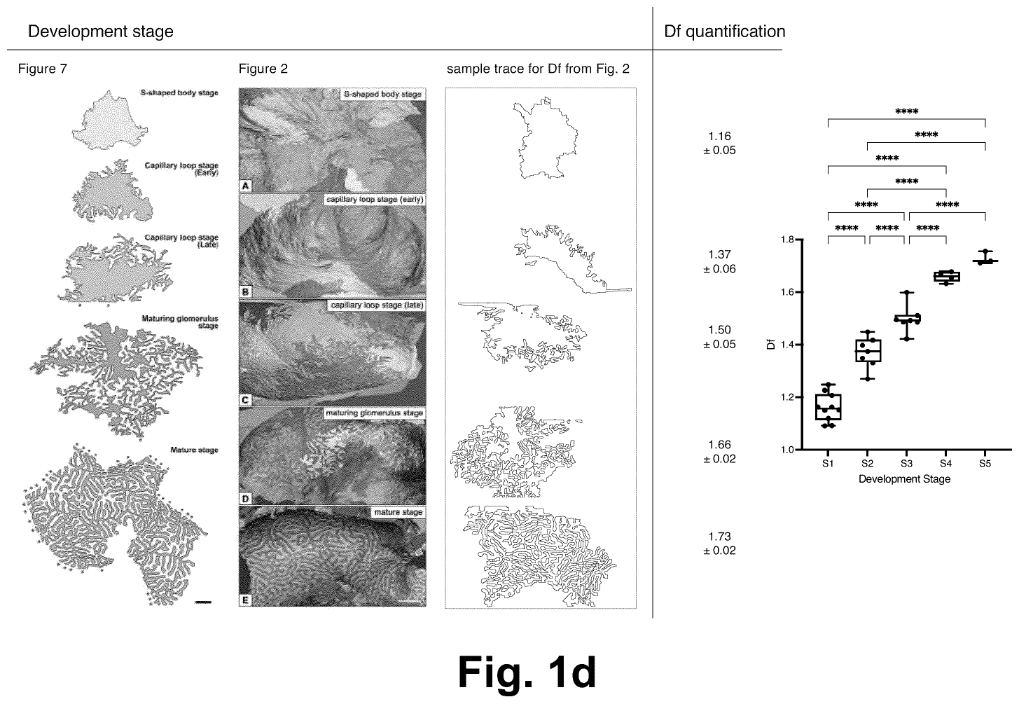

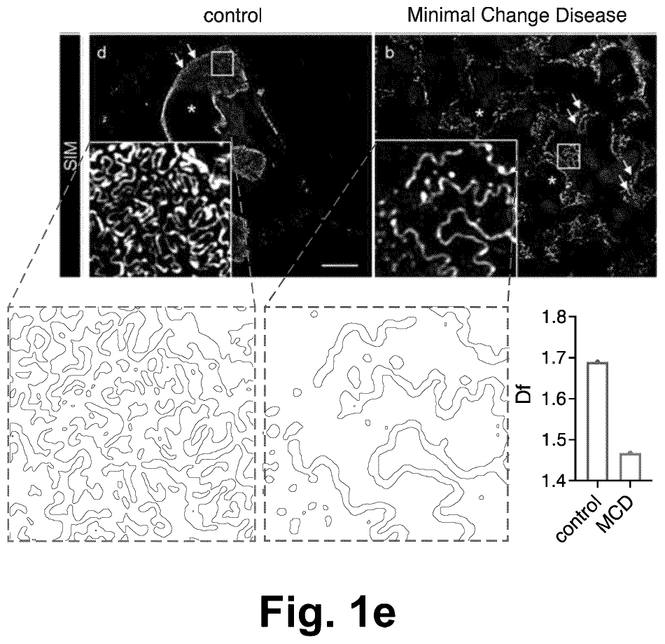

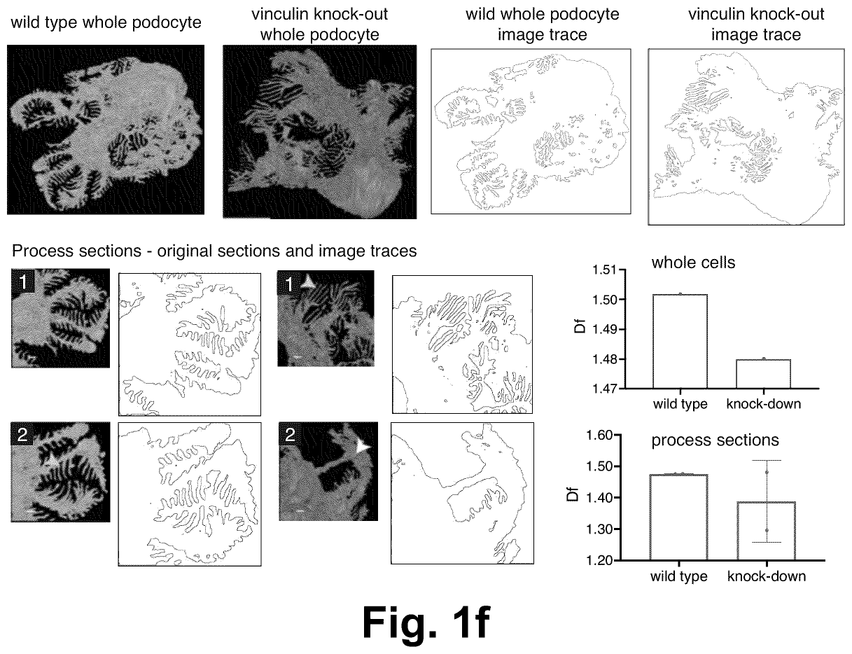

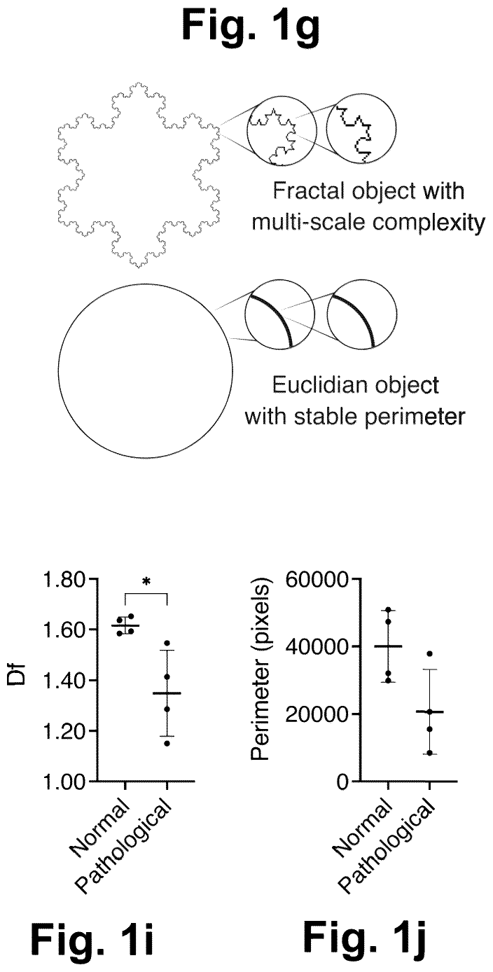

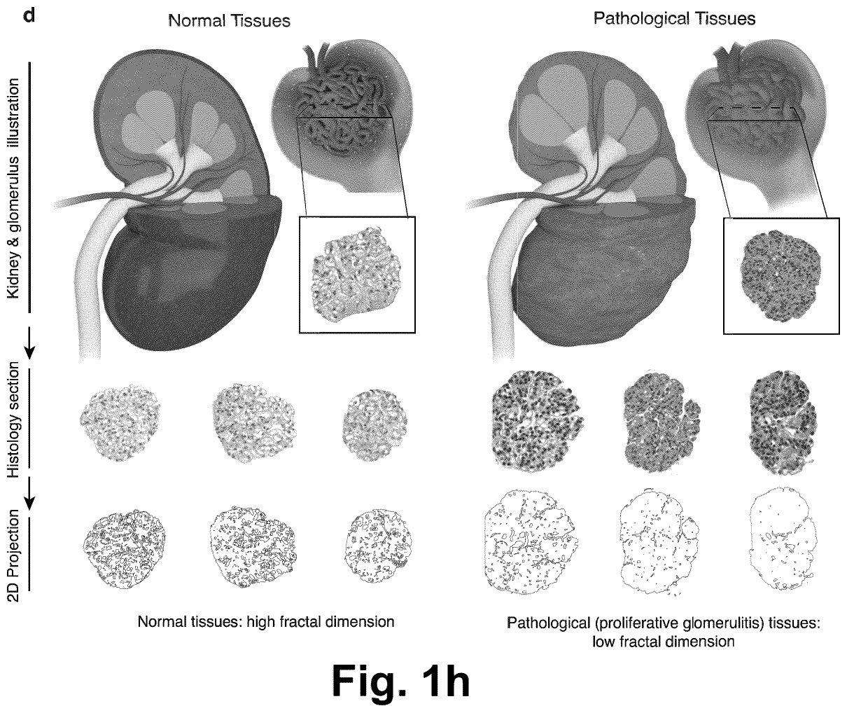

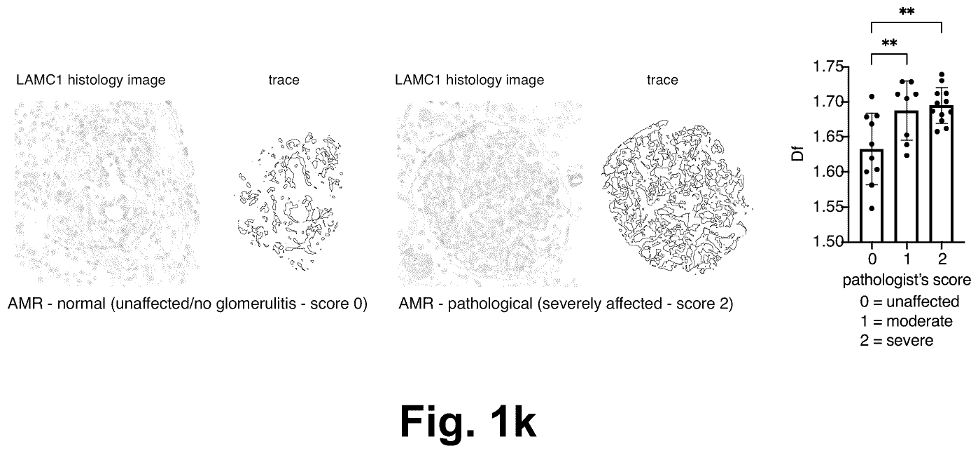

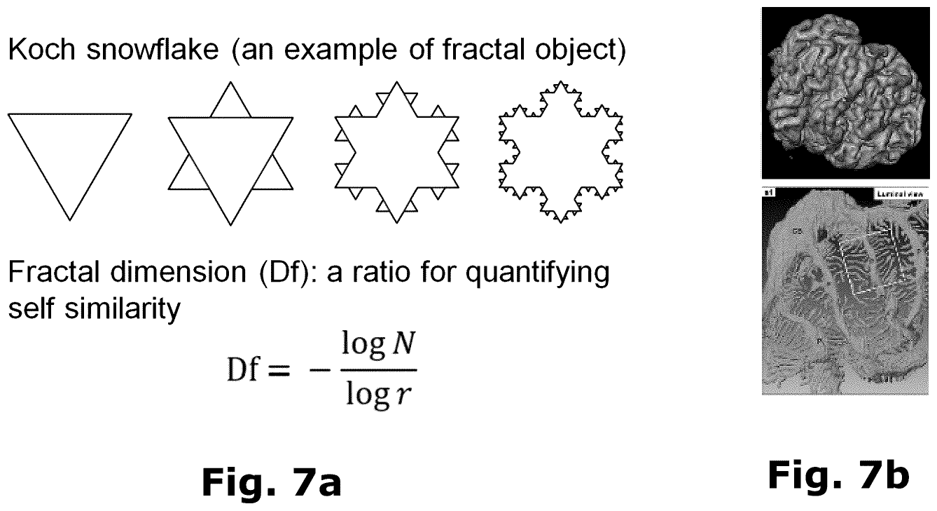

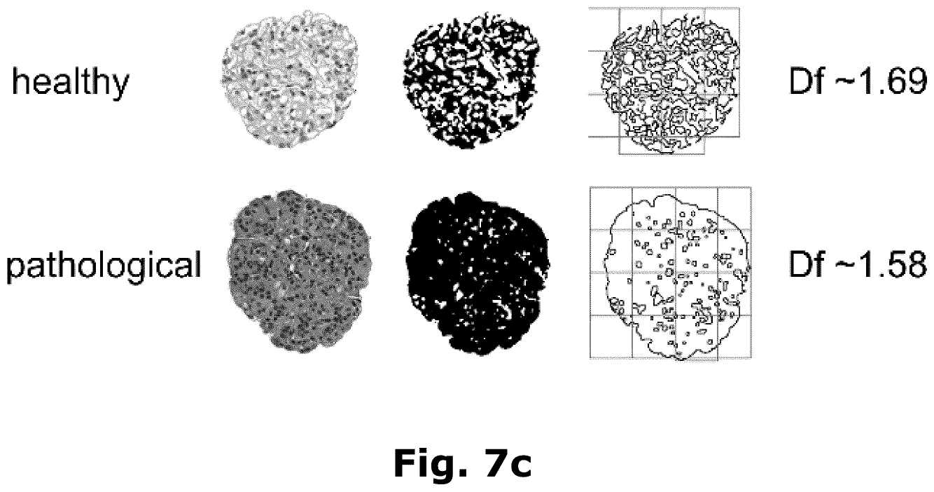

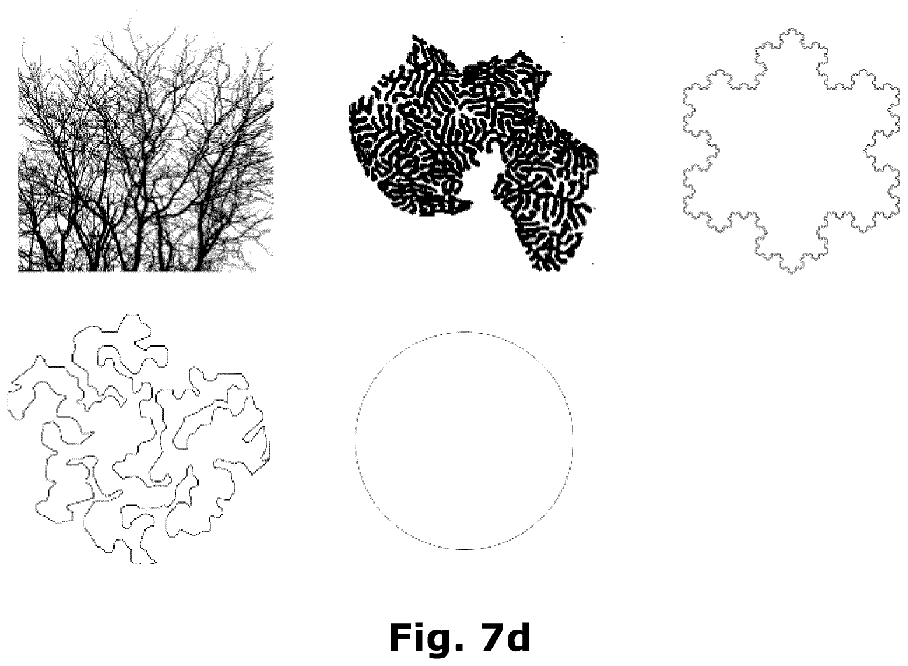

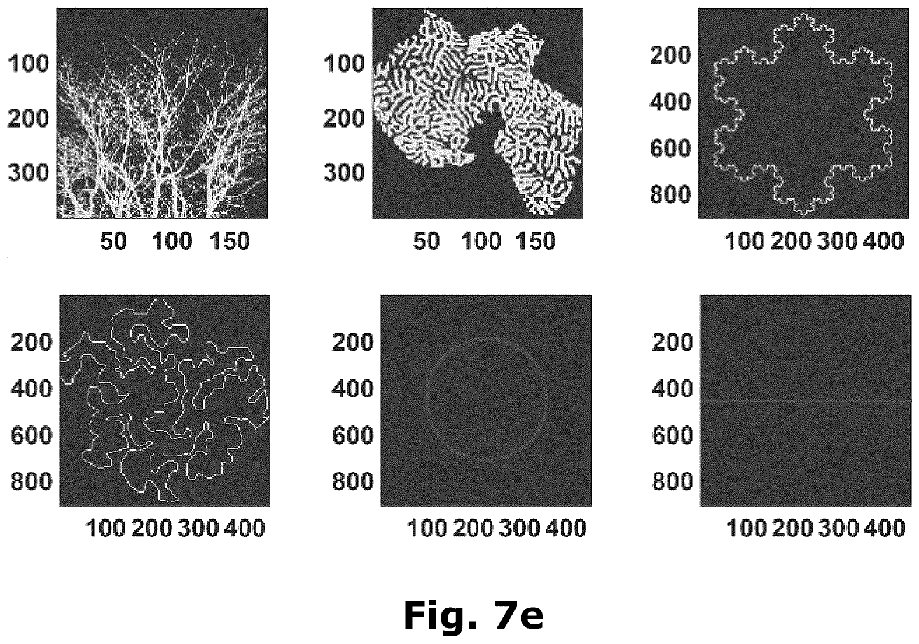

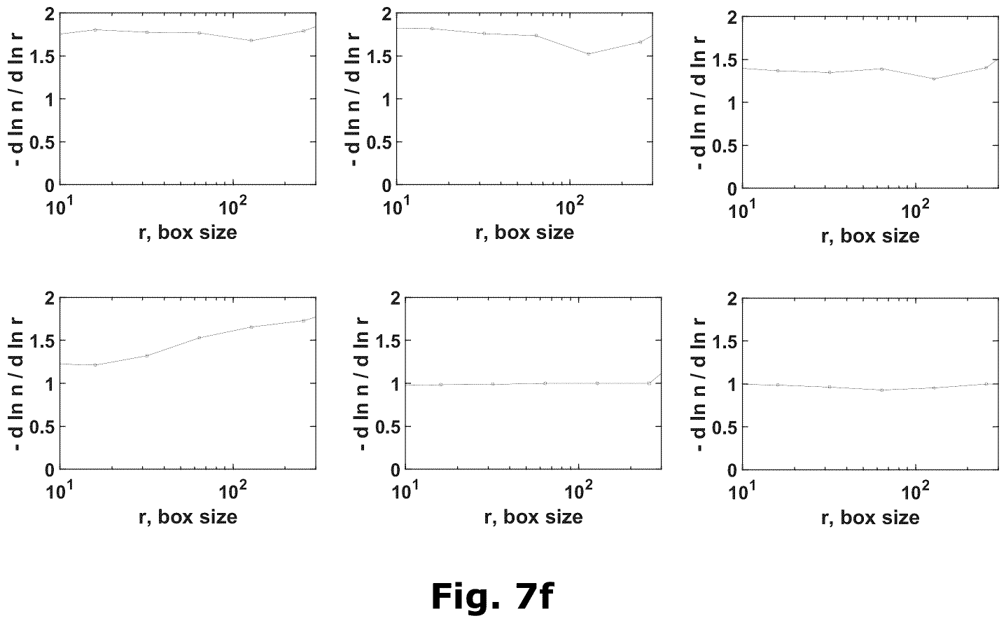

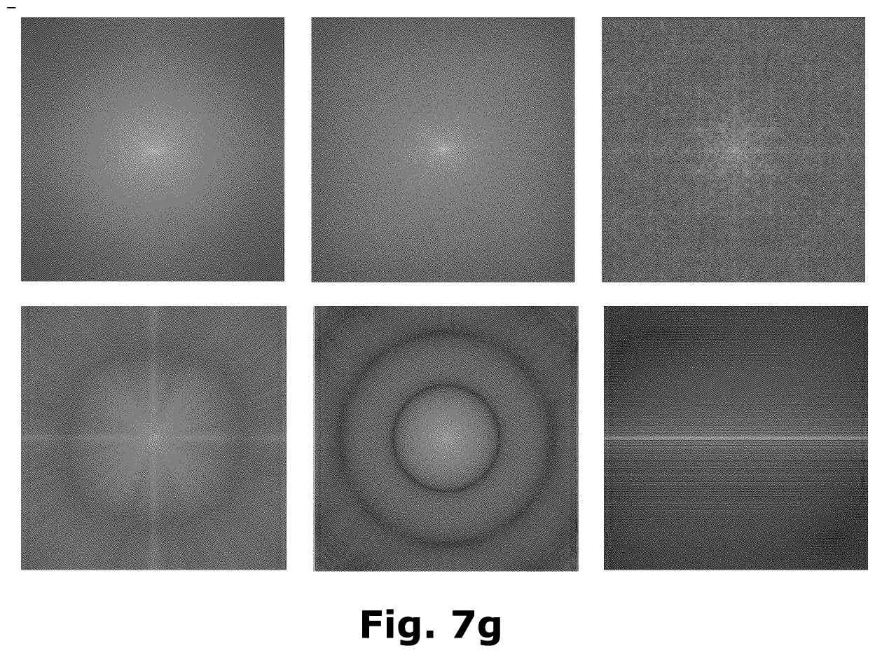

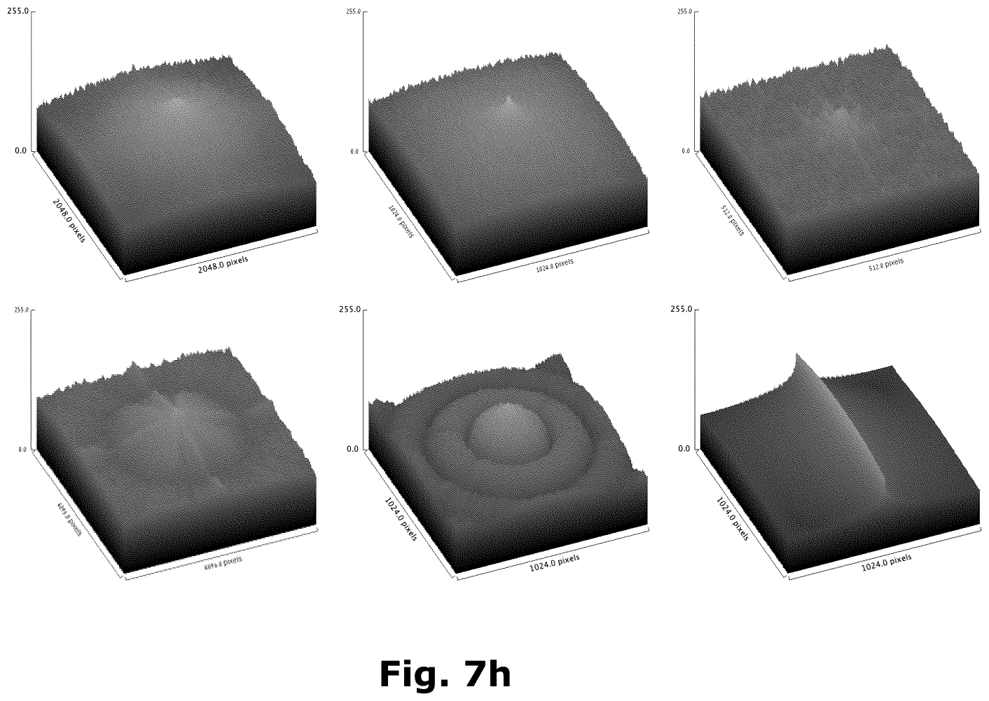

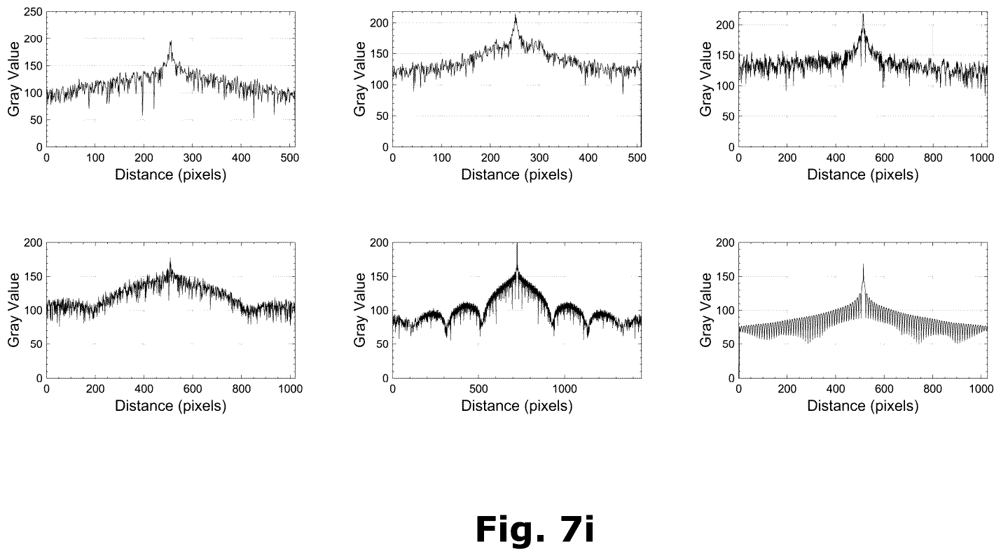

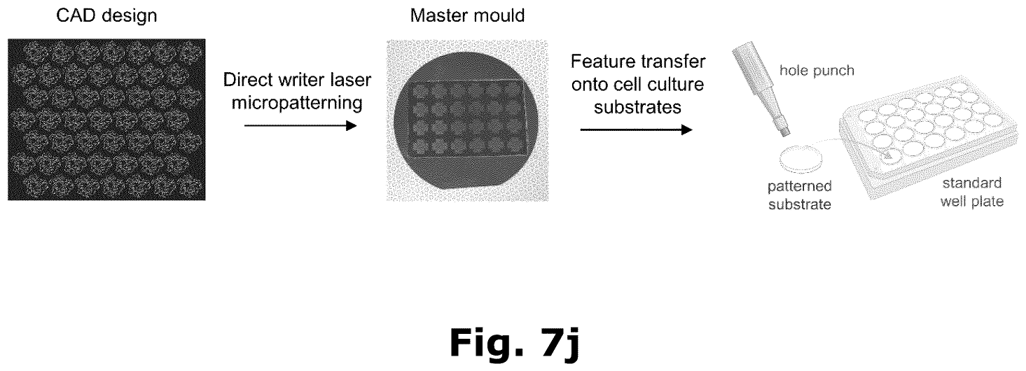

A powerful manifestation of size control on multiple scales in nature is fractal patterning, where subparts of fractal structures resemble the whole¹. These ubiquitous “fractional” dimensional forms (FIG. 1a) are believed to result from optimization of environmental factors, such as the need for efficient mass transport². Indeed, the state of tissue health has documented correlations with fractality of its form, suggesting that there is a “healthy dose of chaos” in biological systems³. Most often, fractality is simply used to characterize the geometry of complex forms. A typical fractal shape shows self-similarity over multiple scales of measurement, such as the famous Koch snowflake, whose size expands 4 times as the scale decreases by ⅓, corresponding to a fractal dimension of 1.26 according to the formula (FIG. 7a).

Fractals exhibit power-law relationships between size and measurement precision: as a fractal's structure is measured more precisely, the output increases, whereas non-fractal Euclidean objects have outputs that converge to a steady value (FIG. 1g). Thus, fractal dimension (Df), a quantification of the scaling relationship in a given form, is better suited for characterizing complex structures like fractals that escape linear measurement approaches (FIG. 1g)¹⁵.

Both the glomerulus and podocyte demonstrate a complex structure under X-ray nanotomography and focused ion beam/scanning electron microscopy (SEM), respectively, suggesting fractal dimension as a suitable index for the quantification of their geometry (FIG. 7b). Preliminary fractal analysis of histology images of glomeruli from healthy and pathological samples also showed the existence of fractality and a change in fractal dimension between healthy and diseased states (FIG. 7c).

To confirm the occurrence of fractality, the planar projection of a SEM image of a podocyte^{[A13]} and an outline of a glomerulus obtained from histology^{[A14],[A15]} were compared to objects that are widely accepted to be fractal, specifically a branching tree and Koch snowflake, a common fractal computational control. These were contrasted with the objects that are known not to be fractal, specifically the perfect circle and the straight line (FIG. 7d). FIGS. d-i show characterizations of fractal properties in a podocyte^{[A13]} and a glomerulus in comparison to standard fractal (a tree, a Koch snowflake) and non-fractal (a line and a circle) controls. Relying on the box counting method, it was determined that both the mature podocyte and the outline of a glomerulus exhibit multi-fractality, similar to that of a branching tree and of the Koch snowflake (FIGS. 7f, 7g). Whereas the non-fractal objects, a circle and a line, exhibited a Df=1 regardless of the box size, the podocyte (Df ranging from 1.52 to 1.82) and the glomerulus (Df ranging from 1.21 to 1.73) exhibited a Df similar to values observed for the tree (Df from 1.68 to 1.80) and the Koch snowflake (Df from 1.27 to 1.39).

Importantly, whereas dominant frequencies were easily identifiable for the circle and the line (FIGS. 7g-i) upon fast Fourier transformation (FFT), no dominant frequency was apparent for the podocyte, the glomerulus, a tree and the Koch snowflake (FIGS. 7g-i). Together with the fractal analysis (FIGS. 7c-i), these results further confirm the presence of fractality both in the adult, mature, podocyte shape, as well as histological outline of the glomerulus.

The present disclosure relates to using fractal patterning of topographical cues to support optimal tissue form and function, using podocytes and the kidney glomerulus as a model system. Using kidney podocytes as a model system, bioinspired templating of glomerular histology has been leveraged to design controlled fractal 2½-D surfaces for cell culture.

As noted above, podocytes are terminally differentiated epithelial cells that wrap around glomerular capillaries and exhibit fractally-bifurcating processes responsible for sieve-like barrier function during blood purification. Podocytes increase their structural complexity as capillaries loop from flat to bundled geometries during development⁴, suggesting fractality in the podocyte-glomerulus system (FIGS. 1b to 1f).

The methods and apparatus disclosed herein may enable more robust and consistent formation of morphological and/or functional features (e.g., slit diaphragms, foot process formation and interdigitation, among others) in podocyte cultures in vitro and measurements of permeability. The disclosed methods and apparatus may also be suitable to promote more physiological development of other cell types in vitro. Examples disclosed herein may enable greater control of cell microenvironment via topographical and biochemical cues that enable podocytes to achieve a physiological phenotype in vitro, as measured by the presence of slit diaphragm proteins, including nephrin, and the adoption of a morphological profile more redolent of in vivo podocytes.

Examples disclosed herein may be applicable for study of the kidney, and may help to improve kidney research by providing a biomimetic culture system that will enable podocytes to mature to more physiological extents. Culture of other cell types may also benefit from the present disclosure, which may help to improve study of other organs including, for example, lungs, blood vessels, bone marrow, or any other tissue having a fractal topology.

In the present disclosure, fractal dimension analysis is applied to evaluate the structure of the native glomerular environment. Hematoxylin and eosin (H&E)-stained glomerulus histology images obtained from the literature¹⁶ were processed to outline, as a first approximation, the capillary patterns of healthy and proliferative glomerulitis sections (FIG. 1h). By fractal dimension box-counting quantification, a distinct variation in fractal complexity of outlined glomerular capillaries was discovered, where healthy traces had higher Df than pathological (FIG. 1i). By contrast, a Euclidean approach to measuring the perimeter of the contrasted images was less effective at detecting subtle changes in structural complexity (FIG. 1j). Laminin-stained sections from biopsies of patients with antibody-mediated rejection (AMR), a complex injury that occurs to transplanted kidney grafts characterized by microvascular inflammation, endothelial complement deposition, and a circulating anti-body against the donor graft¹⁷, also demonstrated a correlation of Df with pathologist's grading of glomerulitis severity (FIG. 1k). Fractal dimension quantification of outlined glomeruli correlates with pathologist scoring of glomeruli, from unaffected to severe glomerulitis. Depending on type of injury and the feature being assessed, fractality of pathological tissues may be higher or lower than normal tissues. N=5 patients biopsies and 3-8 glomeruli assessed per patient^{S4}. Data shown as average±SD. Analysis by one-way ANOVA and Fisher's LSD multiple comparisons test, p≤0.05 considered significant. AMR=antibody mediated rejection. These results support that Df was sensitive in capturing disease effects on glomerular tissues, and presents a new quantitative and unbiased approach to characterizing glomerular injury.

Complex tissues operate in three-dimensional space, yet standard cell culture methods are faced with dimension reduction, e.g., well plates designed for monolayer culture and analysis. A system was sought that provides biomimetic shape cues in space that could be specifically tuned to test the role of fractal patterning, while remaining compatible with standard cell culture handling protocols.

The present disclosure presents example biomimetic techniques that may help to overcome or raise this plateau of the culture system to achieve more useful, higher-fidelity levels that better match in vivo maturity. Podocytes grown on glomerulus-mimicking fractal topography were found to express higher levels of functional markers and exhibit enhanced cell polarity.

### Cell Culture Apparatus

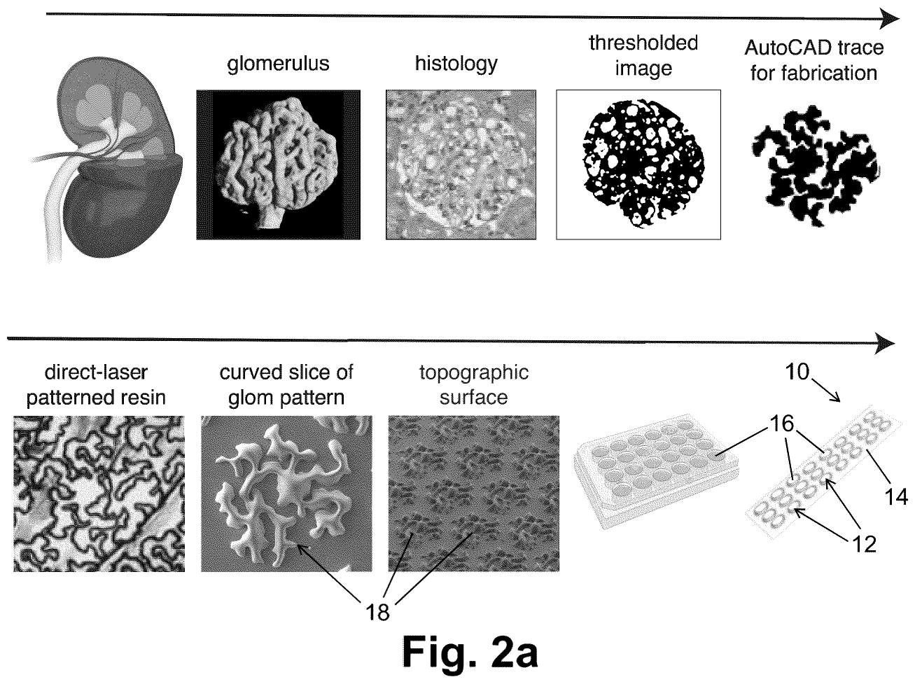

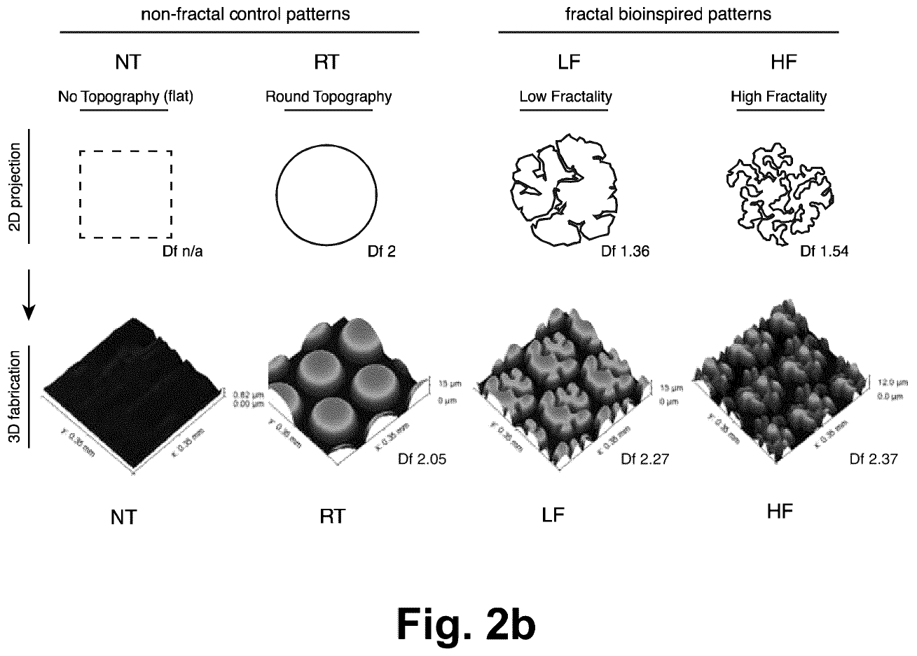

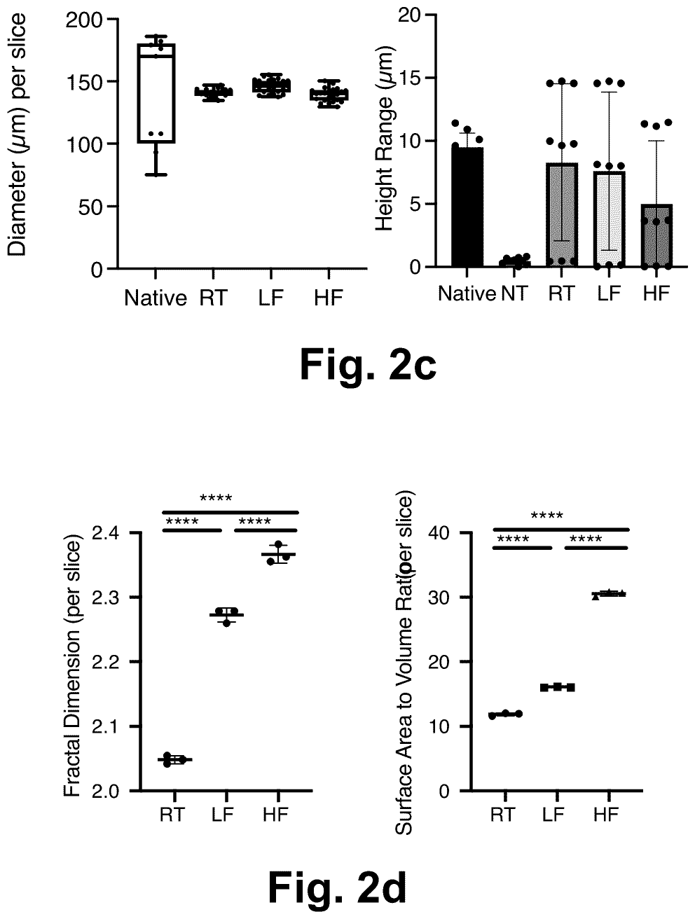

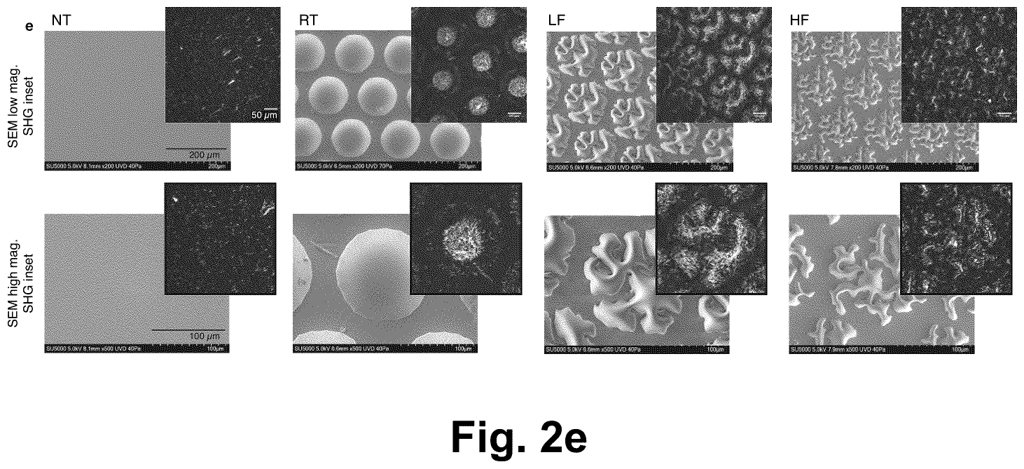

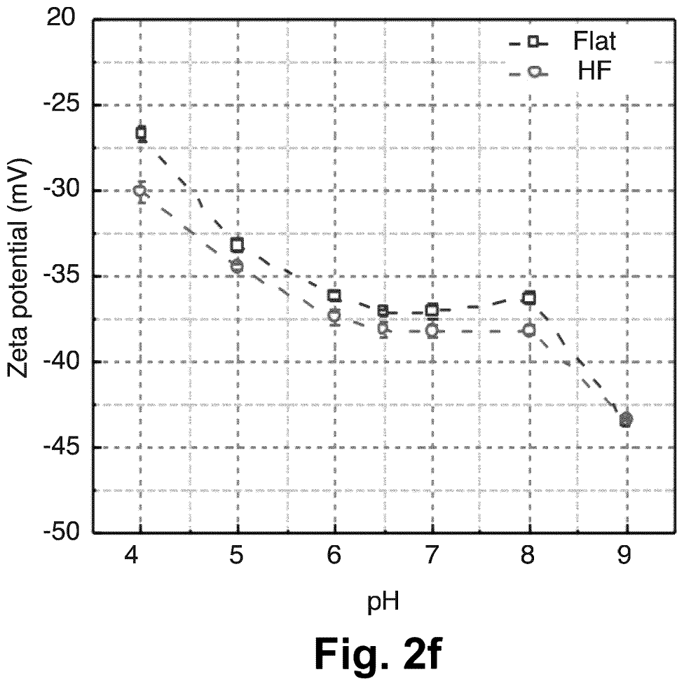

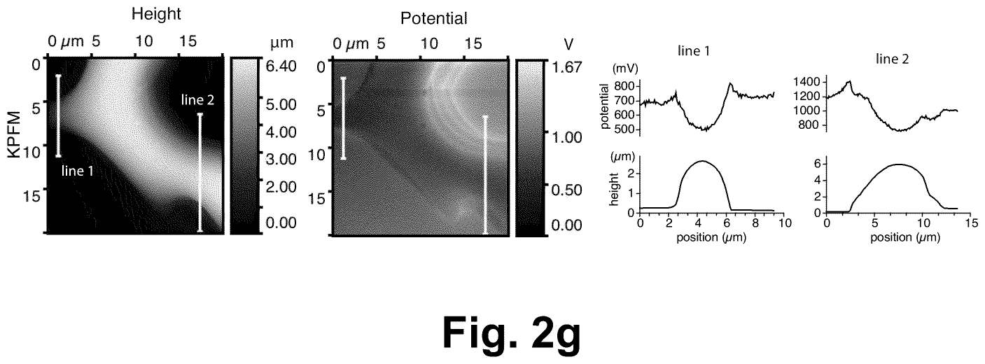

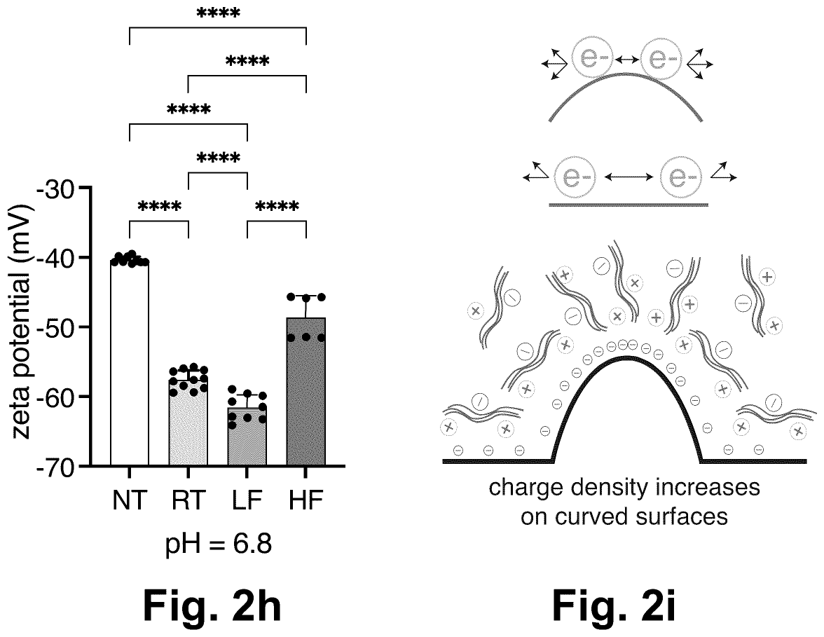

The present disclosure describes a cell culture apparatus 10 that may be formed using microfabrication techniques described below. The cell culture apparatus 10 generally includes at least one topographic scaffold 12, which may be bonded to a glass microscope slide 14. In the embodiment depicted in FIG. 2a, the cell culture apparatus 10 comprises twenty-four topographic scaffolds 12.

The cell culture apparatus 10 disclosed herein includes a first chamber, for cultivating cells, which supports the one or more topographic scaffolds 12, each which supports a surface 16 for cell cultivation thereon. The surface 16 exhibits one or more fractal features 18, where each fractal feature 18 comprising out-of-plane fractal patterning that provides non-planar microtopology for the surface 16. Each fractal feature 18 may includes protruding Gaussian, fractal micro-curvatures, such as that shown in FIG. 2a.

Complexity of each fractal feature may be determined by a fractal dimension (Df) measurement, which may be calculated using the equation:

$$Df = \frac{- log N}{log ε}$$

wherein N is the number of branching units, and ε is the scaling factor (e.g., based on the magnification used to view the fractal feature). Conceptually, this means that if the number of branching units increases as the view is zoomed in, the fractal dimension Df should also increase.

The fractal patterning may be derived from a histological section of a biological microenvironment exhibiting the fractal patterning. In the example of FIG. 2a, the biological microenvironment is a podocyte microenvironment, where the fractal patterning mimics glomeruli in the podocyte microenvironment and the micro-curvatures are patterned after its convoluted capillary shapes.

In such applications, the Df of each fractal feature 14 may be at least 2.2, and is preferably at least 2.3. A higher fractal dimension indicates a higher level of complexity in the fractal patterning. It may be noted that the level of fractality (e.g., as measured by Df) that would be desirable may be dependent on the biological microenvironment. In the case of podocytes, an average diameter of the fractal patterning of the one or more fractal features 14 may be between 140 to 160 μm, and may preferably be 150 μm. Peaks in the fractal patterning of each of the fractal features 14 may also be between 5 to 15 μm.

While the biological microenvironment described is a podocyte microenvironment, other biological microenvironments with fractal patterning may be used as a template to generate the cell culture apparatus 10. Other such biological microenvironments includes bronchial epithelial, blood vessel, lung, or bone marrow microenvironments.

### First Example Study

Method of Fabricating Cell Culture Apparatus

In some examples, a microfabrication technique is provided to create the fractal patterned microscope slide/apparatus 10 for cultivation of more useful, higher-fidelity level cells (see FIG. 2a, for example).

First, a fractal pattern is templated onto a substrate. A histological section of a biological microenvironment with fractal patterning may be provided, and templated onto the substrate using photolithography. For example, the histological section may be thresholded, traced for fabrication using AutoCAD, and direct-laser patterned onto resin. In the example illustrated in FIG. 2a, the biological microenvironment is a podocyte microenvironment, where an example glomerulus exhibits the fractal patterning. The convoluted capillary shapes were patterned across a 2D substrate using maskless photolithography. While not illustrated in the figures, the biological microenvironment (that provides the histological section to be patterned onto a substrate) may instead be any biological microenvironment that exhibits fractal patterning, such as a bronchial epithelial, a blood vessel, a lung, or a bone marrow microenvironment.

After the fractal pattern is templated onto the substrate, non-planar microtopology is generated on the substrate according to the fractal patterning. For example, protruding Gaussian and fractal micro-curvatures may be generated according to the fractal patterning. In this manner, the fractal curvature is controlled and precisely tunable.

An inverse mold is then formed by curing a first polymer over the non-planar microtopology with fractal patterning. A surface (along with a base) for cell cultivation is then formed by curing a second polymer using the inverse mold, the surface being formed to exhibit the one or more fractal features, each fractal feature comprising the out-of-plane fractal patterning providing non-planar microtopology for the surface. The second polymer may be polydimethylsiloxane (PDMS), polystyrene, or poly(octamethylene maleate (anhydride) 1,2,4-butanetricarboxylate) (124-polymer). In the some applications, the second polymer may be 124-polymer with an inert polymer incorporated therein. In such cases, the method may further comprise leaching out the inert polymer after curing. The resulting surface with the fractal patterning may effectively be considered 2½-D “slices” of glomeruli (as shown in FIG. 2a) arrayed over the culture surface.

The surface and the base collectively constitute a topographic scaffold. The topographic scaffold may then be secured/bonded to a glass microscope slide, thus forming a cell culture apparatus that presents nonlinear 3D structure at the scale relevant for cells, while allowing for standard 2D handling techniques.

Example Fabrication

Glomerular histology tracing: Images of kidney histology sections from the literature and from biopsied patient samples were collected. Images were manually cropped to only show glomeruli. In Adobe Photoshop, the magic wand tool was used at a tolerance of 30, with anti-alias activated and contiguous deactivated. The background white space corresponding to Bowman's capsule was selected as the reference colour to outline the cellular/protein spaces. A mask was created using the following specifications: edge radius 0, smooth 0, feather 1.0, contrast 100, and shift edge 0%. The binary mask was exported and then outlined in ImageJ using a line thickness of 1 pixel. Outlined traces were quantified in ImageJ using the FracLac plugin. Box Counting, on binary images with the background locked to white, was applied for fractal analysis.

Scaffold design and fabrication: Select histology images were additionally processed for scaffold fabrication to satisfy fabrication constraints, including a minimum feature resolution of 1 μm and a need for interconnected lines. In Photoshop, histology images were posterized with dark and midtone levels made equal, thresholded, and then eroded and dilated until gaps and noisy artifacts were reduced. Images were then placed in Illustrator and traced with anchor points set to below 800 and curves were snapped to lines and paths simplified to straight lines. These traces were next imported to AutoCAD. Lines were converted to polylines and joined. The glomeruli traces were then arranged manually in a hexagonal packing to make packing as close as possible, then arrayed to a 16×16 mm square. The squares were then directly laser-patterned onto AZ P4620 positive photoresist using a MicroWriter ML3 Baby. Silicon wafers were first coated with an HMDS adhesion layer by spinning at 3000 rpm for 30 seconds, softbaked at 150° C. for 1 minute. AZ P4620 was then spin coated at 1500 rpm for 30 seconds, softbaked at 110° C. for 80 seconds, then rehydrated for approx. 30 minutes at room temperature. Designs were patterned using 770 mJ/cm2 with 0 μm focus correction, then developed with the help of a sonicator. Patterns were then re-flowed to generate curvature by baking at 120° C. on a hot plate for 60 seconds.

PDMS molding: Patterns from AZ4620 master molds were inverse molded with PDMS at a 1:10 ratio, cut to squares, then plasma bonded to silicon wafers with the edges sealed using polyurethane glue. These PDMS inverse molds were then fully cured at 120° C. and used as a master mold to make topographic PDMS replicates, used as scaffolds for cell culture. Scaffolds were punched out from the PDMS to create circular inserts, autoclaved, placed in the well-plates, rinsed with PBS, then coated with matrix proteins for 1-2 hours in the 37° C. incubator prior to cell culture.

Profilometry: 3D scans of the scaffolds were conducted using a KLA Tencor AlphaStep Profilometer D-120. PDMS scaffolds were measured in 3D scan mode using matrix setup, with a 2 μm step size in the x- and z-directions, 400 μm scan length in x- and z-directions, a 100 μm dynamic range, 0.1 mm/sec speed, step up and down, stylus force of 0.03 mg, and data filter level set to 16 data points. The scan data was loaded to and processed using Gwyddion. Data was levelled using intersections with given lines applied to the valley regions between patterns, horizontal scars removed, and data minimum value shifted to 0. For a single encircled pattern or “slice,” the surface area, volume, and average/minimum/maximum/peak/valley heights were extracted for scaffold characterization. Volumetric fractal analysis was applied using the software's partitioning method.

Cultivating and Imaging the Cells

The present disclosure further describes a method for cultivating cells using the cell culture apparatus 10 as described above. It was found that use of the cell culture apparatus 10 to cultivate cells helps to promote differentiation of the cells in culture.

Cultivation

A suite of four biomimetic scaffolds was fabricated and molded (as described above) in polydimethylsiloxane (PDMS) (FIG. 2b): (NT) was a flat, “No Topography” control scaffold; (RT) was a non-fractal “Round Topography” control featuring a simple array of microhemispheres; (LF) was a fractal scaffold exhibiting “Low Fractal” complexity, traced from a pathological glomerulus; and (HF) was a “High Fractal” scaffold traced from a healthy glomerulus. The order from NT, RT, LF, to HF may be conceptualized as a dose-test of complexity, from non-structured to increasingly complex fractal structures similar to the developmental timeline (FIG. 1c). Pattern size was controlled by average diameter (approx. 150 μm), which matched native values, and by range of peak feature heights (5-15 μm) per pattern (FIG. 2c)^{18,19}. Fractal dimension (Df) was varied, as corroborated by surface area to volume ratio, thus supporting increasing complexity presented per unit of space (FIG. 2d). These scaffolds 12 with “micro-architected fractal topography” may be termed “miCRAFT”.

Cell culture: Three podocyte cell sources were used in this work: murine podocyte E11 cell line (referred to as mouse pocoytes); human Podo/tert 256 cell line (referred to as human podocytes); and primary human fetal podocytes from GW 18 (referred to as primary human podocytes). In general, cells were expanded and subcultured in collagen I-coated T175 flasks until 70-85% confluence, before passaging with pre-warmed 0.05% trypsin for subculture. Cells from passages 3-12 were used in the study, and biological replicates were thawed from separate vials at different passage numbers. The conditionally immortalized E11 murine podocyte cell line was a kind gift from GSK. Mouse cells were seeded at an initial density of 50,000 cells/cm² and allowed to attach and reach 80% confluence under proliferative conditions at 33° C. with 10 units/mL interferon gamma supplementation in RPMI 1640 culture media containing HEPES and GlutaMAX, with 10% FBS, and 1% pen-strep. Differentiation was induced by switching cells to 38° C., removing interferon gamma, and switching culture media to DMEM-F12 with 10% FBS and 1% pen-strep for an additional 14 days prior to end point analyses. The Podo Tert256 human podocyte hTERT immortalized cell line (Evercyte) was grown at 37° C. using MCDB101 basal medium, supplemented with 20% FBS, 2 mM GlutaMAX, 12 μg/mL Bovine Brain Extract, 10 ng/mL hEGF, 25 ng/mL hydrocortisone, and 100 μg/mL G418. Human cells were seeded at an initial density of 40,000 cells/cm², and grown until 80% confluence as the study endpoint (4-5 days). Primary human podocytes GW 18 (Lonza, on-demand limited release) were used for RNA sequencing and seeded at an initial density of 30,000 cells/cm² at passage 7, using RPMI 1640 basal medium with GlutaMAX and HEPES, supplemented with 10% FBS, 1% ITS, and 0.5% GA-1000. Primary cells were rinsed with HEPES-BSS during subculture and rinsing steps. Primary cells were grown until confluence for the study endpoint (3 days).

Coatings: Coatings were applied to scaffolds in solution using 0.4 mL per well of a 24-well plate. Matrigel (Corning, #354234) was diluted 1/60 in basal media and incubated for 2 hours at 37° C. Rat tail collagen I (Corning, #354236) was diluted in PBS at 0.1 mg/ml for 1 hour at 37° C. For SHG, coating was 2 hours at room temperature. Human collagen I (Sigma, #C7774) was diluted to in PBS at 6 μg/cm2 and incubated for 1 hour at 37° C. Porcine kidney ECM (Xylyx Bio, NativeCoat Kidney ECM surface coating kit #MTSKY201) was diluted in water and kit buffers according to manufacturer's instructions, at 0.08 mg/mL, and incubated for 1.5 hours at 37° C. Laminin 511 (Nippi Matrixome, Easy iMatrix-511 #892018) was diluted in PBS at 0.25 μg/cm2 and incubated for 1 hour at 37° C. For the mixed laminin/collagen coating, the two coating solutions were mixed at 50% v/v since both were diluted in PBS. Live/dead staining was conducted using CFDA-SE (Life Technologies, #C1157) and propidium iodide (Life technologies, #P3566) stains, by rinsing cells with warm PBS and incubating with 1/1000 dilution of stain in PBS for 30 min in the 37° C. incubator.

Imaging

Scanning electron microscopy and environmental scanning electron microscopy (SEM): Samples were fixed with 2% PFA for 20 minutes at room temperature. They were post-fixed with 0.5% osmium for 20 minutes in the dark at room temperature in the chemical hood. They were then serially dehydrated in ethanol, dried at the critical point (CO₂), and gold-coated for 2 minutes. Samples were imaged using a Hitachi SU-5000 scanning electron microscope, using 20 kV voltage acceleration. For environmental SEM, dry PDMS scaffolds were imaged directly using Hitachi SU5000 under environmental conditions using vacuum at 25-70 Pa and 5.0 kV voltage acceleration.

Immunofluorescent staining and signal quantification: After rinsing with warm PBS, cells were fixed with 2% PFA in PBS for 20 minutes at room temperature, and washed three times with PBS. Fixed cells were stored in the fridge or used fresh for immunostaining. Fixed cells were blocked in 5% normal goat serum (NGS) with 0.1% triton X-100 for 1 hour at room temperature on a shaker. Blocking solution was diluted to 2% NGS using PBS without triton X-100 for primary and secondary staining solutions. Between blocking and primary staining, no rinse was applied. Primary stains were applied overnight in the fridge. Between primary and secondary staining, three five-minute rinses with PBS on a shaker were applied. Secondary staining was applied for 1 hour at room temperature on a shaker. After secondary staining, samples were again rinsed thoroughly, then mounted onto microscope slides by removing from well-plate, dabbing off excess liquid from the bottom of the scaffold with paper towel, and placing on a glass slide using tweezers. Up to three samples were mounted to a single slide. Mounting media containing DAPI (Vector Laboratories, Vectashield #H-1200) was applied to each scaffold surface using 6 μL per scaffold, then a cover slip was applied directly overtop ensuring even spreading of the mounting medium.

The same antibody dilutions were applied to both mouse and human cell cultures:
1/500 dilution of Rabbit anti-nephrin (Thermo Fisher #PA520330)
1/200 dilution of Rabbit anti-NPHS2 (Abcam, #ab50339)
1/100 dilution of Rabbit anti-SYNPO (Sigma-Aldrich, #SAB3500585)
1/200 dilution of Rabbit anti-CD2AP (Fisher/Life technologies, #PA551879)
1/500 dilution of Rabbit anti-ITGB1 (Thermo Fisher Scientific, #PA5-78028)
1/500 dilution of Rabbit anti-PODX (Invitrogen, #PA5-28116)
1/500 dilution of Monoclonal mouse anti-nestin (Invitrogen, MA1-110)
1/1000 dilution of Rabbit anti-TUBA1 (Proteintech, #112241ap)
1/500 dilution of Rabbit anti-Yapi (Thermo Scientific, PA1-46189)
1/500 dilution of Rabbit anti-phospho-ILK (Sigma-Aldrich, #AB1076)

Secondary Antibodies:
1/500 dilution of Goat anti-rabbit FITC (LifeTech, #A16105)
1/500 dilution of Goat anti-rabbit Alexa Fluor 568 (Abcam, #ab175471)
1/500 dilution of Goat anti-mouse Alexa Fluor 750 (Invitrogen, #A-21037)

Counter Stains:
1/500 dilution of Rhodamine labeled Wheat Germ Agglutinin (Vector Laboratories, #RL-1022)
1/500 dilution of Alexa Fluor 488 Phalloidin (Thermo Fisher Scientific, #A12379)

Confocal Microscopy: Confocal microscopy was performed on a Zeiss LSM 880 Super Resolution Confocal microscope. Two channels were set up for DAPI/Alexa Fluor 568, and FITC/transmitted light. Z-stacks were 1 μm thick. All samples for a given experimental set were imaged in the same sitting, and the same settings were saved and reused. Criteria were used to guide sample imaging including: (1) sampled fluorescent cells had to be single, without adjacent fluorescent cells that would prevent accurate delineation, (2) sampled cells had to be within a visibly confluent area, with adjacent cells on at least half the cell body, (3) broken monolayers with rolled up cells, visible tears on the scaffold surface, or major debris were not to be sampled due to artifact. Maximum intensity projection images were used from the z-stacks for image analysis. ImageJ/FIJI was used to measure Raw Integrated Density of each signal's channel. To combine multiple sets of data, the No Topography group average was used as a normalizer. All expression signal was also normalized to cell number approximated by the number of nuclei counted in the image frame. Only images with cell density greater than 20 cells per frame were used in analysis to ensure confluence. To count cell number and approximate nuclear vs. cytoplasmic area in localization quantification, DAPI channel images were converted to binary mask images in Photoshop using the wand tool to separate background from nuclei. Nuclear regions were then selected in ImageJ using the analyze particles tool, saving the nuclear area in the ROI manager tool, then extracting nuclear signal expression of other channels using the saved regions.

Immunofluorescent Assay Method

Multi-level branching and interdigitation of foot processes, or lack thereof known as foot process effacement⁵, is one well-recognized and useful proxy for podocyte function and health. However, the ability to establish and read out confluent branching morphology in vitro is lacking due to complications of an interdigitating phenotype combined with an immature junctional assembly that bars standard cell delineation techniques based on junctional outlining. By sporadically labeling podocyte cytoplasm with a fluorescent label in vitro (by transient adenoviral or stable lentivirus transduction), the whole-cell morphology of single fluorescent cells adjacent to optically silent cells in vitro²⁷ were resolved, delineated, and quantified by fractal dimension—a technique hereafter referred to as the “Mosaic assay”.

To that end, this technique for viewing confluent branching cell morphology of cells generally comprises sporadically labeling cytoplasm of a portion of the cells with green fluorescent protein (GFP) in vitro. It should be noted that GFP is used in this example, however the present disclosure is not limited to the use of GFP. Any fluorescent label (e.g., red fluorescent protein (RFP)) may be within the scope of the present disclosure. This labeling of cytoplasms with GFP may be performed by transient adenoviral or stablelentivirus transduction.

The GFP-labelled cells are then mixed with another non-fluorescent portion of the cells. In some applications, the GFP-labelled cells and the non-fluorescent cells may be mixed at a ratio of in the range from 1:2 to 1:15. The mixed cells are then stained to visualize confluent monolayers. Among other options, the mixed cells may be stained with rhodamine-wheat germ agglutinin.

The single GFP-labelled cells that are in contact with non-fluorescent cells that are arranged in a monolayer are then identified and imaged. The monolayer may be imaged with a super resolution confocal microscope.

Fluorescent Cell Transduction for Mosaic Assay

To develop the mosaic assay, populations of fluorescent cells were mixed with optically silent cells and monitored for over 14 days in the mouse cell line, and over 5 days in the human cell line. For long-term live cell labeling, adenovirus transduction of eGFP (Vector Biolabs, #1060) was utilized at an MOI of 1000 in the mouse podocyte cell line, and MOI of 10 in the human podocyte cell line. Cells were transduced one day after seeding and attachment, when cultures were 75% confluent. Virus handling protocols were used, including wearing additional disposable PPE (lab coat, shoe covers, elbow-length and double gloves, and face mask). All virus-contacting consumables including vials, tips, serological pipettes, and gloves, were submerged in 1% bleach and allowed to decontaminate for over 30 minutes. Liquids were decontaminated in bleach where the final combined volume had 1% bleach. Virox wipes were used to disinfect surfaces, and cultures were incubated and transported in secondary containers in the lab. To generate a stable fluorescent human podocyte cell line for repeated use, 200,000 cells/mL were seeded in 6-well plates with 500 μL of lentivirus.

The next day, virus was removed and puromycin was applied at 8 μg/mL to select fluorescent cells for 48 hours. Puromycin was removed and media replaced to allow cells to recover before passaging and freezing vials. One vial was then expanded and mixed with the equivalent non-fluorescent podocytes from the same passage number, with 1/12 fluorescent cells in the mixture. The mosaic assay was assessed using the Zeiss Elyra 880 LSM super resolution confocal microscope to allow sufficient resolution to see interdigitating foot processes. Cultures were fixed in their wells at 2% PFA, stained with rhodamine-wheat germ agglutinin at a dilution of 1/500 at room temperature for 2 hours, and mounted with DAPI-containing mounting media (Vector Laboratories, Vectashield #H-1200), to visualize confluent monolayers. Only single fluorescent cells in contact with non-fluorescent cells and arranged as a monolayer (not overlapping) were imaged to use for analysis.

EV isolation and NTA: For extracellular vesicle (EV) experiments, cells were rinsed twice with PBS and incubated with serum-free culture media for 48 hours. 8 wells of cell media for each topographic platform were pooled together and collected into a 15 mL centrifuge tube. Both early time point (day 4) and late time point (day 14) samples were collected. Samples were spun at 3000 g for 10 minutes at 20° C. to remove cell debris. EVs were isolated by the miRCURY Exosome Isolation Kit (Qiagen, #76743) based on the manufacturer's protocol. Precipitation buffer was added to the samples at a 4:10 ratio, then rotated overnight in the cold room at 4° C. for EV precipitation. Precipitated EVs were spun at 3200 g for 30 minutes at 20° C. The supernatant was discarded, and the pellet was gently washed with PBS. Isolated EVs were resuspended in 100 μL resuspension buffer for downstream analysis. The size distribution of EVs was generated by Nanosight NS300. Samples were diluted in PBS to obtain the optimal detection: 60-100 particles in frame. Three 30-second videos were recorded with camera level 14. The size distribution curves were generated with the detection threshold 3.

Second harmonic generation imaging: Scaffolds were prepared as for cell culture and coated with rat tail collagen I solution at 0.1 mg/mL in PBS, for 2 hours at room temperature. The coating solution was removed, samples rinsed once with PBS, transferred to a petri dish, left wet with PBS, and covered with a cover slip for imaging. A Zeiss LSM710 Two-Photon/Confocal microscope was used with DI water as the immersion fluid on a W Plan-Apochromat 20×/1.0 DIC objective lens. Pinhole size was set to 1.247 airy units. The laser was set to 840 nm and the second harmonic generation signal was recorded at 420≅5 nm. Other signal emissions from 443-748 nm were detected as a separate noise channel for reference. Gain was set to 810.

KPFM: Kelvin Probe Force Microscopy (KPFM) was conducted on an MFP-3D AFM (Asylum Research, Oxford Instrument) using a conductive Ti/Ir coated tip (ASYELEC.01-R2, f=73.78 kHz, k=2.0 N/m). The tip was calibrated using the Sader's method^{48,49}. The KPFM measurement was implemented at a scan rate of 0.5 Hz with 256×256 pixel density in each image. The applied KPFM in the present system adopted a two-path method, where the first pass in every scan line was used to determine the topography and the following second pass was used to measure the contact potential difference between the tip and the sample. The second pass scanned by raising the tip at a fixed 30 nm above the sample following the topography.

Streaming potential: Surface zeta potentials of the samples were evaluated using a Surpass™ 3 Electrokinetic analyzer (Anton Paar, Graz, Austria). The zeta potential values were determined over the pH range of 6-8 and 25° C. using a 1 mM KCl solution based on measuring the streaming potential. The pH of the electrolyte solution was adjusted using HCl and NaOH solutions. At each pH, zeta potential measurement was repeated at least 3 times to ensure the reliability of the results. The value of the zeta potential at neutral pH would be more reliable if the zeta potential at the higher and lower pHs are also measured.

Hot Embossing: A baseplate mold was created by stitching concave PDMS topographic round inserts together with PDMS filler according to the alignment of a 24-well plate. The baseplate patterning was then transferred from the stitched PDMS mold onto a 0.05 inch thick polystyrene sheet (McMaster-Carr) via hot embossing using a Carver 3889 Heated Laboratory Press (Carver Inc.). Hot embossing was performed at a temperature of 180° C., clamp force of 4530 N, clamp pressing speed of 15%, and pressing cycle of 10 minutes. After air cooling, patterned polystyrene base plates were manually removed from the mold. Polyurethane glue was applied to a bottomless well plate and sealed to the hot-embossed polystyrene base plate.

RNA isolation: Cells were rinsed once with warm PBS prior to RNA extraction and isolation using the PicoPure RNA isolation Kit (Thermo Fisher Scientific, #KIT0204). 50 μL lysis buffer was added to each scaffold top, placed on a shaker for 5 min at room temperature, and gently scraped with a pipette tip and liquid collected while avoiding bubbles. Samples were incubated at 42° C. for 30 minutes on a heating block in microtubes. Manufacturer's protocols were followed, with the inclusion of a DNAse treatment step (Qiagen, #79254). 21 μL of elution buffer was used in the last step, and a DeNovix spectrophotometer was used to approximate sample RNA concentration and quality. Extraction and isolation were performed in the same sitting. Isolated RNA was kept frozen at −80° C.

Targeted transcriptome sequencing: Targeted whole transcriptome sequencing was done using the AmpliSeq for Illumina Transcriptome Human Gene Expression Panel (Illumina, Inc) following manufacturer's recommendations. This panel contains ˜20,000 genes that are assayed to expression analysis. The samples were sequenced on an Illumina NovoSeq 6000 system using 75 bp paired-end reads on either the S1 200 cycle or SP 200 cycle flowcells.

Model virus infection: Following a wash step with sterile PBS, cells were treated with concentration of virus equivalent to MOI 0.1 (for 1/100 dilution of NL63) and MOI 0.2 (1/100 dilution of 229E) in IMEM media (if 10⁵ cells were plated on the platform). Cells were exposed to virus at 34° C. for 1 hour or 10 minutes (the temperature that is ideal for seasonal corona viruses to bind to cell membrane), then virus-containing media was replaced by regular podocyte media. Cells at this point were incubated at 37° C. until they were fixed after PBS washing step with 4% PFA for 30 min at RT.

Patient Serum Application

Retrospective patient serum study inclusion criteria: Kidney transplant patients with a biopsy-proven native kidney diagnosis of FSGS were included. From all patients who were included in the study, data were extracted from their medical records from the in-center database, the Comprehensive Renal Transplant Research Information System (CoReTRIS)⁵⁷. Histopathological evaluation of the kidney biopsy was performed and available for all patients. Recurrence of FSGS was defined as the histopathological diagnosis of FSGS in allograft kidney biopsies from patients whose primary kidney disease was FSGS. Allograft kidney biopsy date with FSGS diagnosis was considered the date of recurrence. A total of 6 kidney transplant patients with FSGS were included. Patients' characteristics and recurrence status (recurrent/nonrecurrent FSGS) are shown in Table 1. As expected, proteinuria in patients with early recurrence of FSGS was higher than in patients with no recurrence or late recurrence, suggestive of a circulating factor causing widespread podocyte disease. None of the patients analyzed had a prior kidney transplant, donor-specific antibodies at the time of transplant, or native nephrectomy. The median posttransplant follow-up was 6.3 years. Graft failure was not identified during the follow-up period.

The study using patient serum samples was approved by the University Health Network (UHN) Research Ethics Board (REB) study CAPCR #1805605. Samples were leveraged from the MOT biobank and histocompatibility (HLA) laboratory in a retrospective study from a subset of patients who underwent kidney transplant and experienced either recurrence or no recurrence. Samples from patients who received a kidney transplant with FSGS as a cause of ESRD were pursued. The study was retrospective and used samples that were already consented by patients for use in research. Only de-identified information was kept on file to protect personal health information. Six samples were acquired from six patients (one sample per patient). Stably GFP-transfected human podocytes were used in serum experiments, by replacing serum content in the culture media with patient-derived serum at 20%. Cells were incubated for 3-, 24-, and 48-hour time points. Serum-free and FBS-containing groups were used as control cultures.

Analysis and Statistics

PRISM 9 was used for statistical analysis by t-test (two-tailed), one-way ANOVA, or two-way ANOVA, with significance set to 5%, and multiple comparisons performed using Fisher's LSD test. Kolmogorov-Smirnov test was used to compare distributions from NTA. Normality of distribution and equality of variance were checked. Sample replicates for given experiments are specified in figure captions.

Mosaic Assay Data Analysis

Images of single fluorescent cells from the mosaic assay (used in topography, model virus, Adriamycin, and patient serum experiments) were first processed using Zeiss's ZenDesk software to facilitate delineation of whole single cell morphology as follows: the FITC channel was adjusted by setting gamma to 2.5 and adjusting the white point to 30. The DAPI channel's white point was also adjusted to 80 to facilitate counting of nuclei. In Photoshop, the magic wand tool was used to select single fluorescent cells, create binary masks, and export images of single binary cells. The sequence was recorded as a macro to facilitate automation and thus performed blindly and equivalently to all sample images. Singularity of cells were double checked by overlaying nuclei with the cell images to ensure only 1 nucleus was contained in the cell region. Binary images were outlined in ImageJ, then processed for fractal quantification in a blinded/randomized manner using the FracLac plugin, with the background locked to white. The standard box-counting algorithm was used, and digits were significant up to 2 decimal places. Data was copied to excel, unblinded, then transferred to PRISM 9 for statistical analysis and graphing.

Cells from the mosaic assay were manually counted for their formation of process “arms” and branching, as primary, secondary, tertiary and quaternary bifurcations. A circle was established around nucleus in the cell body area, then primary bifurcations were counted. From the counted primary bifurcations, secondary bifurcations were counted, etc. where there were no bifurcations at a given level, 0 was recorded in the excel analysis sheet. SEM foot process quantification was measured manually in ImageJ using the segmented line tool. A boundary length perpendicular to protruding processes was first measured, then the same boundary was measured again but following the perimeter around protruding processes. The number of protruding processes within the boundary length were counted. The data were recorded in excel to extract the digitation ratio and to approximate the average process length and density.

Targeted transcriptome analysis: Samples were sequenced between 8 and 16 million reads (average 11 million). The raw reads in FASTQ format were aligned to the human hg19 reference genome using bwa mem with default parameterso⁵⁰⁻⁵². The reads were quantified using htseq-count⁵³ and a custom targeted transcriptome annotation GTF file provided by the manufacturer. All samples passed post alignment and quantification QC. DESeq2⁵⁴ was used for differential gene expression. Differential expression was compared between HF vs. NT; RT vs. NT and HF vs. RT groups after adjusting for the technical replicates in each group. Only genes with 10 or more reads (median) were included in the analysis. The significant genes were filtered using a false discovery rate of 5% or less. Pathway enrichment and causal network analysis was done using Qiagen's Ingenuity Pathway Analysis software. The kmeans clustering module of the R package pheatmap⁵⁵ was used to stratify the list of podocytes enriched gene list from Park et al.³² of genes into four distinct clusters based on their expression profiles. ShinyGo⁵⁶ was used on the members of these four clusters to identify their functional role and categorization.

Results

When collagen I was deposited on the miCRAFT scaffolds to facilitate cell attachment, second harmonic generation imaging revealed a distinct alignment of collagen fibrils along regions of scaffold curvature (FIG. 2e). Since collagen I is known to be slightly positively charged in neutral solutions^{20,21}, and generally hydrophobic PDMS presents a slightly negative surface charge under aqueous conditions (FIG. 2f), it was sought to be determined whether electrical properties may influence molecular patterning in addition to the steric effects, where increased presence of deposited molecules may simply be related to a higher available surface area and curvature. In FIG. 2f, flat (NT) and high-fractal (HF) topographic substrates were measured by electrokinetic streaming potential from pH 4 to 9. N=1 scaffold measured 3-5 times at each pH. Data shown as average±SD. Kelvin probe force microscopy (KPFM) suggested a shift in surface potential on the different scaffold regions, where convex curvature was found to have more negative charge compared to flat regions (FIG. 2g). Further, different degrees of surface curvature affected the measured zeta potential of the material in bulk (FIG. 2h).

Although theoretical derivations of curvature-charge density relationships for conductors are well known, such relationships are not well understood for dielectric materials, such as PDMS, or insulators²². The nonlinear zeta potential trend with scaffold complexity measured may suggest that a higher degree of negative Gaussian curvature may counteract the higher degree of low-radius positive Gaussian curvature, as seen in the HF scaffold, behaviour which may be associated with the mobility and hydrophobic recovery properties of PDMS moieties, and with differential adsorption of counterions to the curved and higher surface area topographies compared to flat. These data point to a curvature-induced charge clustering (CICC) mechanism that facilitates patterning of extracellular matrix (ECM) molecules on fractal PDMS scaffolds in addition to simply providing a higher surface area, where the vectors for electrostatic repulsion between charged molecules are dispersed in more directions on convex curvature, thus facilitating higher molecular packing than in flat configurations (FIG. 2i).

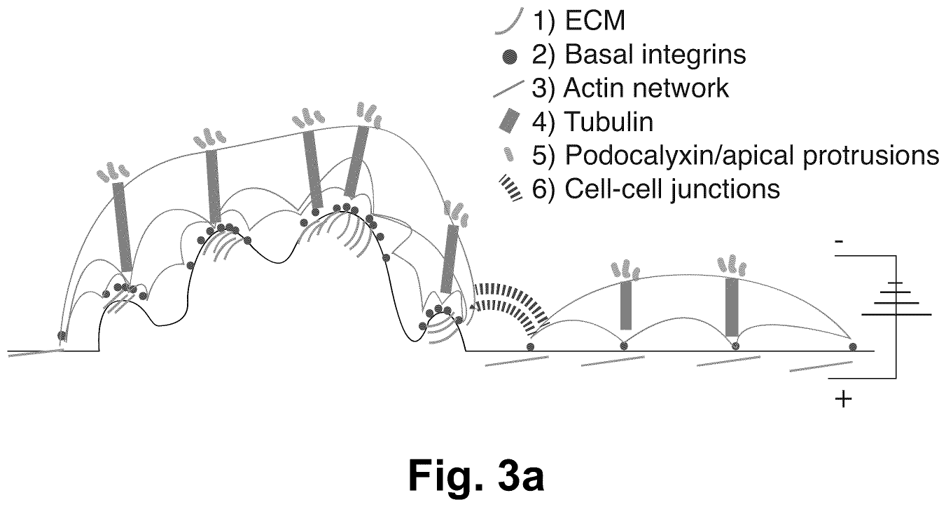

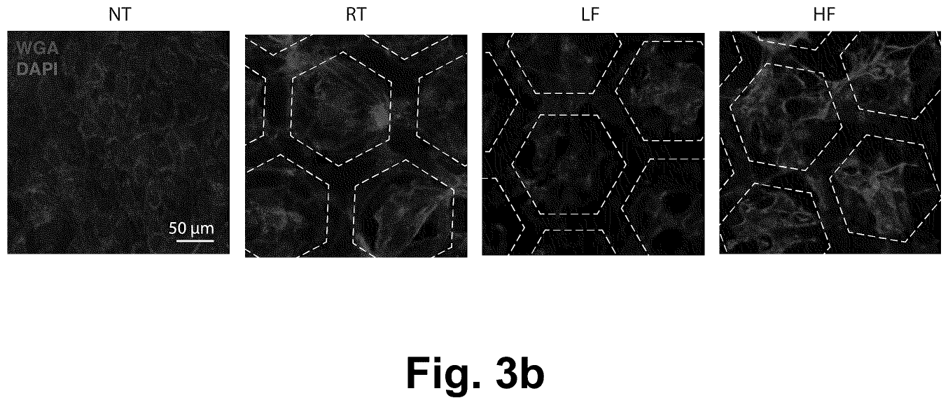

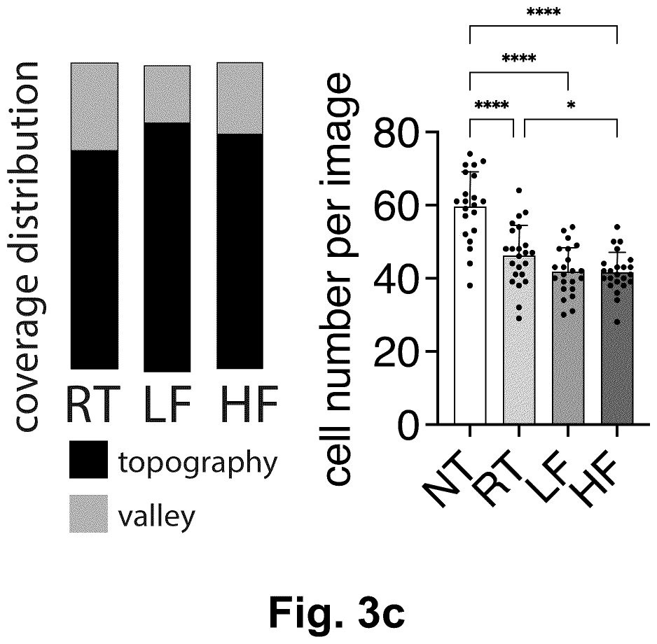

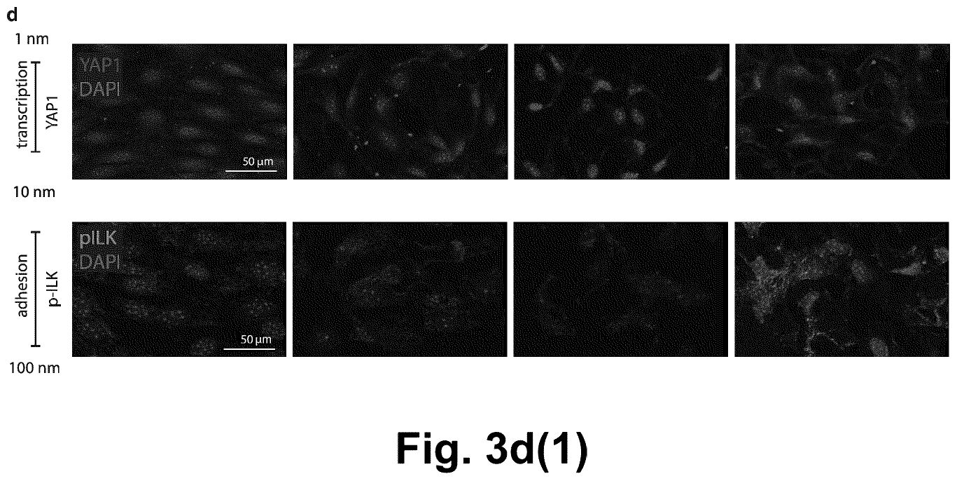

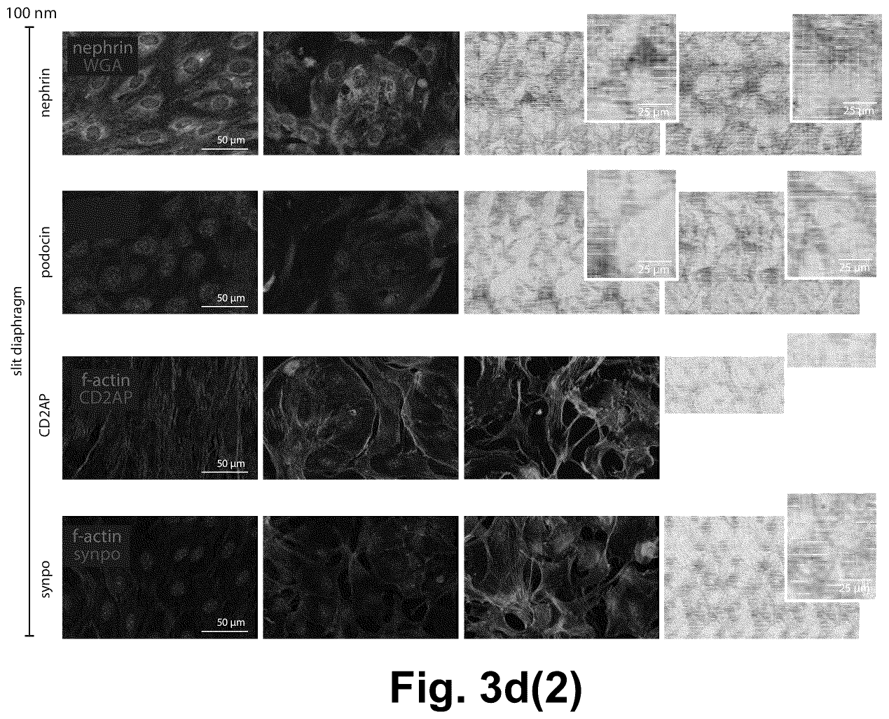

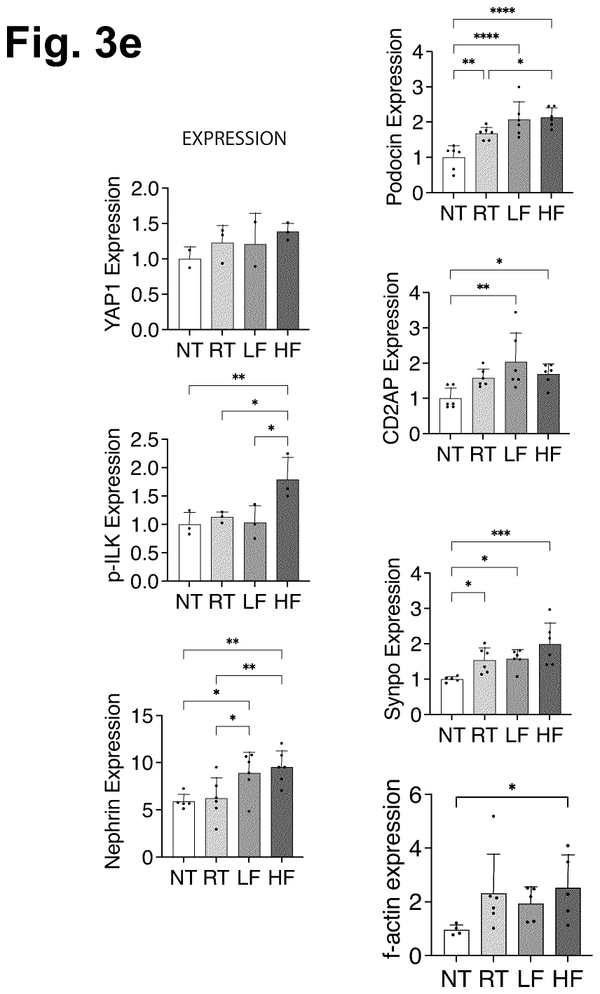

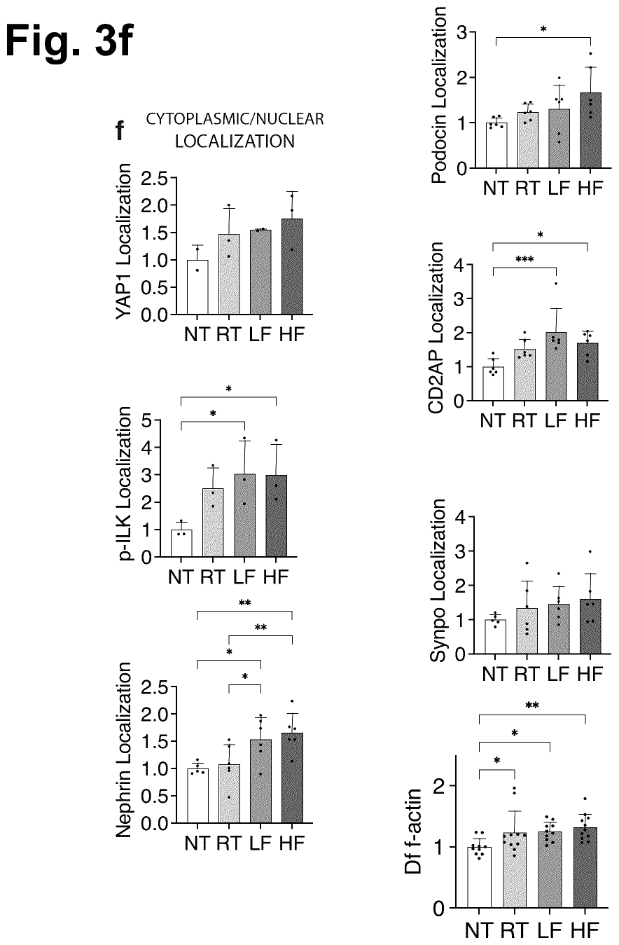

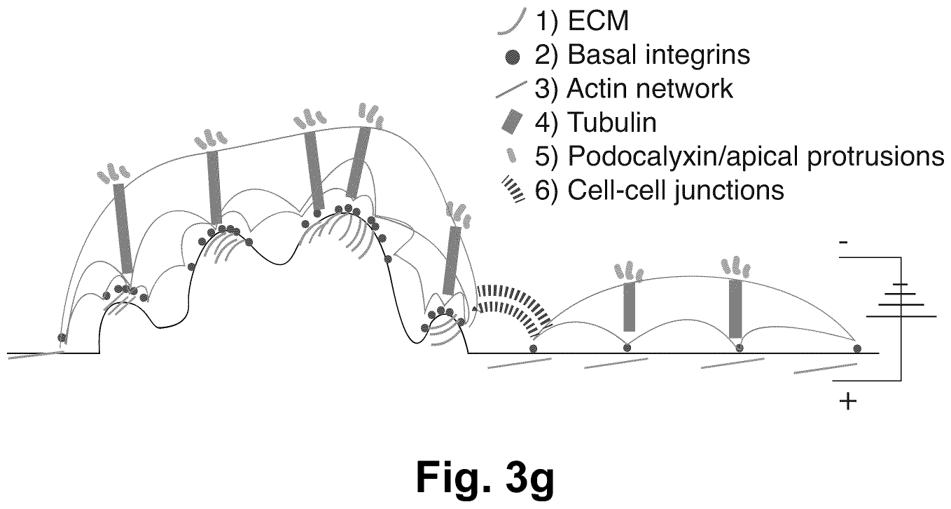

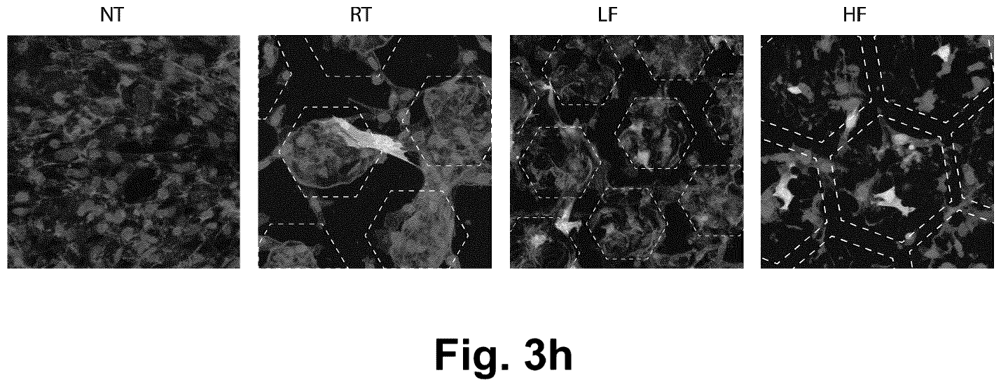

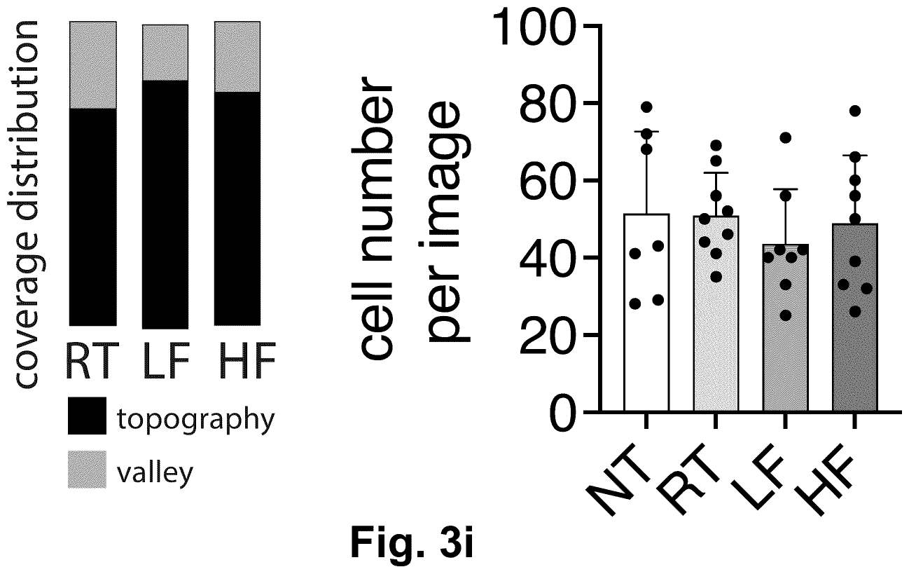

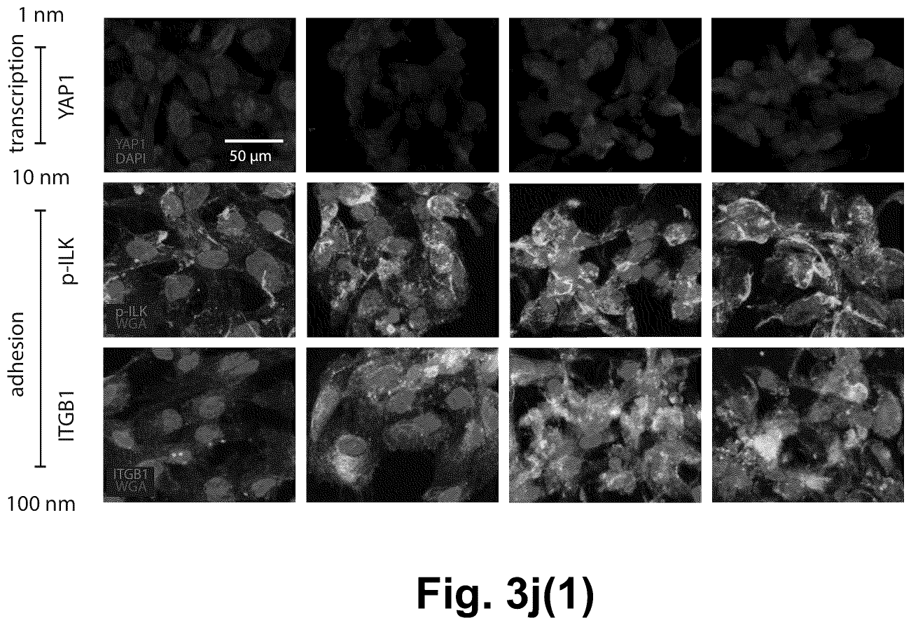

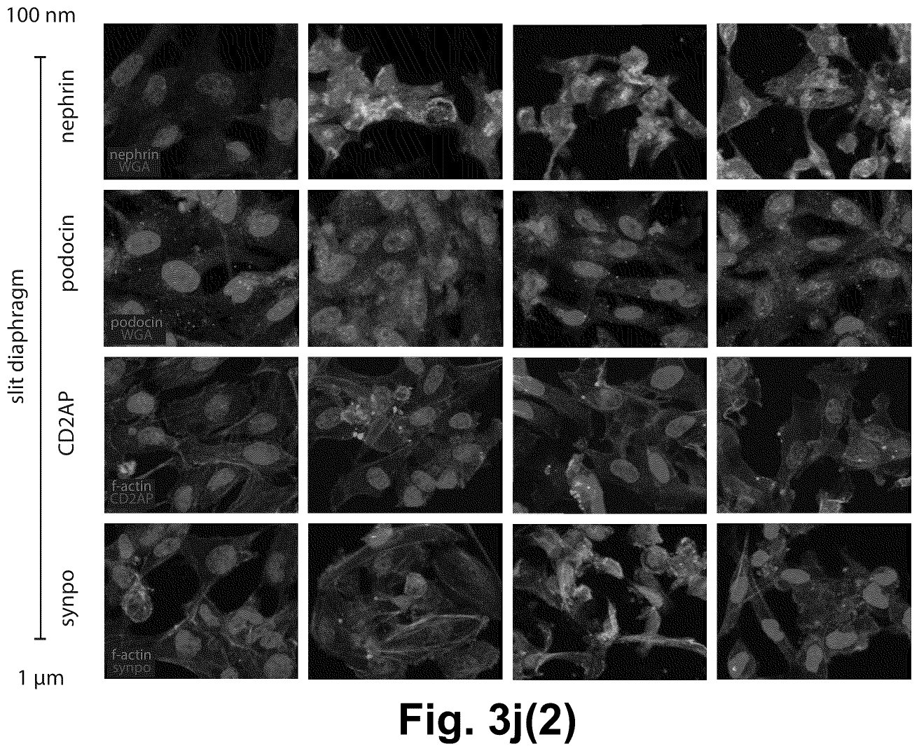

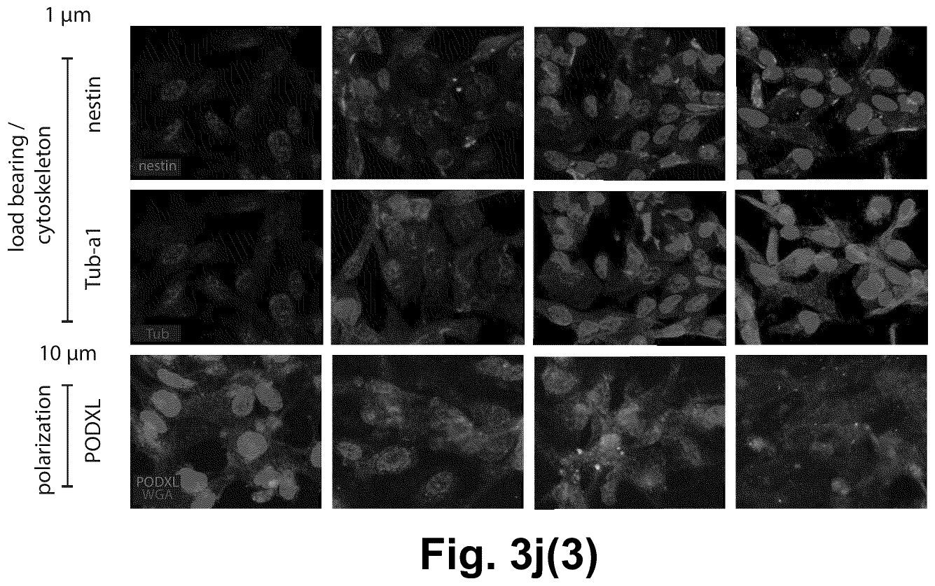

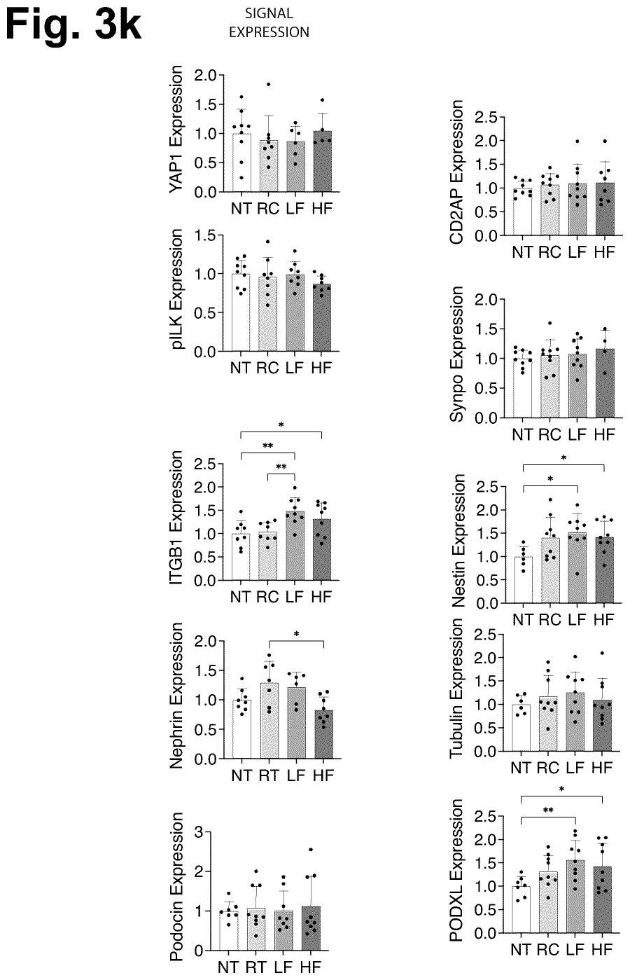

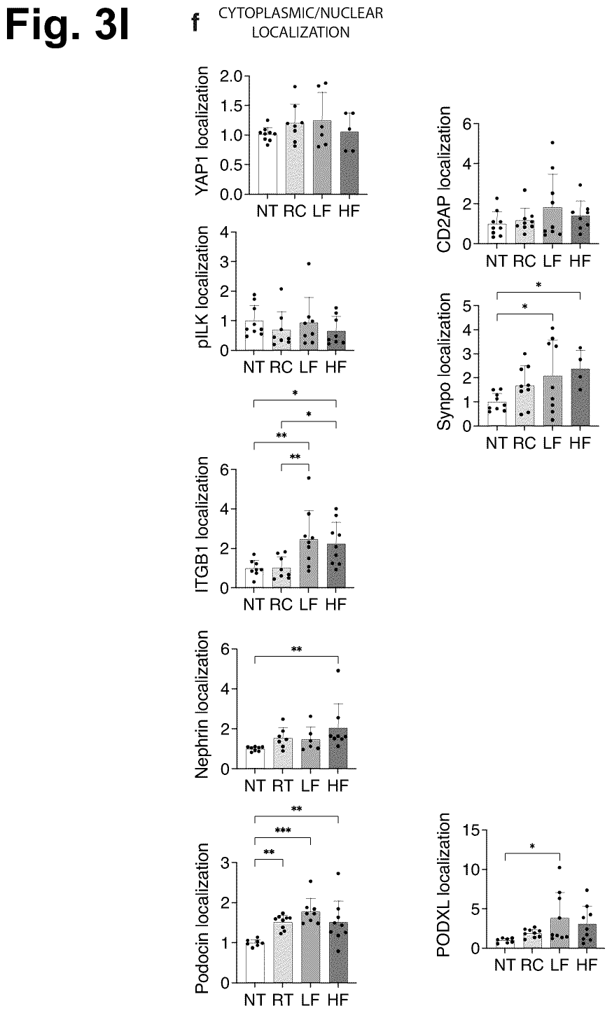

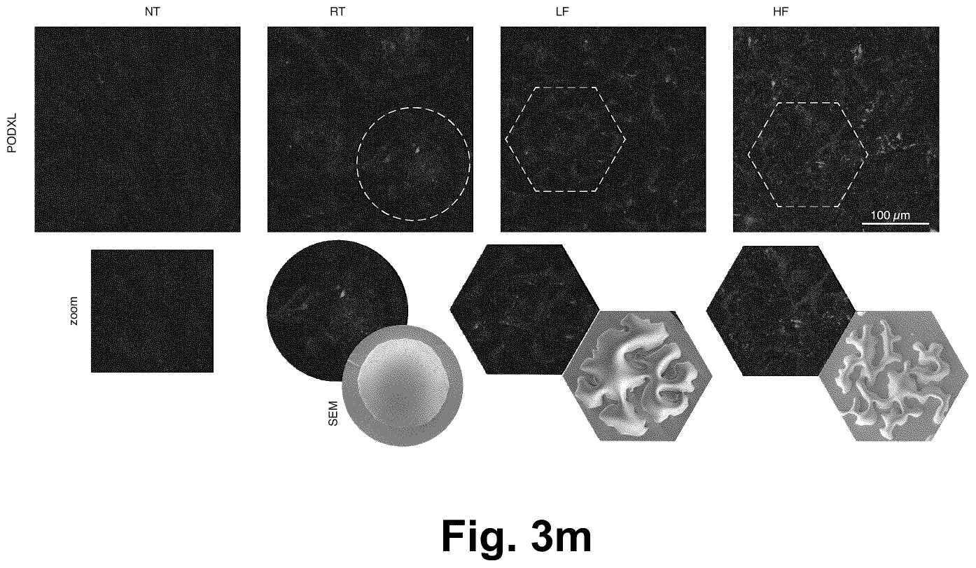

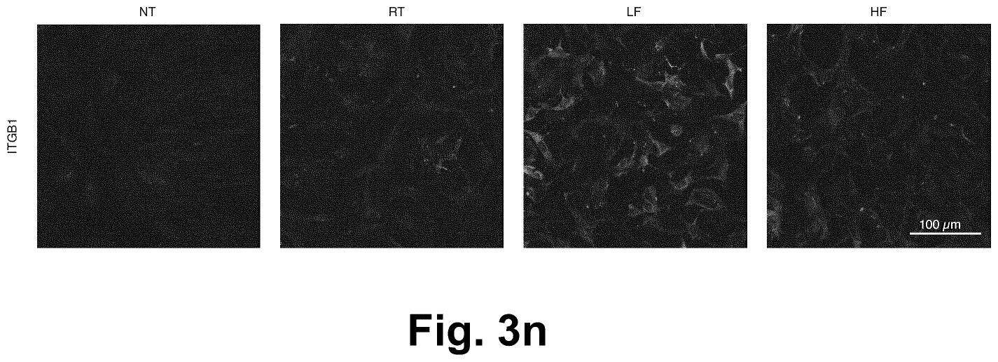

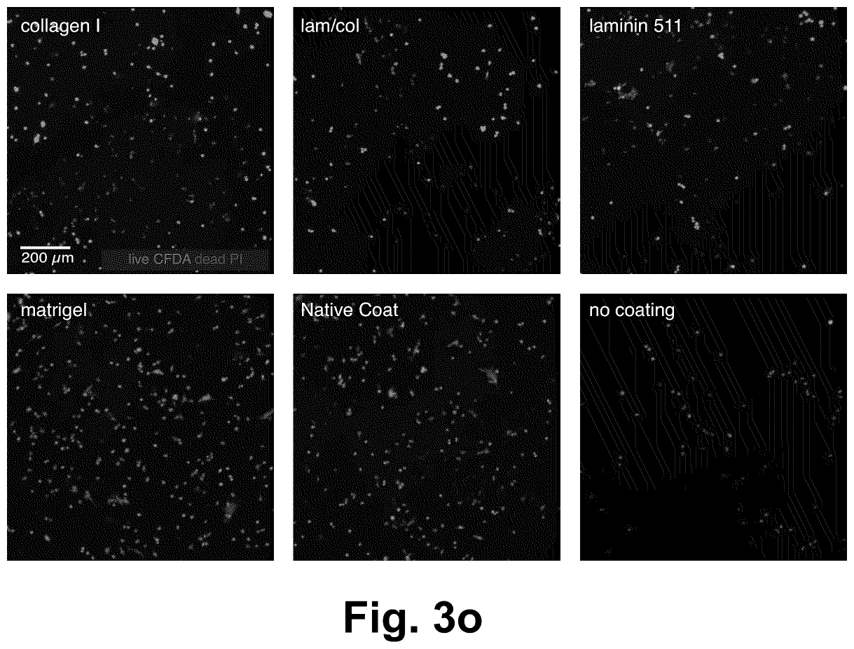

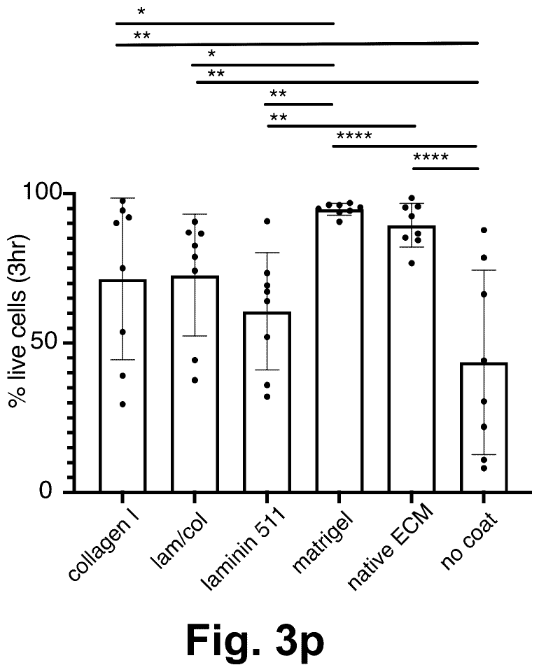

Apical-basal polarity is established in epithelial cells when multiple structures are stacked together in a coordinated fashion (FIG. 3a). It was postulated that the arrangement of ECM proteins, facilitated by fractal shape cues, could mediate multiscale assembly of cell structures involved in the polarization phenotype typical of maturing podocytes. Podocytes (both mouse and human cell lines) grown on 2½-D surfaces were evaluated using immunofluorescent staining and confocal maximum intensity projection imaging. Podocytes grew on the curved “slices” of patterned scaffolds in island-like distributions (FIG. 3b), and had reduced cell density compared to NT (FIG. 3c), likely due to attenuated proliferation²³ due to physical boundaries and lack of attachment on valleys between features within the topographic scaffolds, supporting an overall preference for cells to reside on topography.

By immunofluorescent staining of key podocyte and polarity-related markers, validated with both mouse (FIGS. 3a-f) and human (FIGS. 3g-l) podocytes, a dose-response-like trend in expression and localization with extent of topography was generally observed, accompanied with formation of intense clusters on “peak” regions of the fractal scaffolds (FIGS. 3d(1)-(2) and j(1)-(3)). Specific markers were observed at relevant length scales. YAP1 is a transcription factor associated with the HIPPO pathway that modulates tissue growth depending on localization: in the nucleus, tissue expansion is activated; in the cytoplasm, growth is deactivated and maturation is favored instead. Stable YAP1 expression was observed in all scaffold groups (FIGS. 3e and 3k) with transfer from nuclear to cytoplasmic localization as topography increased (FIG. 3f), consistent with cell density results suggesting a shift towards maturation. Integrin ß-1 and integrin-linked kinase expression and localization increased on topography, suggesting binding and signaling activity with the ECM, also consistent with observed ECM patterning and cell attachment. With scale up, enhanced expression and localization were found in essential slit diaphragm proteins: nephrin and podocin, involved in the intracellular bridge, and synaptopodin and CD2AP, involved in actin stabilization (FIGS. 3d(1)-(2) and j(1)-(3)). Nestin, an intermediate filament that provides tensional load-bearing and structure in major podocyte processes²⁴, was also found to increase in fractal and topographic groups compared to NT, suggesting differential force distribution and major branch formation. Finally, podocalyxin, a negatively charged sialylated glycoprotein involved in polarization²⁵ and basal organization²⁶, exhibited enhanced expression and organization according to fractal topography as well.

The immunofluorescence staining results collectively suggest an interplay of fractal cues with protein expression and organization across the spectrum of cellular machinery observed, supporting hierarchical assembly concomitant with maturation. Alignment of scaffold patterns in brightfield-fluorescence overlay images (FIGS. 3m and 3n), as well as preferential cell attachment and survival on heterogeneous ECM coatings compared to single-protein coatings (FIGS. 3o and 3p), further supports the assertion that complexity in the native milieu is advantageous for cell maturation in culture.

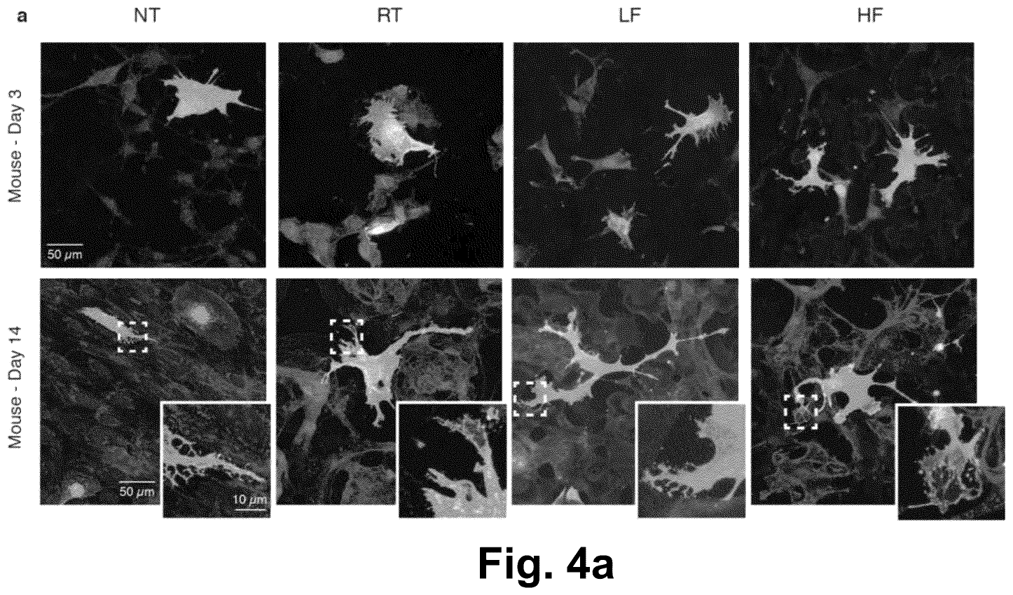

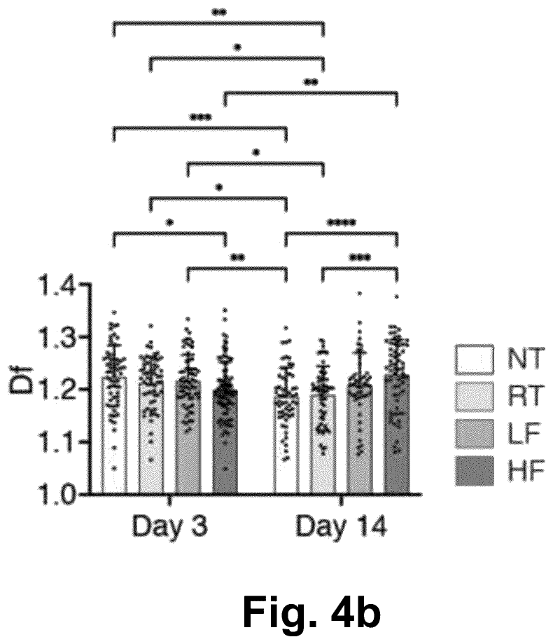

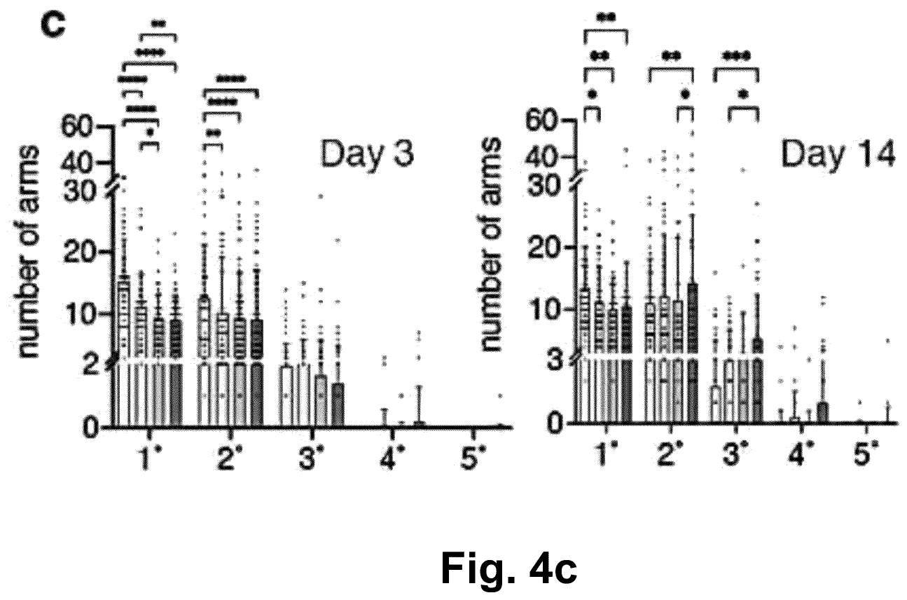

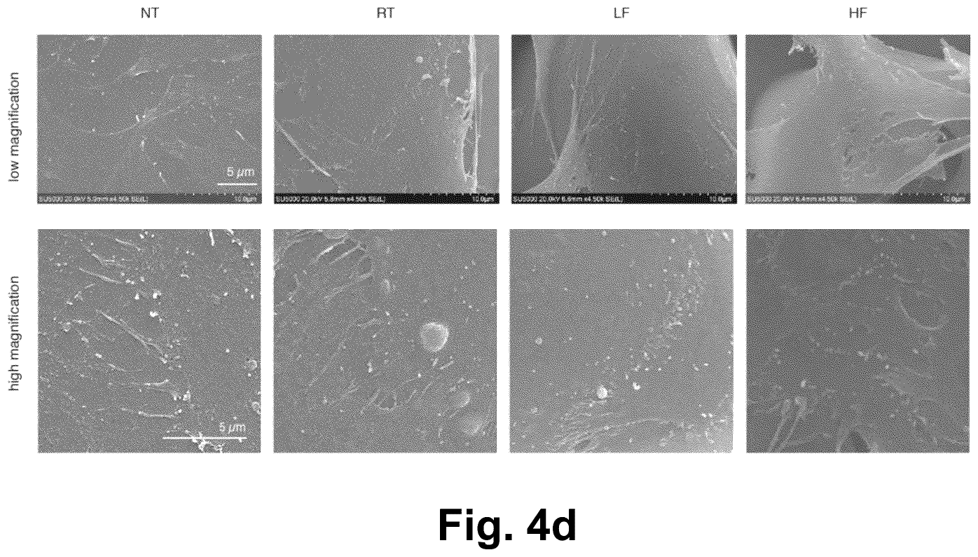

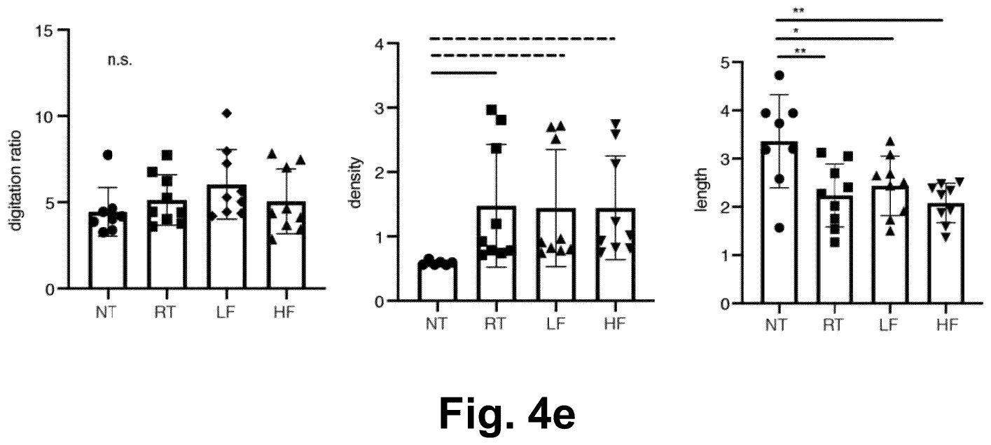

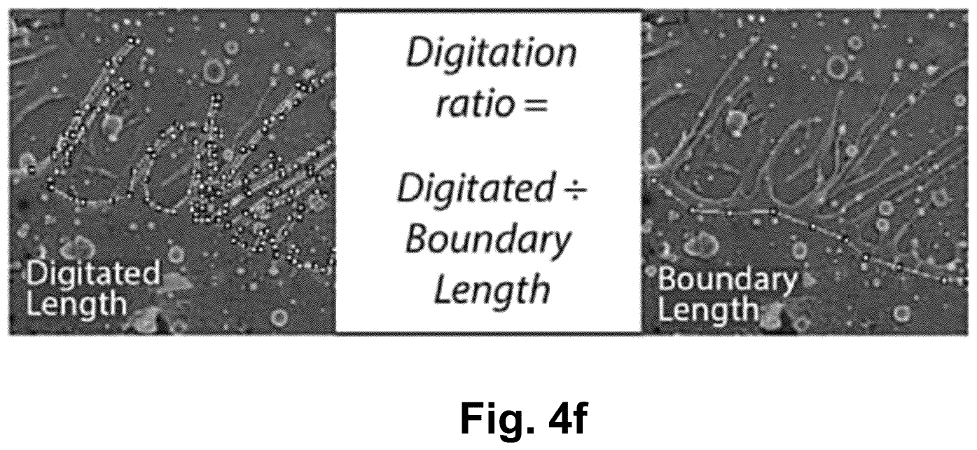

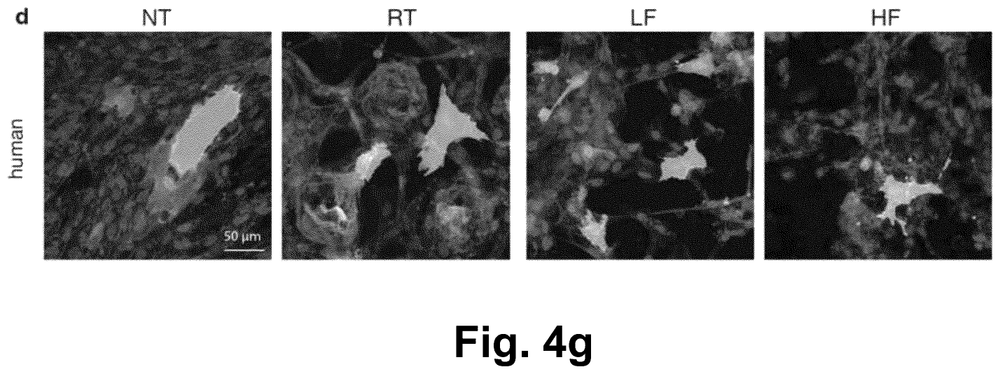

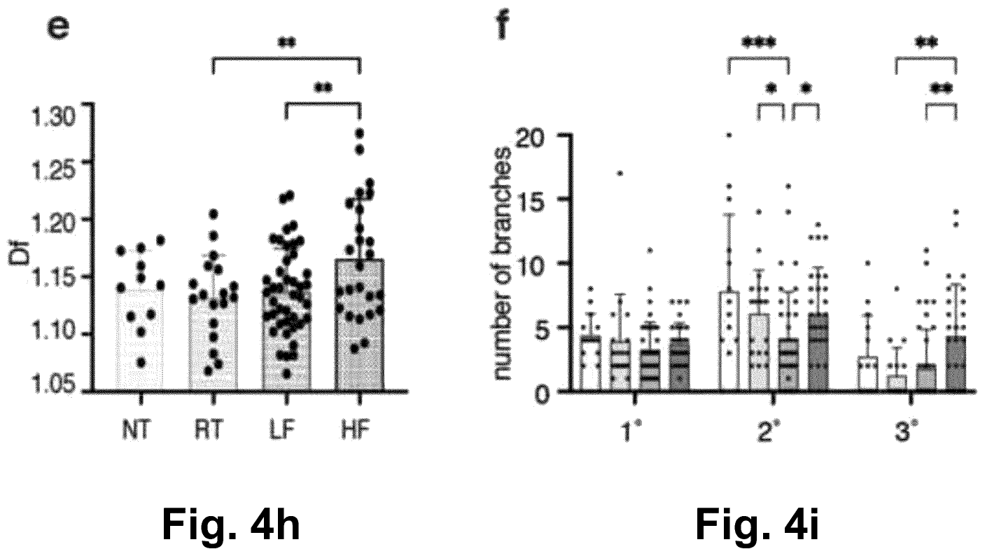

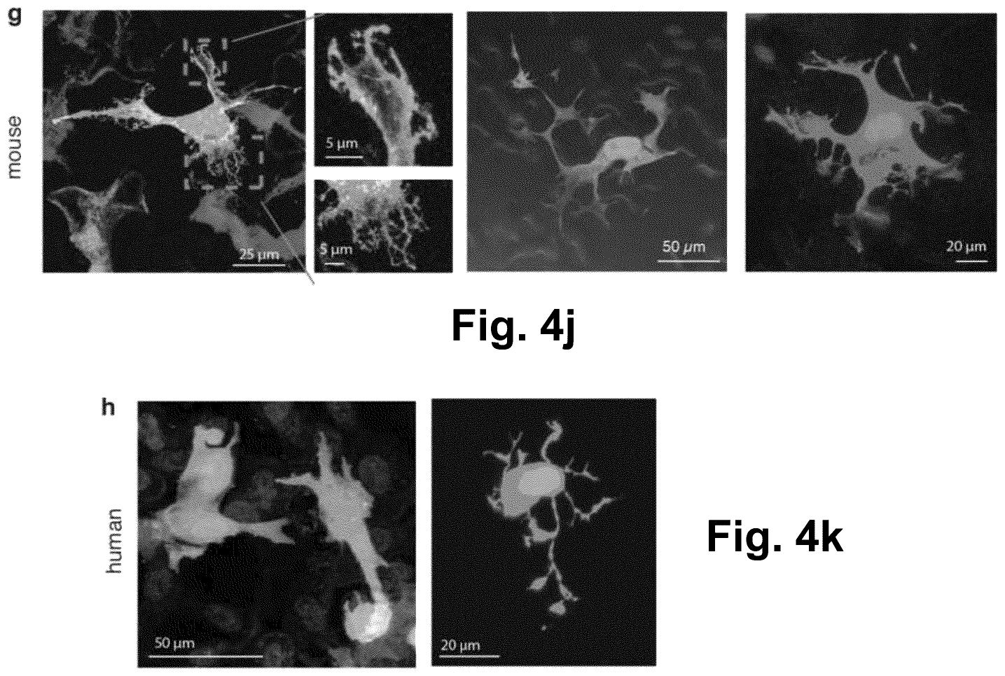

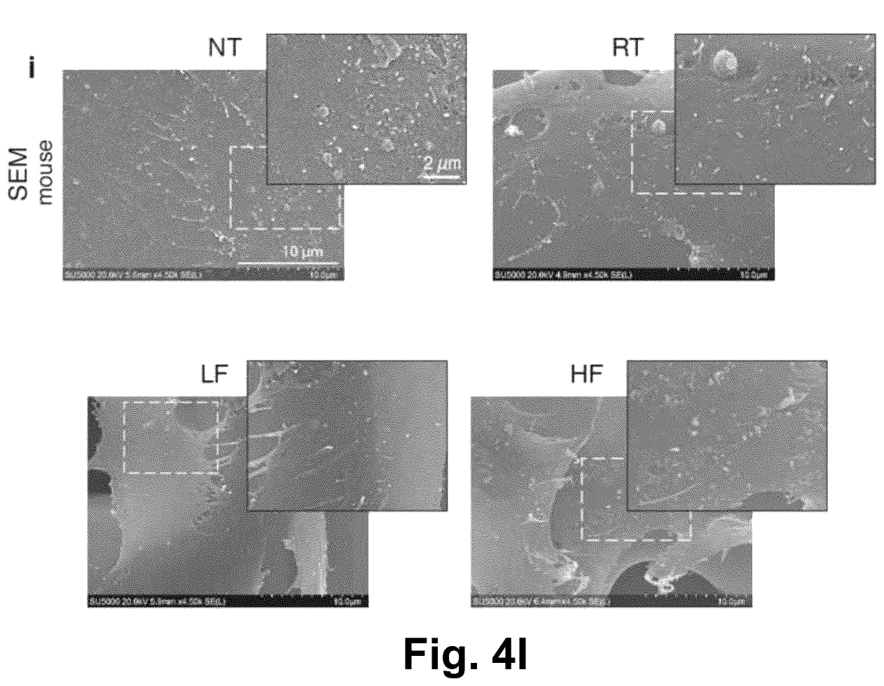

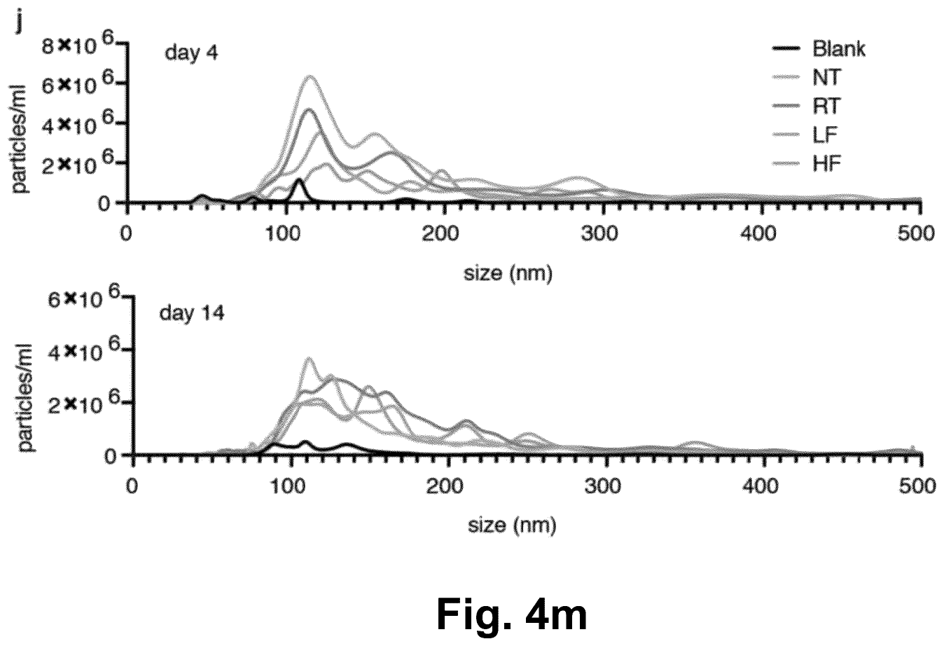

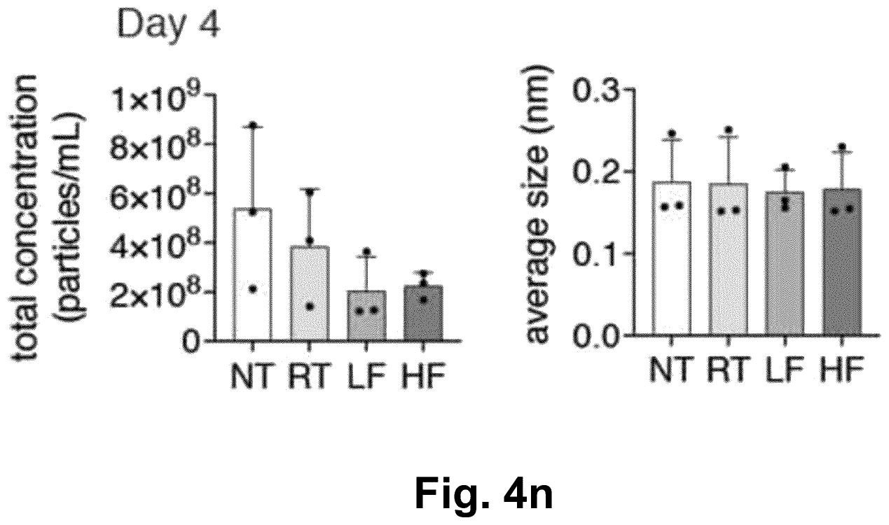

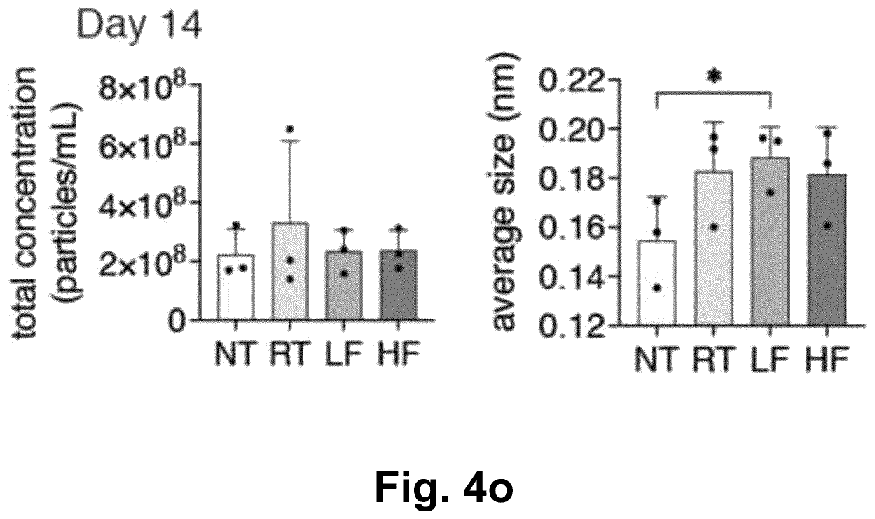

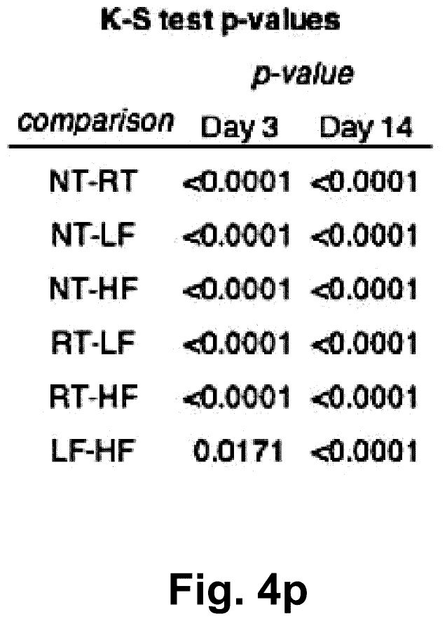

Confocal superresolution microscopy of the conditionally immortalized mouse cell line cultivated in the Mosaic assay on the four scaffolds revealed the first, clearly and truly interdigitating whole podocyte morphology established in vitro (FIG. 4a) that evolved over 14 days of cell culture. As cells became confluent and interdigitated (FIG. 4b): non-fractal groups (NT, RT) demonstrated declining cell fractal morphology over time; the low fractal topography (LF) had no change; and HF, the pattern derived from healthy histology, was the only group to experience apparent maturation of morphology with time in culture consistent with developmental progression (see FIG. 1d). By the maturation time point when proliferation is considered according to protocol to be fully switched off in the cell line, HF cells had a significantly more complex morphology than non-fractal groups in a dose-like response trend with the extent of topographic stimulation. In addition, manual quantification of the number and degree of branching processes from Mosaic cells (FIG. 4c), as well as evaluation of sample cell-cell boundaries by SEM (FIGS. 4d-f), also confirmed increased process density and convergence of process length consistent with the development and increasing morphological complexity^{4,28}.

Cultivating the Mosaic assay with a female immortalized human cell line (FIG. 4g), also demonstrated increasing morphological complexity in response to HF topography, confirmed by fractal dimension (FIG. 4h) and arm branching quantification (FIG. 4i). At confluence, the human cell line cells had overall lower extents of fractal morphology than mouse cells, which may reflect a consequence of non-conditional immortalization, highlighting the importance of cell source in generating engineered tissue models.

Overall, the Mosaic assay succeeded in revealing various cell examples in the fractal topographic groups that exhibited complex branching and evidence of interdigitating foot processes, in both mouse (FIG. 4j) and human (FIG. 4k) cell lines. By static shape stimulation only, the equivalent of about ¼ stage of developmental maturation⁴ was achieved in the HF group, supporting that fractality of glomerular looping may synergize with fractality of the cells it harbours, and that Mosaic morphology as a readout may provide an objective metric of maturation extent with cross-referencing capability (FIG. 1d).

Fractality elicits signs of glycocalyx development and activity: Apical surfaces of cells are decorated with glycans that contribute to the regulation of cell structure, function, signaling, and communication via protruding forms such as charged villi, blebs, and secreted extracellular vesicles (EVs)²⁹. Scanning electron microscopy (SEM) showed the presence of various apical membrane protrusions sprouting from mouse cells on the four scaffold groups (FIG. 4l). Using nanoparticle tracking analysis (NTA) at early and late time points, the size distribution of secreted EVs was quantified from the cultivated cell's supernatant (FIG. 4m). At the early time point, all scaffold groups secreted on average the same size of particles, corresponding to approximately 150 nm, although NT and RT secreted a higher total amount than LF and HF (FIG. 4n). Over time, the secretions from all groups equalized in amount, but changed in average size, with topographic groups secreting larger particles (FIG. 4o). The Kolmogorov-Smirnov statistical test³⁰ further confirmed distinct secretion patterns from each scaffold (FIG. 4p). Collectively, these observations point to the signs of podocyte maturation since microvesicle generation generally associates with mucin-containing glycocalyx development^{29,31}.

Distinct gene expression profiles suggest ECM manipulation is a key podocyte maturation driver by patterned shape cues. To characterize the responses of topographically stimulated cells in detail, next-generation total mRNA sequencing of primary fetal human podocytes cultivated on the scaffolds up to confluence was performed, which took three days in culture. To simplify results, only HF was included in analyses as a representative of the fractal topography group, alongside RT as the non-fractal control, and NT as the flat no topography control. Data were uploaded to the GEO database, accession number GSE185491.

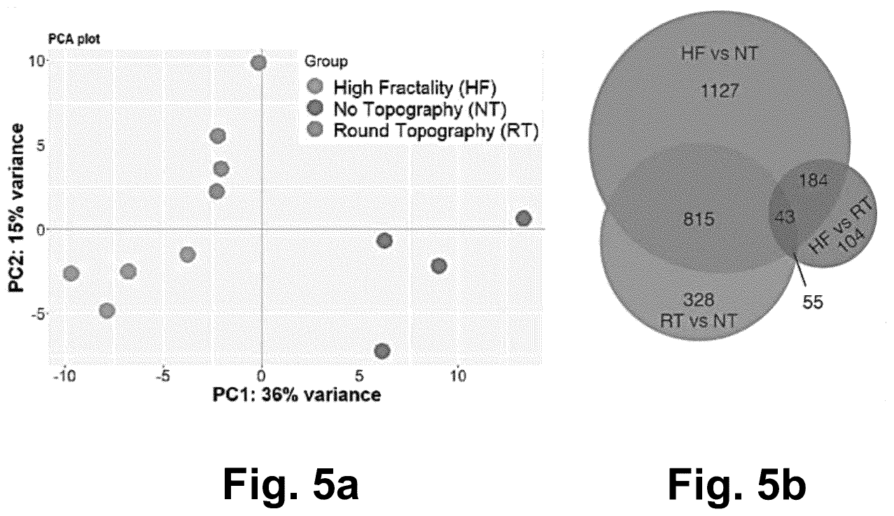

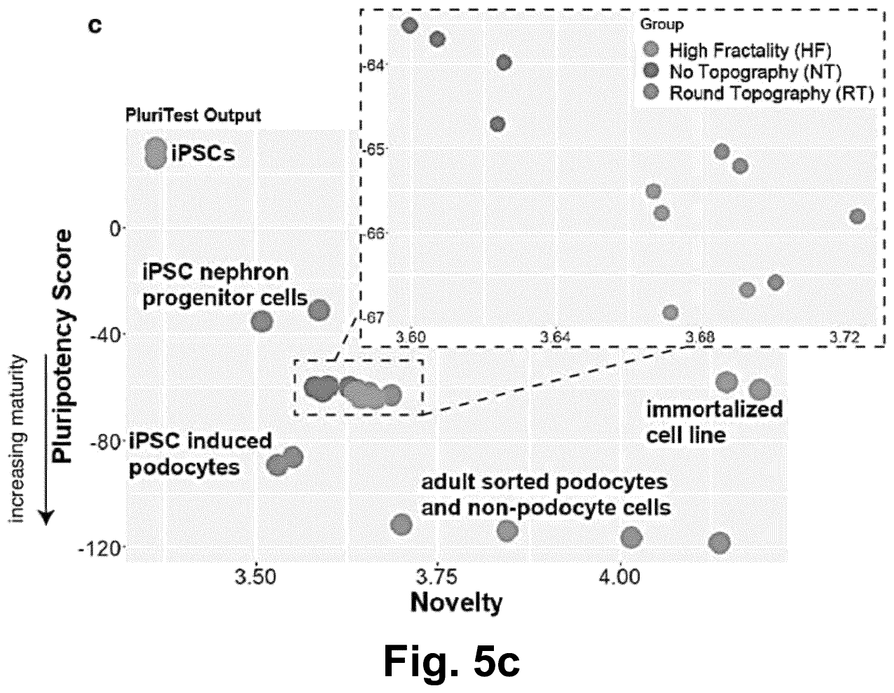

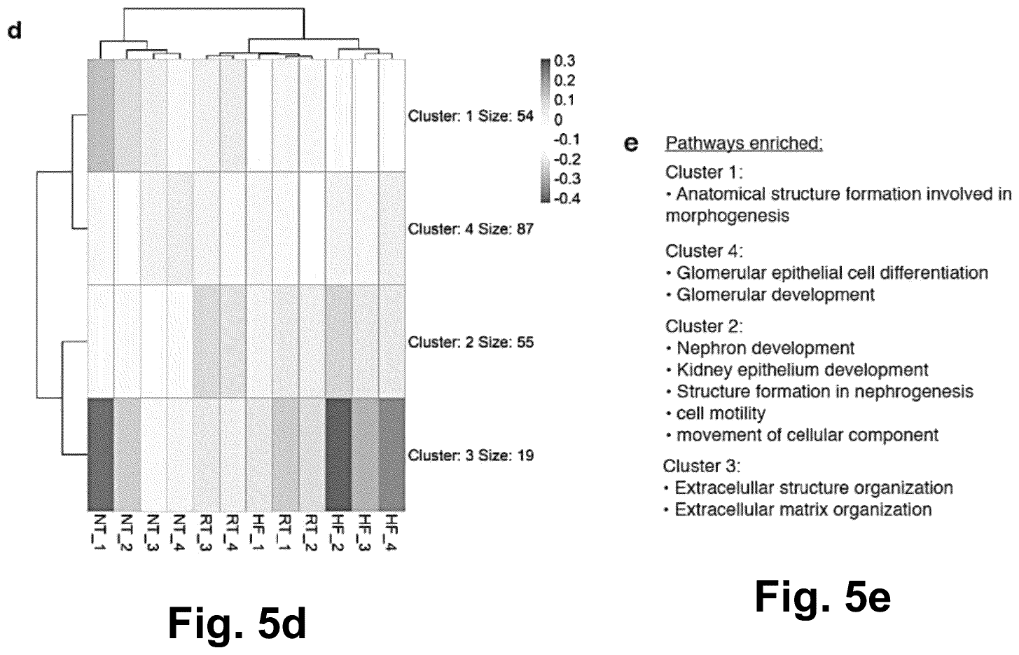

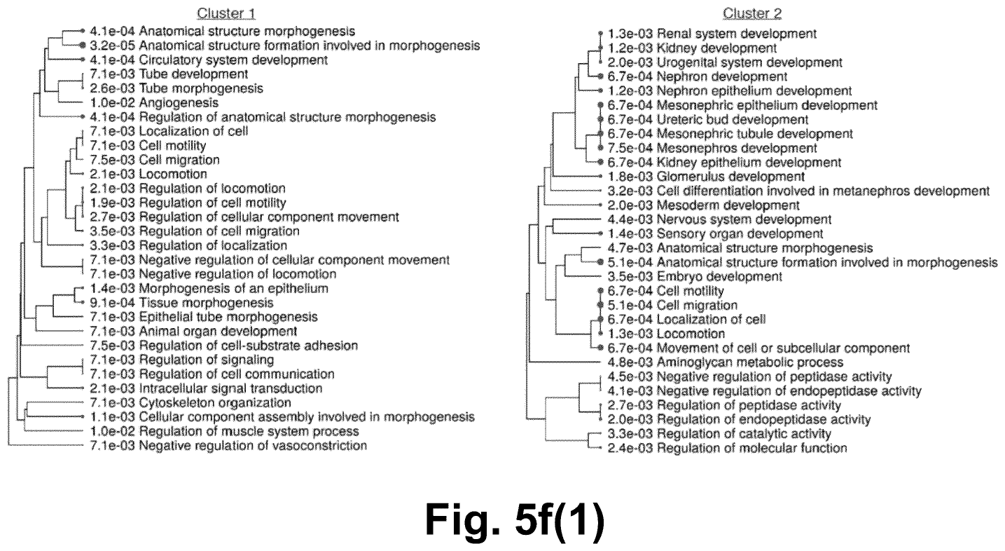

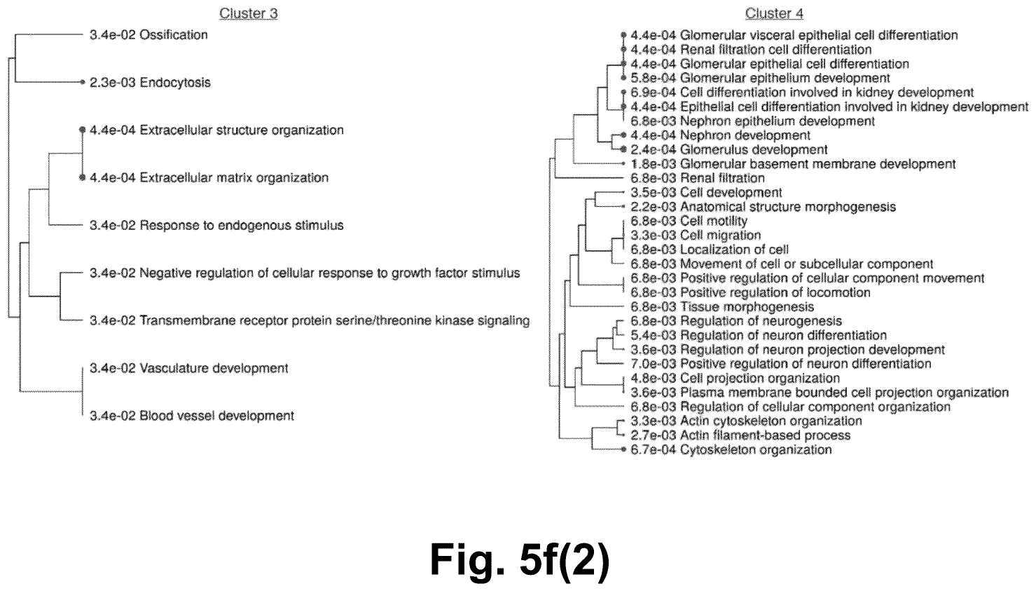

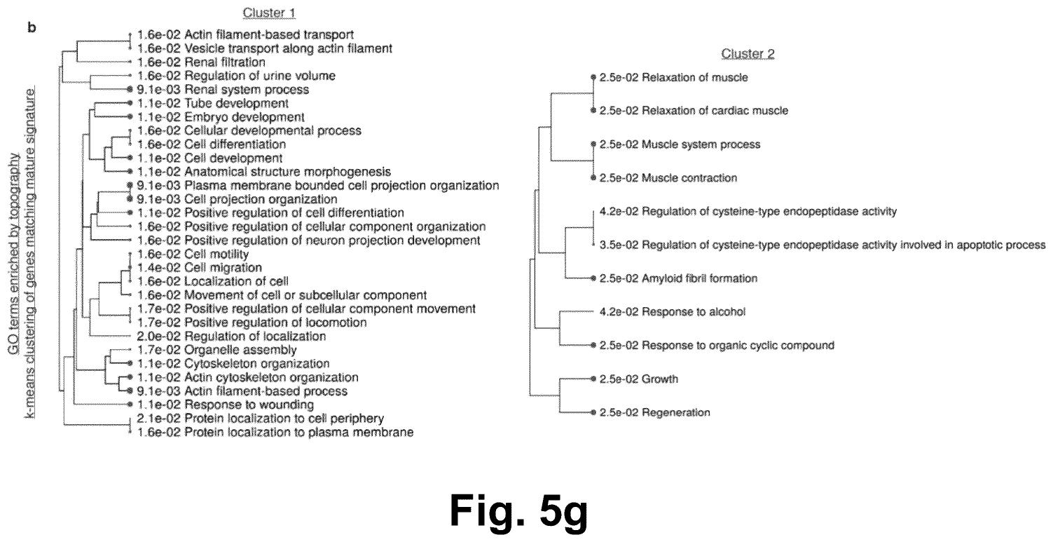

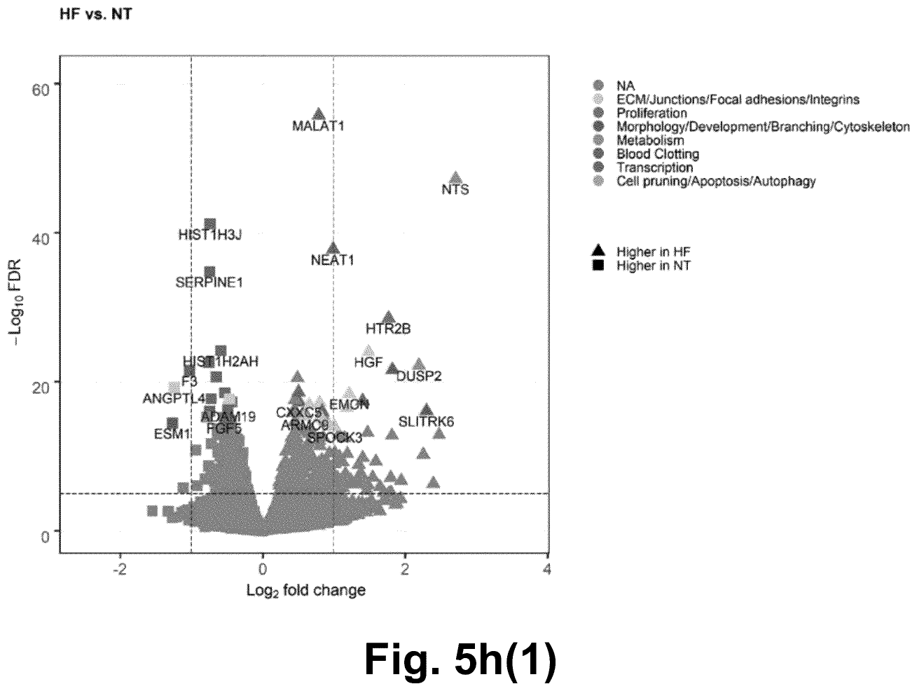

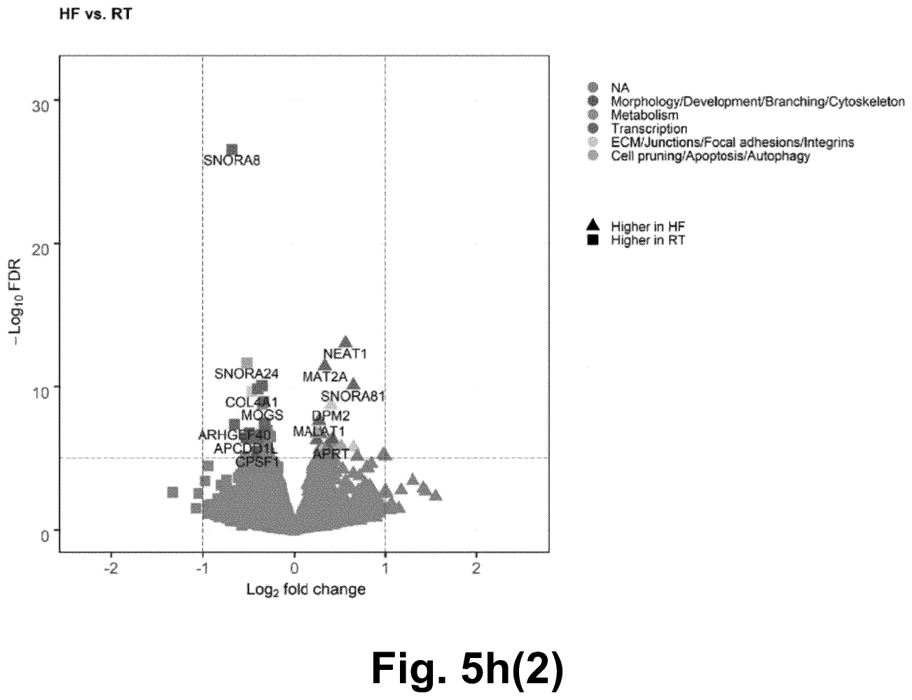

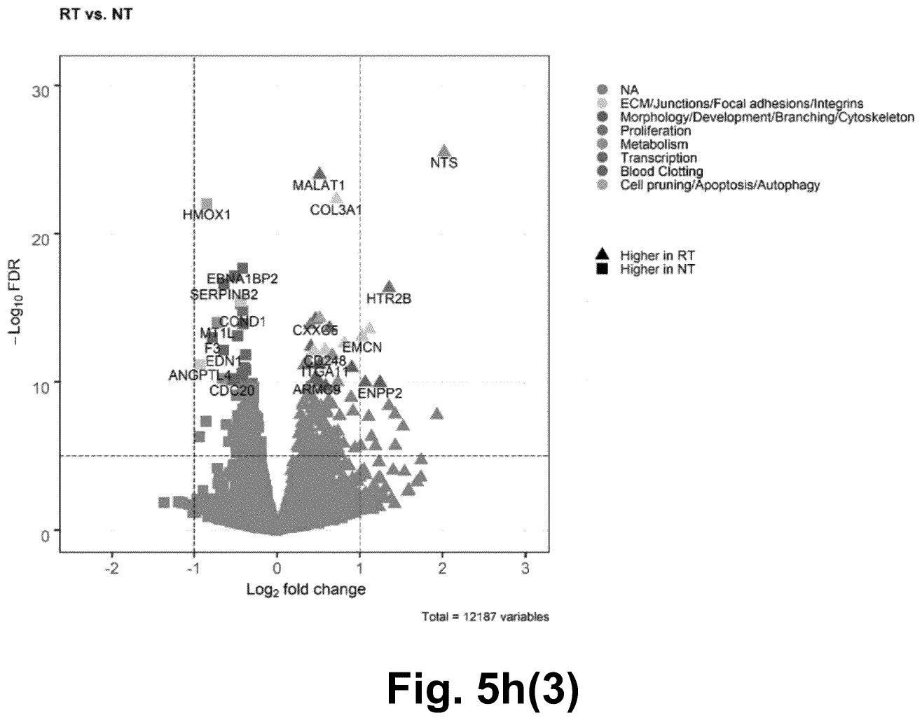

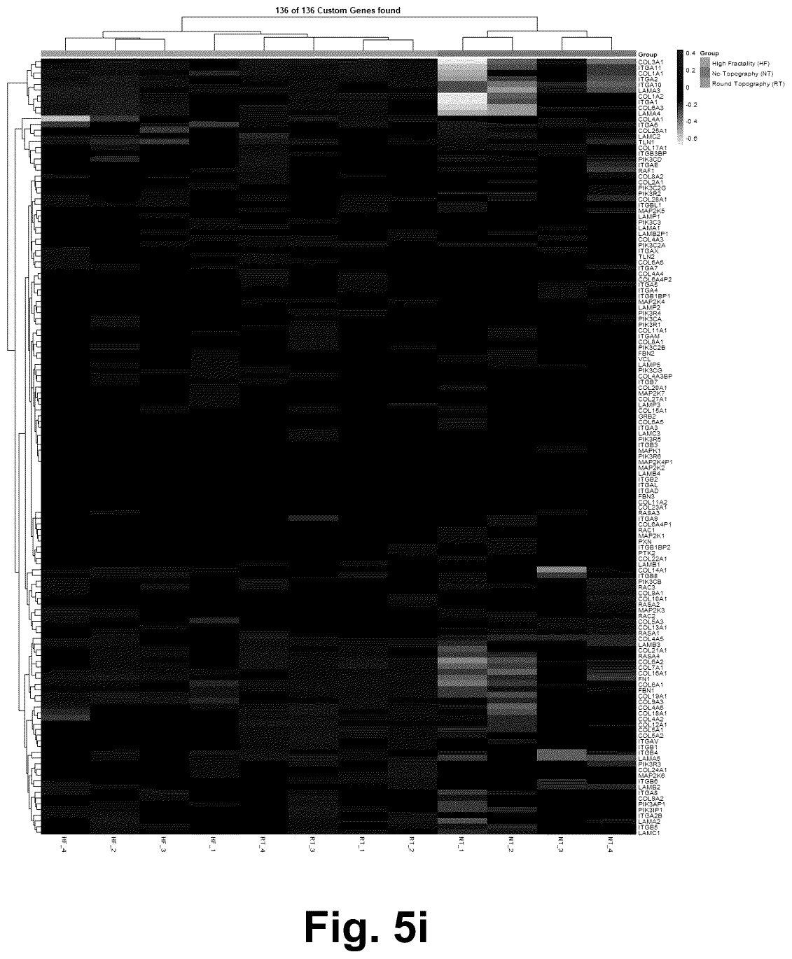

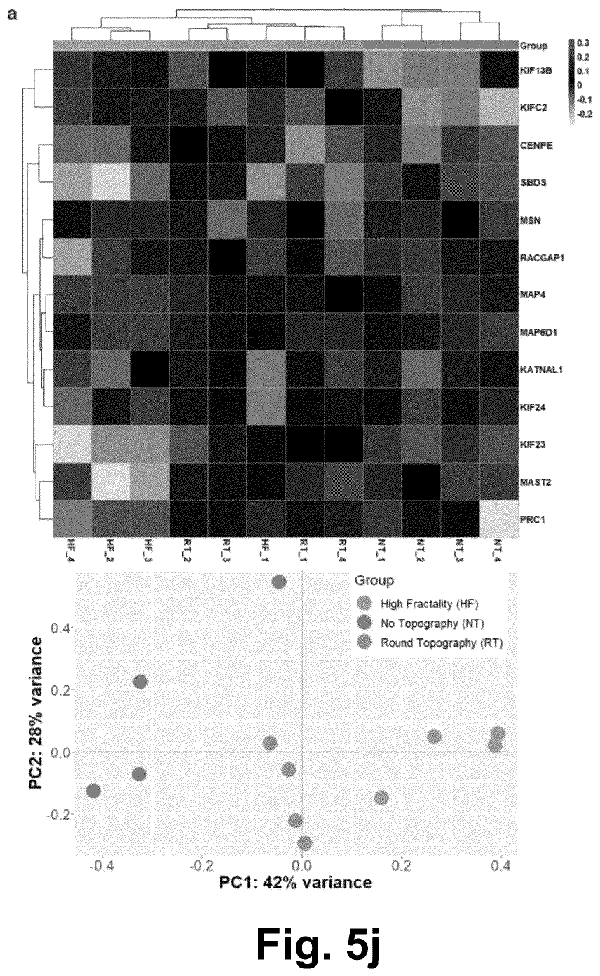

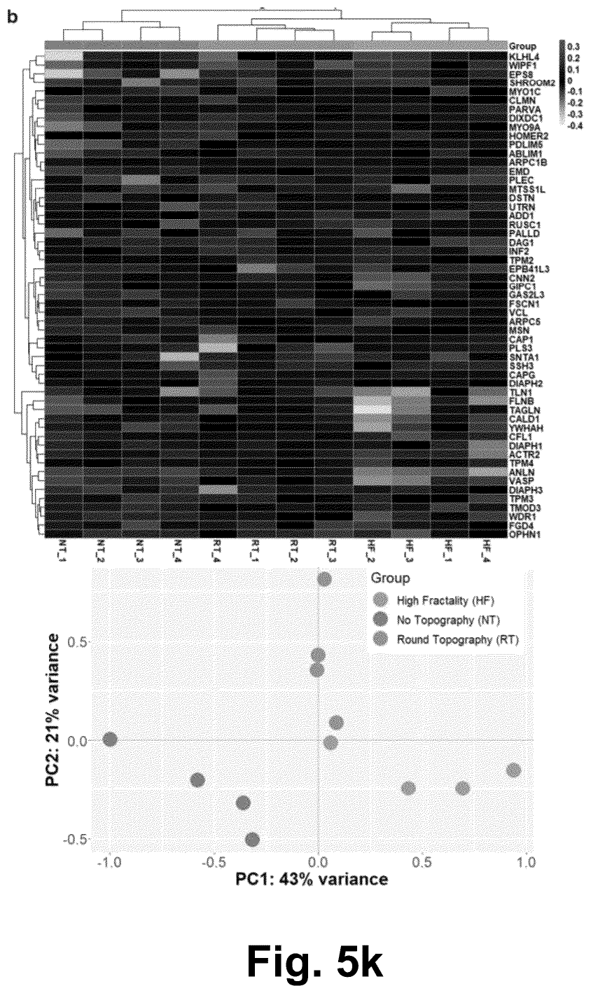

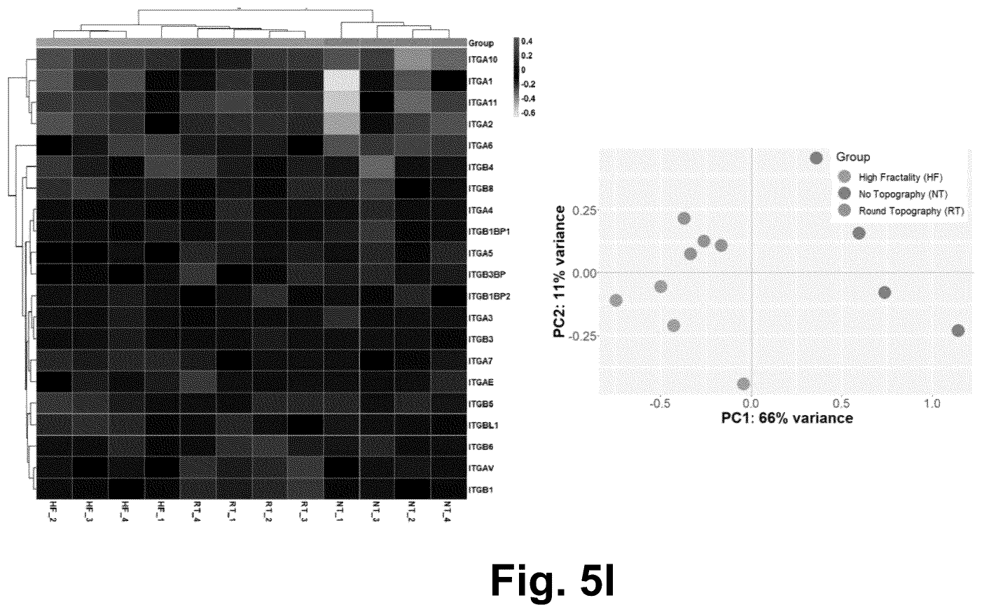

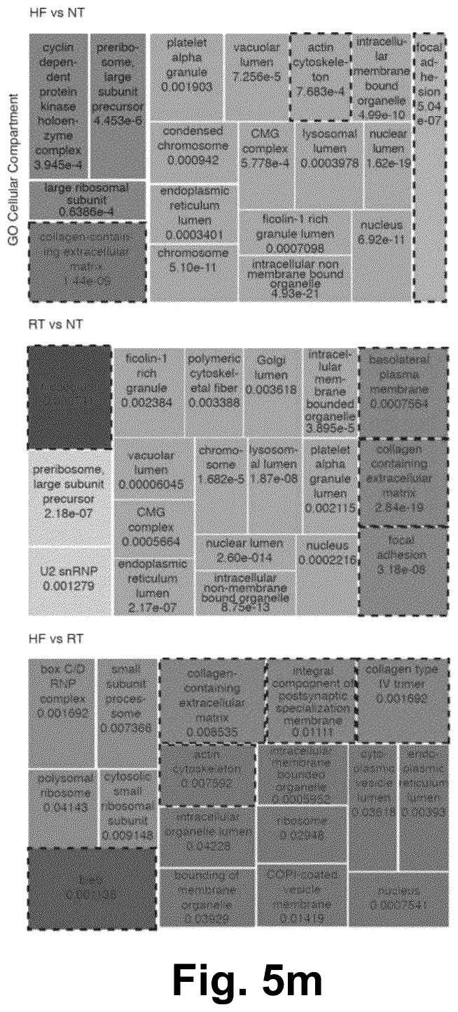

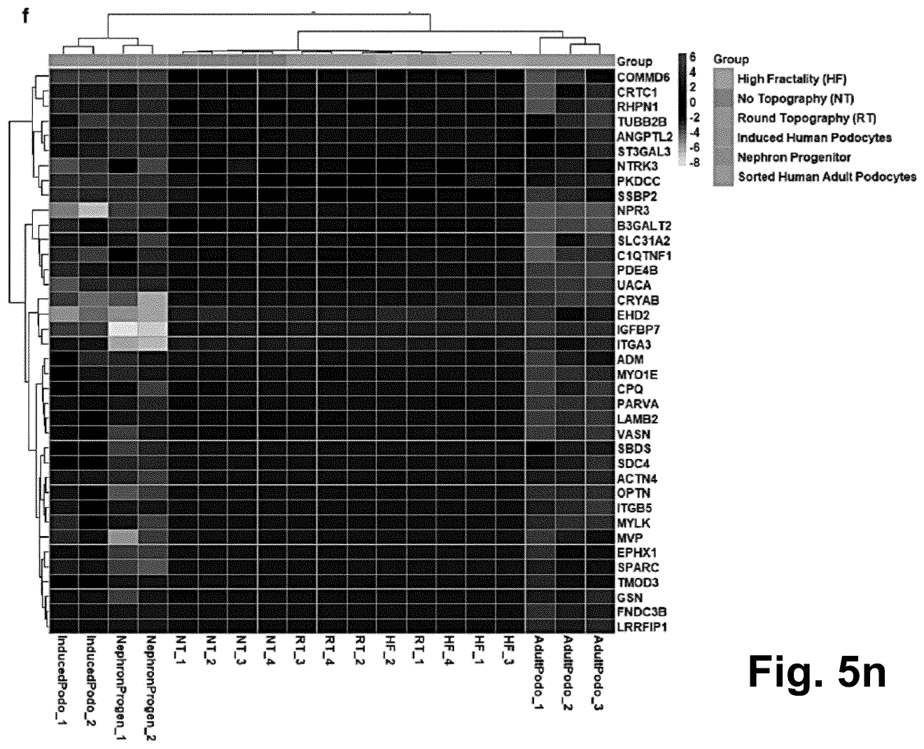

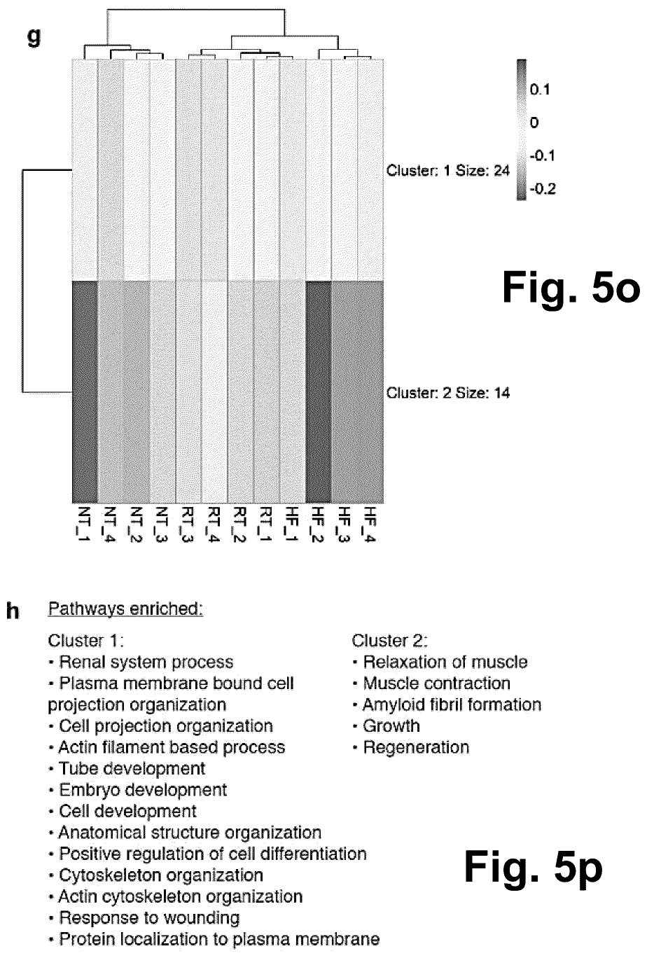

RNA sequencing reveals that fractal topography modulates human primary podocyte response via ECM synthesis, actin-regulation, and synaptic-like branching gene expression mechanisms. Unsupervised principal component analysis (PCA) effectively clustered the three scaffold groups based on their gene expression differences, accounting for 36% (PC1) and 15% (PC2) of variance (FIG. 5a). Using DESeq2, significantly differentially expressed genes (DEG's) were identified between groups: 2169 between HF-NT, 1241 between RT-NT, and 386 between HF-RT, with a false discovery rate less than 5%, supporting a dose-test-like response trend as topographic stimulation is made more fractally complex (FIG. 5b). The PluriTest, which plots expression of pluripotency markers indicative of cell “stem”-ness, further classified the groups to reveal a trend towards maturation with fractal topographic stimulation (FIG. 5c inset).

Next, the full gene expression signatures from each group was assessed, using a comprehensive list of podocyte-relevant genes from Park et al.³², to extract which functional processes and compartments were altered by the topography treatment. Unsupervised k-means clustering determined four clusters for which gene set enrichment analysis identified enhanced signaling pathways (FIG. 5d). Again, in a dose-like response, HF had enrichment in pathways associated with extracellular structure and matrix organization, nephron and epithelial development, motility, and structure formation (FIGS. 5e, 5f(1)-(2) and 5g). Volcano plots of abundant and significant DEG's for each comparison, manually annotated for functional association, also revealed enhancement of ECM- and morphogenesis-associated genes in RT and HF, while cells in the NT group had more proliferative and cell cycle-related pathways (FIGS. 5h(1)-(3)). Importantly, genes additionally upregulated by fractal patterning on top of RT alone included LRFN5, GLI3, VPS8, and MFGE8, which are associated with membrane activity like cell adhesion and glycoprotein-based endocytosis, as well as a developmental activity like membrane outgrowths and limb digit number specification, suggesting that fractality affects fine-tuning of membrane processes. By manual curation, a comprehensive heat map of ECM-associated genes (FIG. 5i), as well as microtubule-binding, actin-binding/stabilizing genes, and integrins (FIGS. 5j-5l), further corroborated clustering and upregulated expression in response to increasing fractal topography. Further, mapping of gene ontology (GO) terms revealed that membrane extremity and ECM binding-associated cellular compartments (e.g., synapses, filopodia, and blebs, cytoskeleton and extracellular matrix proteins) were the locations of differential activity (FIG. 5m).

The gene expression of topographically-stimulated cells was examined relative to adult podocytes and to biochemically-stimulated iPSC-derived podocytes (from GSE116471 by Yoshimura et al.⁷). Biochemical iPSC-podocytes had higher maturation on the PluriTest score, though both biochemical and topographic cells were more mature than nephron progenitor cells (FIG. 5c). Importantly, podocytes grown on fractal scaffolds had a unique signature of gene expression consistent with the adult podocytes for critical genes that were lacking in biochemically stimulated cells (FIG. 5n). Unsupervised k-means clustering and gene set enrichment analysis of this newly identified subset of genes that were uniquely enriched in topography revealed significant upregulation of pathways in podocytes associated with relaxation/contractility processes, fibril and membrane projection formation, growth, and differentiation, as well as cytoskeleton and protein localization and organization (FIGS. 50, 5p, 5f(1)-(2) and 5g).

Taken together, these findings support fundamental differences between groups with different extents of fractal topographic stimulation, and suggest that a main biological response to biomimetic curved shape stimulation was associated with ECM manipulation and structural organization—in the synthesis, organization, and binding to ECM proteins, as well as in the organization of cell cytoskeleton, protein localization, and morphology. These findings are consistent with the fluorescence characterization as well, and phenotypes were found to be mechanistically supported with several proposed causal networks.

Fractal topographic cultures respond to applied stimuli with physiological relevance. It was demonstrated that the utility of the miCRAFT platform as an in vitro model for screening injury in podocytes.

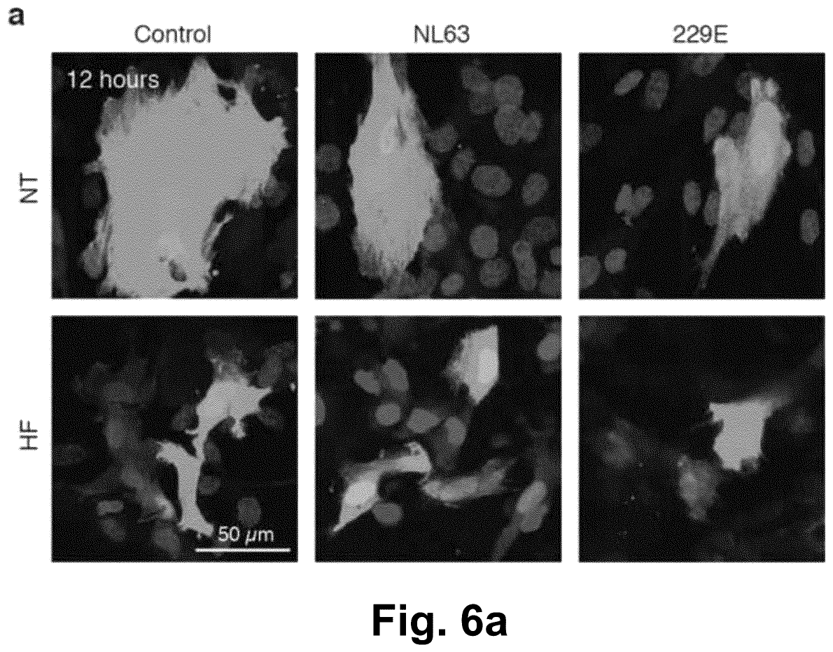

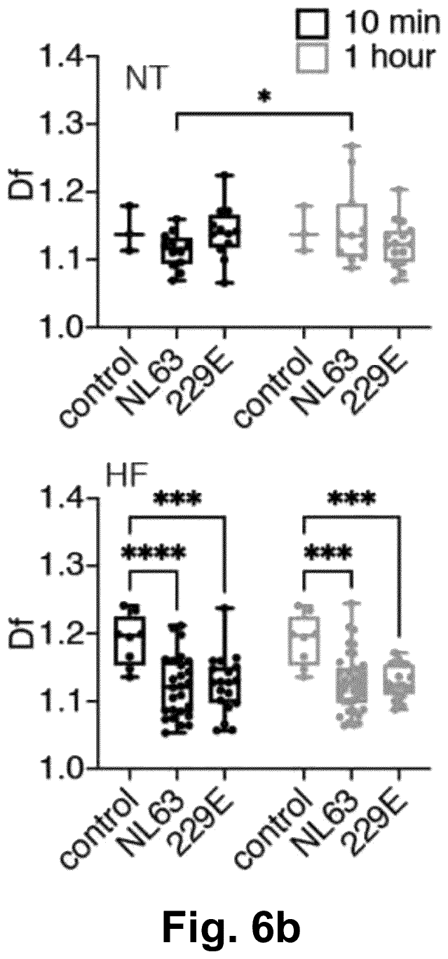

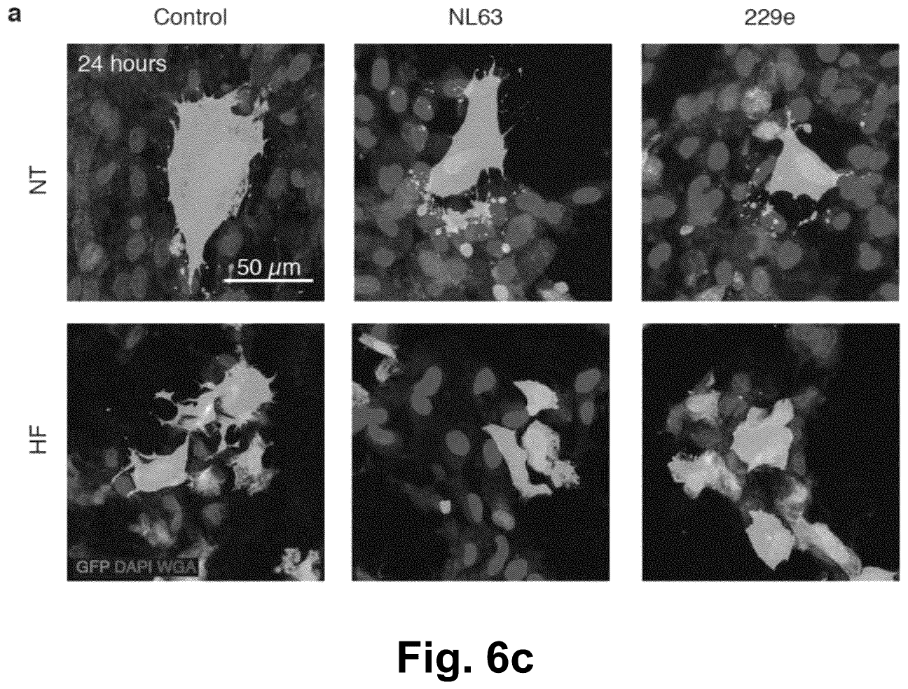

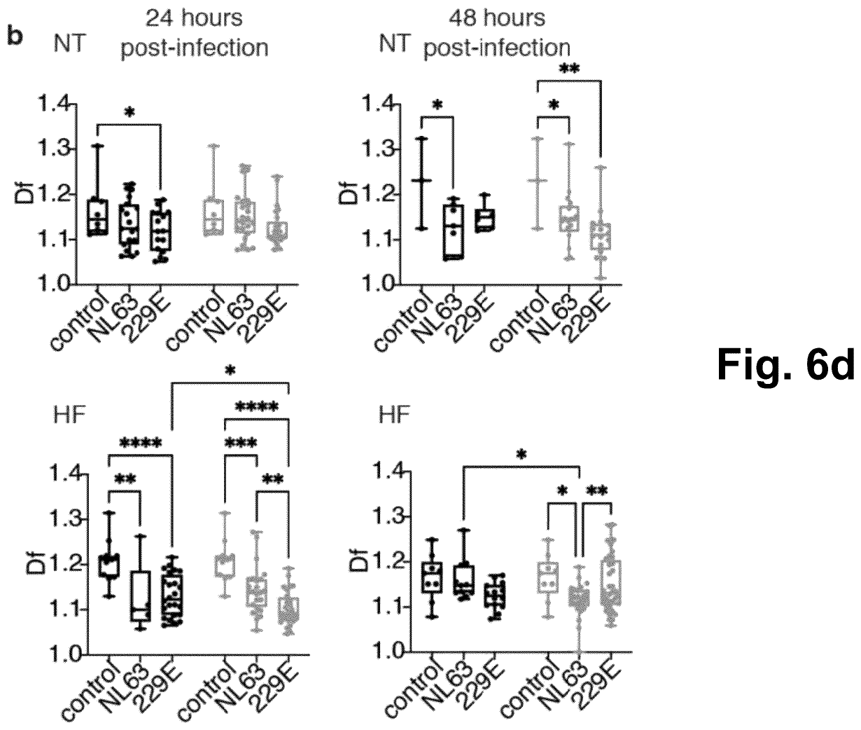

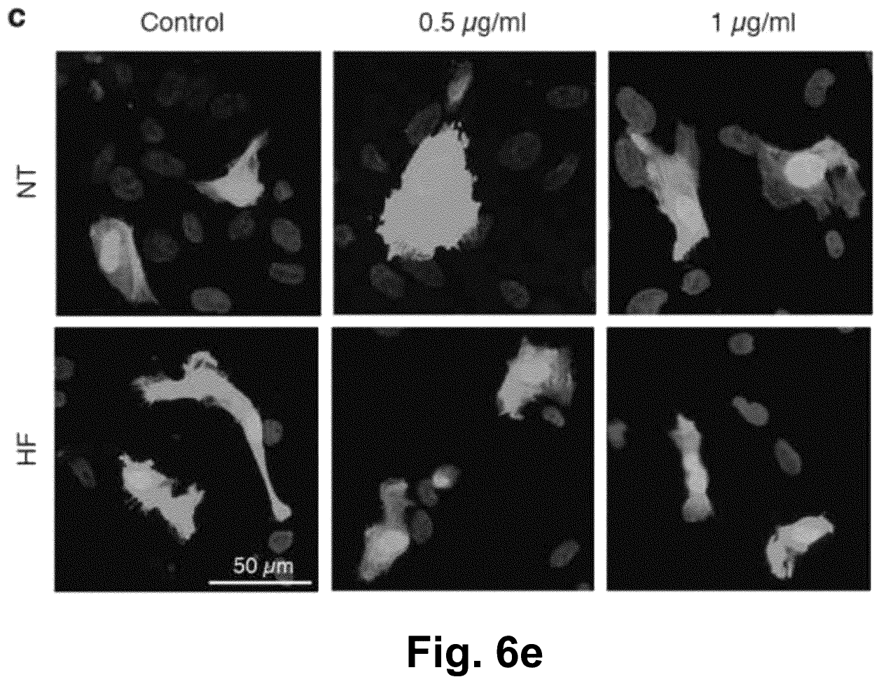

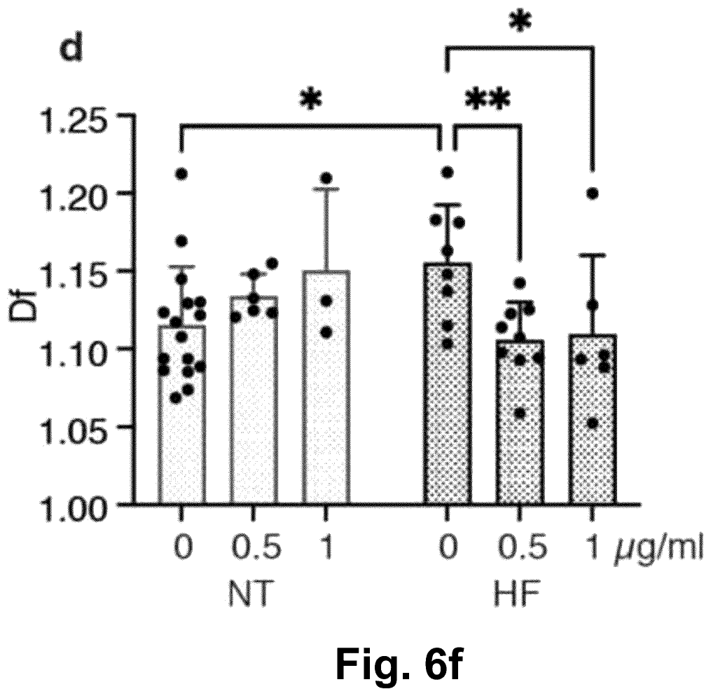

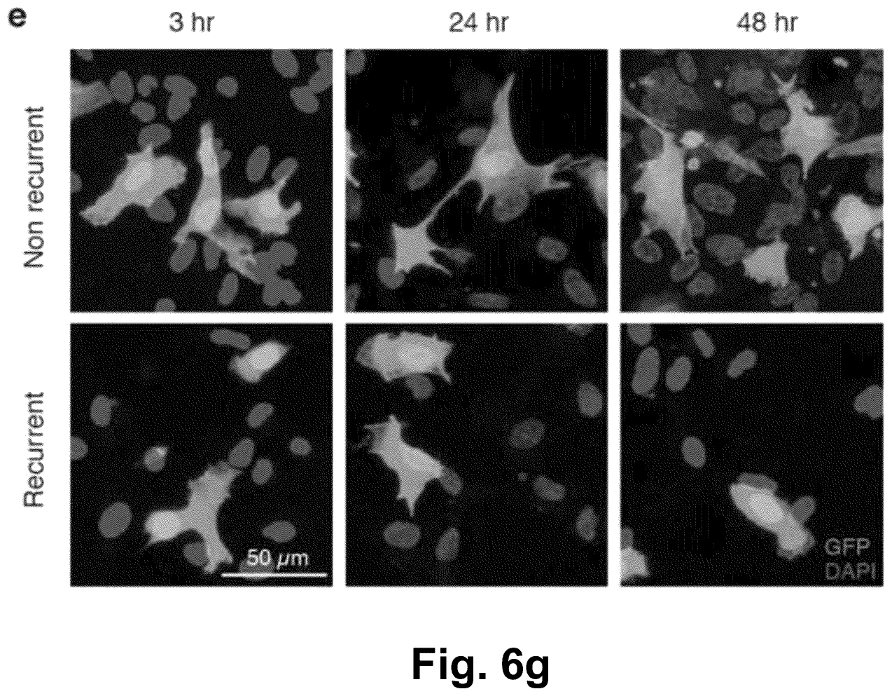

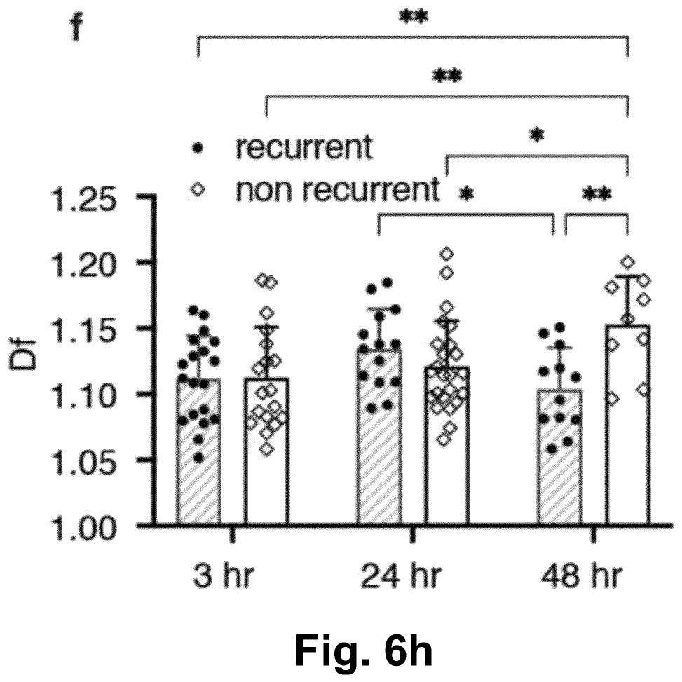

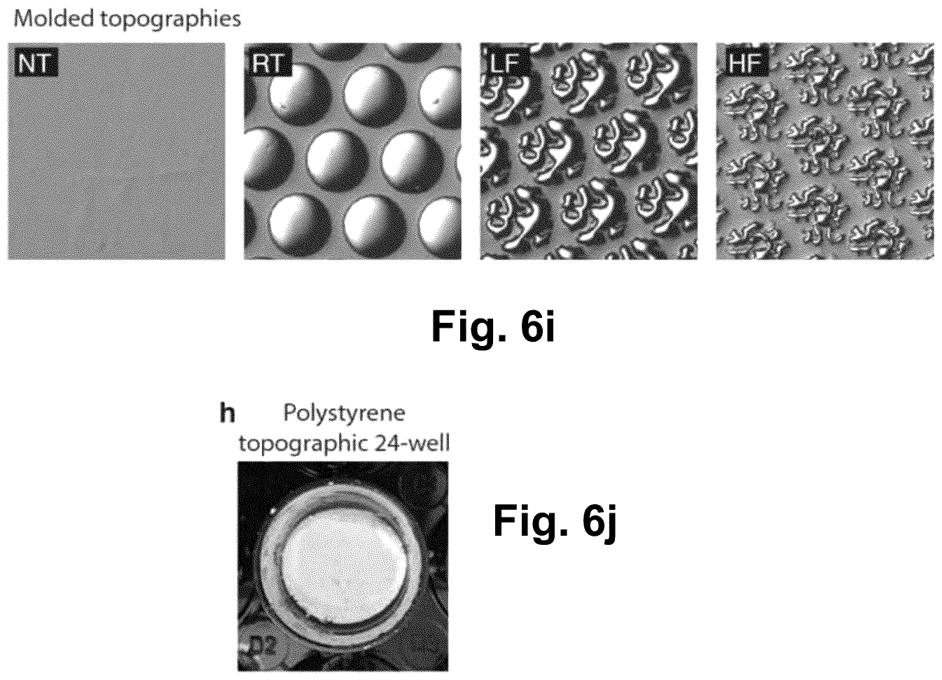

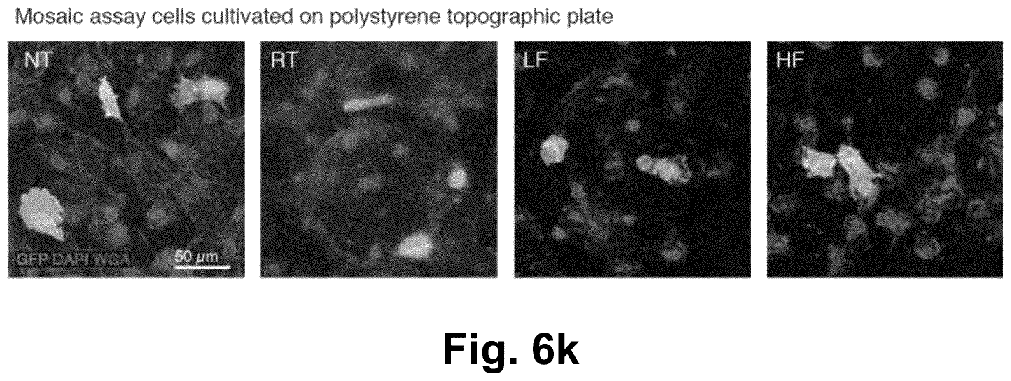

Certain coronaviruses are known to directly infect podocytes³³. SARS-CoV-2 and HCoV-NL63 bind the angiotensin-converting enzyme 2 (ACE2)^{34,35}, and HCoV-229E binds to aminopeptidase N (APN), both expressed in podocytes³⁶. Viral injury was modelled in Mosaic cultures of immortalized human podocytes by incubating the human coronaviruses HCoV-229e and HCoV-NL63 for 10 minutes or 1 hour on HF and NT scaffolds, and monitoring for a reduction in fractal cell morphology typical of foot process effacement over time (FIGS. 1c-1f) (FIG. 6a). Podocytes on the HF scaffold demonstrated a significant and virus-specific reduction in cell fractal dimension and less cell branching as early as 12 hours post-infection that was not detected by NT cells (FIG. 6b). The response on HF scaffold peaked at 24 hours post-infection. Meanwhile, podocytes on NT had a delayed and less significant response that only appeared at 48 hours post-infection (FIGS. 6c and 6d).

Next, in the application of drug testing, Adriamycin (i.e., ADR; doxorubicin) is a chemotherapeutic with known nephrotoxicity often used to induce nephropathy in animal models³¹. A dose test demonstrated a sensitive injury response to 0.5 and 1 μg/mL ADR by reduced fractal dimension of cell morphology only with Mosaic cells cultured on HF scaffolds (FIG. 6e, 6f).

To assess clinical relevance, sera from patients with primary focal segmental glomerulosclerosis (FSGS) were investigated, a glomerular disorder leading to kidney failure for which up to 40% of transplant patients with a kidney transplant may experience recurrence of the disease to their new graft³⁸. Although there is no predictive test for disease recurrence, it is thought that these cases with post-transplant recurrent disease may have a circulating factor or other blood-related anomalies that are nephrotoxic, which are absent in cases with no disease recurrence³⁹. When Mosaic podocytes on HF scaffolds were intubated with serum samples (FIG. 6g) from three different patients from pre-transplant recurrent and non-recurrent FSGS clinical groups each (six patients total) (Table 1), after 48 hours the recurrent serum-treated cells had a significantly reduced fractal morphology compared to both its own previous 24-hour time point and to the non-recurrent patient serum-treated cells (FIG. 6h). Non-recurrent serum cells instead exhibited overall improving morphology after 48 hours in culture characterized by an increased Df of morphology (FIG. 6h); this improvement may be expected when species of serum and cells, in this case both human, match, vs. a mismatch of bovine serum and human cells⁴⁰.

Finally, to demonstrate broad applicability and scalability of the 2½-D fractal cultivation and readout system, the topographic molds were hot-embossed out of polystyrene, a chemically inert tissue-culture plastic that, unlike PDMS, is non-absorbent. The features were clearly imprinted (FIG. 6i) and successfully assembled with a bottomless well plate for cell culture (FIG. 6j), where cells again clustered in island-like patterns on top of the “slice” topography (FIG. 6k), suggesting consistent behavior of polystyrene and PDMS, both of which demonstrate negative surface charge in solution⁴¹ (FIG. 2f). These results pave the way to the scale-up of topographic substrate production, a requirement for translation and broader adoption.

These results support the effectiveness of the fractal platforms, both scaffold, and Mosaic readout, in representing a coordinated response to complex factors that may serve as an exemplar of a scalable cells-as-sensors diagnostic device for detecting kidney injury.

Discussion and Results

Overall, the data showed that biomimetic fractal patterning of curved shape cues was implicated in the structural organization of ECM and hierarchical assembly of cell machinery required for podocyte maturation. The results suggest a possible sequence for how fractal shape cues may drive cell development: (1) fractality increases surface area and local Gaussian curvature patterns charge density gradients in space, which (2) supports and organizes ECM protein adhesion and subsequent enrichment of cell integrin binding into precise geometries, to finally facilitate (3) multiscale assembly, localization, and balancing of cell structures, cumulating in higher-order branching morphology. The first experimental demonstration of fractal patterning contributing functionally to tissue behavior is presented, which may support ongoing theoretical derivations and development of advanced functional materials. It is possible that fractal patterning of stimuli, based on a multiscale feedback process, may encourage more chaotic dynamics in the system. Appropriate fractal stimulation may also become an additional useful tool in other systems, such as hematopoietic stem cells² and neuroepithelial bodies⁴², that are known to preferentially locate on fractally bifurcating microenvironments; or in recellularization of decellularized tissues that result in patterned cell distributions on the stripped-down skeleton⁴³.

Where non-fractal techniques fell short of capturing the full significance of structural complexity, native glomerular histology and podocyte morphology with fractal dimension were successfully quantified to uncover a previously undocumented correlation with the state of tissue health, and harnessed this knowledge to build a physiologically relevant in vitro model of podocytes. The 2½-D approach to biomimetically templating fractal, high surface area, materials presents a controlled alternative to molecular self-assembly techniques for generating fractal materials⁴⁴. While flat regions exist between individual patterns on topographic scaffolds, the overall presentation of nonlinear curvature is more representative of native heterogeneity than perfectly ordered and flat substrates. Further application of the fractal approach with the Mosaic assay demonstrated a solution to in vitro delineation and characterization of complex podocyte interdigitating morphology, thus presenting a reliable, quantitative, and cross-referenced proxy of podocyte function; a standardizable readout that is presently lacking in the field.

While gene expression signatures contribute to cells generating the correct set of building blocks for a given cell identity, the work in identifying ECM manipulation, protein localization, and structure development as maturation pathways specifically enriched in response to topographic stimulation, supports that correct placement of protein building blocks is the other element critical to advancing maturation of cells in vitro, which shape stimulation uniquely encourages. These processes were consistent with the observed cluster formation and morphology elaboration by immunofluorescent imaging, and were harnessed successfully to develop cells-as-sensors applications in injury modeling, drug screening, and disease diagnostics. The miCRAFT system demonstrated capability to sensitively respond to injurious factors in recurrent FSGS patient blood, suggesting that other diagnostic applications should be feasible to develop.

### Second Example Study

In this study, a single Df value was focused on, to balance experimental feasibility of constructing physical scaffolds of clearly delineated patterning categories with the biology of the cells cultured thereon.

Method of Fabricating Cell Culture Apparatus

Glomerular Histology Tracing: Images of kidney histology sections from the literature were manually cropped in Fiji to only show glomeruli. Next, the images were thresholded using the Otsu filter in Fiji to create a binary mask that cover the cellular/protein space, followed by despeckling once to reduce the background noise. The binary mask was then outlined. Lastly, the outlined traces were quantified in Fiji using the FracLac plugin for fracal dimension analysis.

Fractal Dimension Analysis: The fractal dimension analysis was performed using either the box counting algorithm from the Fraclac plugin in ImageJ or the box-counting method through a Matlab code developed by Frederic Moisy.^{[A42]} Fraclac was used to generate single-value Df; the Matlab code was used for multi-fractal analysis. Briefly, outlined traces were input to Fraclac and box counting, on binary images with the background locked to white, was applied for fractal analysis. The method developed by Frederic Moisy involves covering the image with a set of boxes of size R, and counting the number of boxes (N) that are required to cover the entire image. The relationship between N and R can be expressed as:

N=N₀R^{D}^{_{F}}

D_{F}≤D

where D_{F} is the fractal dimension or the Minkowski-Bouligand dimension, and D is the space dimension. By plotting the local exponent, the local fractal properties can be identified within a limited range of box size R using the equation:

$${D}_{F} = - d \frac{ln ( N )}{ln ( R )}$$

Fast Fourier Transform (FFT) Analysis: the FFT analysis was employed to investigate the frequency components present within the images. The FFT function available in ImageJ was used to compute the FFT image of the data. The resulting FFT image was then subjected to surface plotting and profile plotting of the central vertical line.

Fractal Pattern Fabrication: The master molds with patterns were fabricated using the method described above.^{[A14],[A15]} Briefly, the HF design units were as densely packed as possible in AutoCAD to minimize the gaps and arrayed to a square area when imported to a direct laser writer (MicroWriter ML3 Baby). A silicon wafer was first coated with an HMDS adhesion layer by spinning at 3000 rpm for 30 seconds and softbaked at 150° C. for 1 minute. Positive photoresist AZ P4620 was then spin coated at 1500 rpm for 30 seconds, softbaked at 110° C. for 80 seconds, then rehydrated for approximately 30 minutes at room temperature. The design was laser patterned at 770 mJ/cm², developed, and re-flowed to generate curvature by baking at 120° C. on a hot plate for 1-2 minutes.

PDMS Substrate Fabrication: Patterns from master molds fabricated by direct laser writing of AZ P4620 positive photoresist on silicon wafer were inverse molded with PDMS at a 1:10 ratio. These PDMS inverse molds were then baked and fully cured at 120° C. overnight and used as a master mold to make topographic PDMS substrates, used as scaffolds for cell culture. Scaffolds were punched out from the PDMS to create circular inserts that fit in tissue culture well plates.

Substrate Pattern Characterization: Patterned substrates were imaged with a 3D digital microscope (Keyence) with partial coaxial illumination in z-stacks to acquire their surface profiles. The stacked microscope images were then reconstituted into 3D display. Patterned substrates were also imaged under scanning electron microscopy (SEM, Hitachi TM4000 or SU7000) at 5.0 kV voltage acceleration to confirm feature transfer from master molds to scaffolds.

Cultivating the Cells

Cell Culture: The conditionally immortalized E11 murine podocyte cell line was a kind gift from GSK. Murine podocyte E11 cell line (referred to as mouse podocytes) was expanded in rat tail collagen I-coated flasks under a proliferative condition at 33° C. with 10 units/mL interferon gamma supplemented in fresh in RPMI 1640 culture media containing HEPES and GlutaMAX, with 10% FBS, and 1% pen-strep. The Podo/Tert256 human podocyte hTERT immortalized cell line (referred to as human podocytes, Evercyte) was grown in human collagen I-coated flasks at 37° C. in MCDB101 basal medium, supplemented with 20% FBS, 2 mM GlutaMAX, 12 μg/mL Bovine Brain Extract, 10 ng/mL hEGF, 25 ng/mL hydrocortisone, and 100 μg/mL G418. For both mouse and human podocytes, media was changed every 2-3 days.

Coatings: Coatings were applied to substrates prior to cell culture and seeding. Matrigel (Corning, #354234) was diluted 1:60 in DMEM/F12 basal media and incubated for 2 hours at 37° C. Rat tail collagen I (Corning, #354236) was diluted in PBS at 0.1 mg/ml and incubated for 1 hour at 37° C. Human collagen I (Sigma, #C7774) was diluted in PBS at 6 μg/cm² and incubated for 1 hour at 37° C. Porcine kidney ECM (Xylyx Bio, NativeCoat Kidney ECM surface coating kit #MTSKY201) was diluted in kit buffers according to manufacturer's instructions, at 0.08 mg/mL, and incubated for 2 hours at 37° C. Appropriate coating is critical for successful cell culture, and natural ECMs such as porcine kidney may exhibit batch-to-batch variability that will require optimization of the coating procedure and cell density. Most consistent and reproducible results are obtained if Matrigel is used for coating.

Substrate Preparation for Cell Seeding: PDMS substrates were sterilized by autoclave and inserted in well plates to allow for standard cell culture techniques to be applied for cell seeding and cultivation on the scaffolds. In addition to scaffolds, a blunt tweezer was autoclaved at the same time and then used to insert the scaffolds into the wells of a well plate. Each scaffold was pushed to the bottom of the well using the tweezer to ensure all edges of the scaffold were flat in the well and to eliminate the gap space between the scaffold and the well. By doing so, fewer cells would be lost in the gap space and not settle on the substrate surface when the cells were seeded. After inserting the scaffolds, PBS was added to the wells to wet the scaffold surface and the well plate was incubated at 37° C. for at least 30 minutes. Coating materials were then added on top of each scaffold and incubated at 37° C.

Cell Seeding: Seeding densities were tailored for each cell source to adjust for different growth rates and spreading dynamics. Mouse podocytes were seeded at an initial density of 50,000 cells/cm² and allowed to attach and reach ˜80% confluence under the proliferative condition before thermoswitching to the differentiation condition at 38° C. in DMEM-F12 media with 10% FBS and 1% pen-strep (without interferon gamma) and culturing for an additional 14 days prior to end point analyses. Human podocytes were seeded at an initial density of 50,000 or 100,000 cells/cm², and grown until ˜80% to full confluence as the study endpoint (˜5 days). Various seeding densities (50,000, 100,000, and 240,000 cells/cm²) were tested to find the optimal seeding density for a robust protocol of obtaining a confluent sheet of human podocytes on the fractal topographic scaffolds. The recommended protocol is 50,000 cells/cm² for a culture period of 5-6 days or 100,000 cells/cm² for a culture period of 3-4 days on Matrigel coated scaffolds. Depending on the scaffold materials, further optimization of seeding density and culture period may be required. To seed the cells, harvested cells from the expansion cultures were resuspended at a concentration corresponding to the target seeding density. After that, the cell suspension was pipetted up and down a few times to mix evenly and then added to each well. Finally, the well plate was left in the incubator (37° C. for human podocytes, 33° C. for mouse podocytes) for the cells to attach. Media change was performed the next day or in two days. The same culture media was used for human podocytes whereas media was switched to differentiation media for mouse podocytes.

Imaging and Analysis

Glycocalyx Imaging: For glycocalyx imaging, samples were fixed with 4% PFA, 1% glutaraldehyde, and 1% Alcian Blue overnight. They were then post-fixed with 0.5% osmium, serially dehydrated in ethanol, dried at the critical point (CO₂), and gold-coated for 2 minutes. Samples were imaged using a Hitachi SU-8230 scanning electron microscope, using 2.0 kV voltage acceleration.

Immunofluorescent Staining: At the endpoint of each experiment, cells were fixed with 2% PFA for 20 minutes at room temperature, and washed three times with PBS. Fixed cells were blocked and permeated in 5% normal goat serum (NGS) with 0.1% triton X-100 for 1 hour at room temperature under gentle shaking. The blocking solution was diluted to 2% NGS using PBS to prepare primary and secondary staining solutions. The dilution factors of antibodies and stains are listed below. Without rinsing, cells were left with primary solution overnight at 4° C. After that, cells were washed with PBS for three times. Secondary staining solution containing counterstain was then applied for 1 hour at room temperature under gentle shaking. After secondary staining, scaffolds were washed three times with PBS and carefully taken out of wells. Excess liquid was removed from the bottom of each scaffold with paper towel. Three scaffolds were placed on each microscope slide. Next, mounting media containing DAPI (Vector Laboratories, Vectashield #H-1200) was applied at 6 μL per scaffold. Lastly, a cover slip was placed on top of each scaffold for even spreading of the mounting medium. Care should be taken when handling a scaffold to make sure not to disturb the delicate sheet of cells on top. A blunt tweezer should be used to carefully take a scaffold out of the well, minimize any contact with the cell side of the scaffold, and avoid twisting or bending the scaffold during handling. Over the course of the staining procedure, scaffolds should be rinsed gently and solutions gently added from the side as opposed to on top.

The following antibodies and stains were used:
Primary antibodies: 1/200 dilution of Rabbit anti-podocin (Abcam, #ab50339)
Secondary antibody: 1/500 dilution of Goat anti-rabbit FITC (LifeTech, #A16105)
Counter stain: 1/500 dilution of Rhodamine labeled Wheat Germ Agglutinin (Vector Laboratories, #RL-1022)

Immunofluorescent Imaging and Analysis: Samples were imaged via confocal microscopy using a Leica Lightsheet Confocal microscope. Z-stacked images were taken to capture cell morphology on topographic substrates. Maximum intensity projection images were acquired from the z-stacks for image analysis. To quantify fluorescence signal, the channel of interest was thresholded in Fiji and raw integrated density was measured. The quantified signal was normalized to cell count in each image frame. The same settings were applied to all the samples during image acquisition, processing and analysis.

Mosaic Assay: Mosaic Podo/Tert256 human podocytes (a mixture of GFP-labelled cells and non-fluorescent cells at a ratio of 1:11) were cultured and seeded in the same way as regular human podocytes as described above. At the study endpoint, cells were fixed with PFA, stained with rhodamine-wheat germ agglutinin (WGA), and mounted with DAPI-containing mounting media as described above. Samples were imaged via confocal microscopy using a Zeiss LSM 880 Super Resolution Confocal microscope to allow sufficient resolution to visualize interdigitated morphology. Z-stacked images spanning the entire section with visible signals were taken for each sample. The images were first processed using Zeiss's ZenDesk software to increase the contrast of GFP-positive cells' morphology as follows: the FITC channel was adjusted by setting gamma to 2.5 and adjusting the white point to 30. The DAPI channel's white point was adjusted to 80. In Photoshop or Fiji, the magic wand tool was used to select single fluorescent cells, create binary masks, and export binary images of single cells. Single cells were verified by overlaying nuclei with the cell images to ensure each cell only contained one nucleus. Binary images were then outlined in Fiji, and fractal analysis was performed using the FracLac plugin where the standard box-counting algorithm was used, with the background locked to white.

EV Isolation: At the study endpoint, cells were rinsed twice with PBS and incubated with serum-free culture media for 48 hours. 8 wells of cell media for each topographic platform were pooled together and collected into a 15 mL centrifuge tube. Samples were spun at 3000 g for 10 minutes at 20° C. to remove cell debris. EVs were then isolated by the miRCURY Exosome Isolation Kit (Qiagen, #76743) based on the manufacturer's protocol. Precipitation buffer was added to the samples at a 4:10 ratio, then rotated overnight in the cold room at 4° C. for EV precipitation. Precipitated EVs were spun at 3200 g for 30 minutes at 20° C. After removing supernatant, the pellet was gently washed with PBS. Isolated EVs were resuspended in 100 μL resuspension buffer for downstream analysis.

Western Blotting: Mouse podocytes were differentiated in 24-well plates, with 6 wells per plate dedicated to each of the 4 surface topographies (NT, RT, LF, HF). On day 14 of culture, cells were washed with ice-cold PBS. 150 μl of RIPA buffer supplemented with a protease inhibitor tablet (Thermo Scientific) was added to a single well for podocyte lysis, pipetting up and down several times while scraping the surface of the well with the pipette tip to release cells. The full volume of RIPA-lysate mixture was then transferred sequentially to the next 5 wells of like-topography one at a time, repeating the process of pipetting and scraping each time. After all 6 wells of a single topography were lysed, the RIPA-lysate mixture was transferred to a microcentrifuge tube and lysis was repeated for the remaining 3 topographies.

EVs were isolated from conditioned media of day 14 cultures of mouse podocytes grown on NT and HF topographies as previously described. EV pellets were lysed in 50 μl RIPA buffer supplemented with protease inhibitor. Cell and EV lysates were centrifuged at 10,000 g and 4° C. for 15 minutes. Supernatants were transferred to fresh tubes and pellets were discarded.

The Pierce BCA Protein Assay Kit (Thermo-Fisher Scientific) was used to analyze protein concentration in lysates. Initial western blots were performed to determine the linear regime for sample loading and target detection. For podocyte cell lysate western blots, samples were loaded at 7 μg total protein adjusted to a volume of 35 μl per well of a 15-well gel; EV blots were loaded at 10 μg total protein per adjusted to a volume of 40 μl per well of a 10-well gel.

Appropriate sample volumes were diluted with 4× Sample Loading Buffer (LI-COR), 10× BOLT Sample Reducing Agent (Thermo Fisher Scientific), and DI water, then incubated at 70° C. for 10 minutes. Samples were loaded into Bolt 4-12% Bis-Tris Gels (Invitrogen). Electrophoresis was perfomed in Bolt MOPS SDS Running Buffer (Invitrogen) at 200V for 35 minutes or until protein front reached the bottom of gels. Completed gels were rinsed in DI water then transferred to Immobilon-FL PVDF membranes (Millipore Sigma) at 20-25V for 7 minutes using the iBlot Gel Transfer Device and transfer stacks (Invitrogen).

REVERT Total Protein Stain (LI-COR) was applied to PVDF membranes according to the manufacturers protocol before imaging on the LI-COR Odyssey Fc Imager (LI-COR). Blocking was then performed by incubating membranes in skim milk buffer for 1 hour. Membranes were washed and stained overnight at 4° C. with anti-PODXL (Invitrogen, PA5-28116, 1:1000 concentration), anti-CD63 (Abcam, ab217345, 1:1000 concentration), or anti-ALIX (Thermo-Fisher Scientific, MA5-32773, 1:1000 concentration). Membranes were then washed, appropriate IRDye800CW secondary antibodies were added (LI-COR, 1:10,000 concentration) for 2 hours, washing was repeated, and images were captured with a 10 minute exposure. PODXL blot was stripped using a mild stripping buffer consisting of 1.5% w/v glycine, 0.1% w/v SDS, and 1% v/v tween 20, titrated to pH 2.2 in deionized water. Blot was incubated in stripping buffer twice for 5 min, washed four times, then blocked as previously described. PODXL blot was then washed and stained as before using anti-GAPDH (Invitrogen, PA1-987, 1:1000 concentration) and IRDye800CW secondary antibodies prior to imaging.

Transmission Electron Microscopy of EVs: Plasma treatment was used to pre-treat electron microscopy grids (Electron Microscopy Sciences). Grids were incubated with 5 μl of 100× EV concentrate for 1 minute before wicking with a wet filter paper. To wash grids, 5 μl of DI water was applied then wicked away. 5 μl of 2% uranyl acetate was then added to grids for 30 seconds as a negative stain. Grids were once again wicked and imaging was subsequently performed on a Talos L120C TEM (Thermo-Fisher Scientific) at 22,000×, 45,000×, and 92,000×.

YAP Inhibition Study: HF and NT scaffolds were prepared as described above. Mosaic human podocytes were seeded on top of the scaffolds at a concentration of 50,000 cells/cm² (100,000 cells per scaffold). Verteporfin (1 or 2 g/ml, Tocris) was added to the culture media 24 hours post seeding. Samples were fixed on day 5. Media was collected and lactate dehydrogenase assay (LDH) assay (Cayman) was performed according to manufacturer's protocol to validate cell viability. Mosaic assay imaging and analysis were performed as described above.

Fractal Topographic Well Plate Fabrication: To produce 24-well plates with a high density fractal pattern, polystyrene sheets were hot embossed against a master mold designed and laser patterned with high density fractal units. The polystyrene base was then cut to the size of a 24-well plate bottom and bonded to the bottomless well plate using PDMS as a glue.

RNA Isolation: RNA extraction and isolation of both mouse podocytes was performed using the PicoPure RNA isolation Kit (Thermo Fisher Scientific, #KIT0204) according to manufacturer's protocol (except 100 μL instead of 50 μL of lysis buffer was applied to extract RNA), including DNase treatment with RNase-Free DNase Set (Qiagen, #79254). At the final step, 12 μL of elution buffer was used. The concentration and quality of isolated RNA was measured using a NanoDrop spectrophotometer. Isolated RNA was stored at −80° C.

Quantitative polymerase chain reaction: Isolated RNA was reverse transcribed into cDNA using the SuperScript™ II RT kit (Invitrogen, #18064-014) according to the manufacturer's protocol. Briefly, random hexamer primer (ThermoFisher, #S0142) was mixed with total RNA (300 ng), dNTP mix (Invitrogen, #18427-013) and water according to the specified quantities. The mixture was heated to 65° C. for 5 min then chilled on ice. Each of the remaining contents of the SuperScript™ II RT kit were then added and incubated as specified in the protocol, with the addition of 1 μL of RNaseOUT™ (Invitrogen, 10777-019) to each preparation. The RT mixture was incubated at 25° C. for 10 min, 42° C. for 50 min, and 70° C. for 15 min.

Each final 20 μL cDNA prep was diluted 1:3 in RNase-free sterile water. qPCR was carried out following the PowerTrack™ SYBR™ Green Master Mix (ThermoFisher, #A46109) kit protocol. A 10 μL reaction volume was used for each PCR well in a 384 well plate, adding diluted cDNA, yellow sample buffer, master mix, and water as specified in the kit protocol. A list of forward- and reverse-primers used is listed in Table 2 below. For each sample, a no-template control well (NTC) without primers was included on the PCR plate as a negative control to ensure no amplification occurred due to sample contamination with genomic DNA/primer dimer formation.

qPCR was performed on a CFX384 Touch Real-Time PCR Detection System following the standard 40-cycle protocol outlined by the PowerTrack™ SYBR™ Green Master Mix kit. For comparison of gene expression, analysis was performed using the ΔΔCₜ protocol, with GAPDH as the reference gene and averaged ΔCₜ values for NT as the control condition from which to determine ΔΔCₜ differences and fold changes for NT and HF data points.

TABLE 2
List of primers used for qPCR

| | |
|---|---|
| GAPDH For | GGGAAGGTGAAGGTCGGAGTC |
| GAPDH Rev | TGGAATTTGCCATGGGTGGAA |
| Nphs1 For | GTGCCCTGAAGGACCCTACT |
| Nphs1 Rev | CCTGTGGATCCCTTTGACAT |

KeyGenes Analysis: In order to quantify the tissue type and the developmental stage of the differentiated podocytes at the different topographical states, the identity score (range 0-1) from the KeyGenes tool was used.^{[A43]} The identity score predicts the “identity” of a test sample to a known feature (tissue or age) by comparing gene expression profiles of classifier genes used in KeyGenes. A higher predicted identity score implicates a greater identity to a specific tissue or differentiation age. The identity score for the topographic replicates against kidney tissue, and adult stage of development is plotted as barplots (mean±SD) using GraphPad Prism. Ordinary one-way ANOVA was used to compare the mean across the different topographic groups.

Heatmaps and Clustering: rlog normalized counts of RNA sequencing data for NT, RT, and HF samples were produced using the DESeq2 package on the Galaxy web platform public server.^{[A44],[A45]} Genes of interest from normalized count files were filtered and plotted using the heatmap2 function on Galaxy^{[A45]} as well as the pheatmap R package (version 1.0.12, https://CRAN.R-project.org/package=pheatmap), employing bidirectional complete Euclidian clustering.

Gene Ontology Analyses: For both of the pairwise comparison sets of HF vs. NT and RT vs. NT, a list of significantly differentially expressed genes (FDR<0.05) and their respective log₂(Fold Change) values were uploaded to the PANTHER knowledgebase for gene ontology (GO) analysis.^{[A46]} The PANTHER statistical enrichment test was used to generate lists of significantly upregulated and downregulated GO terms from the biological process, cellular component, and molecular function databases using a threshold of FDR<0.05.^{[A7]}

YAP1 Interactome: The YAP1 interactome was generated using the GeneMANIA prediction server plugin in Cytoscape.^{[A33],[A34]} Differentially expressed nodes from the interactome were highlighted and plotted on a separate heatmap as described earlier in the Heatmaps and Clustering section.

Statistical Analysis: PRISM was used for statistical analysis by t-test (two-tailed), one-way ANOVA, or two-way ANOVA, with 0.05 considered as significant, and multiple comparisons performed using Tukey's (one-way ANOVA) or Sidak's test (two-way ANOVA). Normality of distribution and equality of variance were tested and verified.

Discussion and Results

Fractal topography was found to increase expression of slit diaphragm markers. To investigate the effect of fractal topography on podocyte culture in vitro, topographical cell culture substrates was used with biomimetic fractal patterns derived from the native podocyte microenvironment observed in histology slices from healthy and pathological samples.^{[A14],[A15]} The design unit of the biomimetic fractal pattern from a healthy histology image was confirmed to be fractal by box counting fractal analysis (FIG. 7f) and packed across a 2D surface to form a patterned area of desirable size. These fractal patterns were then transferred to a master mould via a laser direct writer, which was then used to fabricate fractal topographical substrates made of polydimethylsiloxane (PDMS). To incorporate the patterned substrates into the standard 2D cell culture workflow, they were punched into the size of a well and inserted them into a standard tissue culture well plate (FIG. 7j).

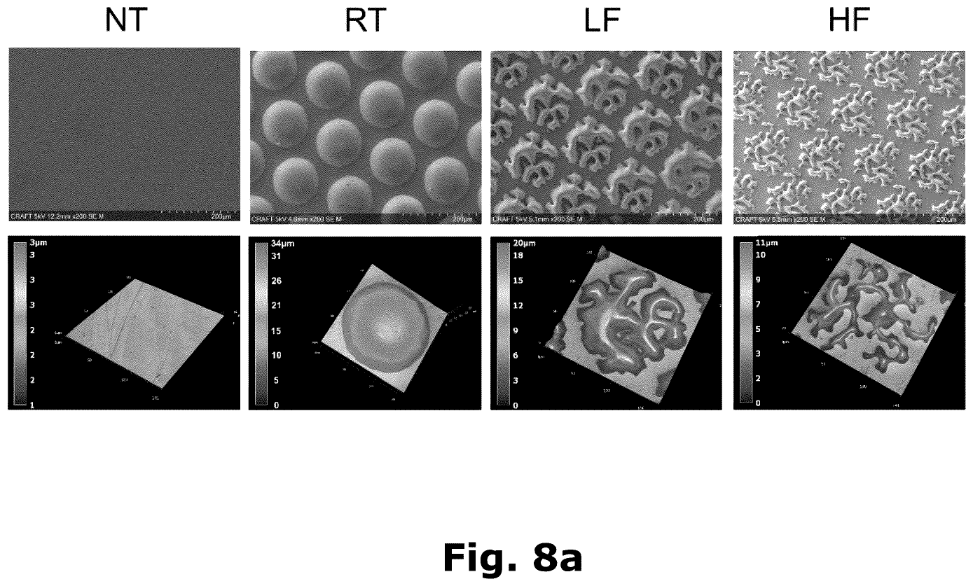

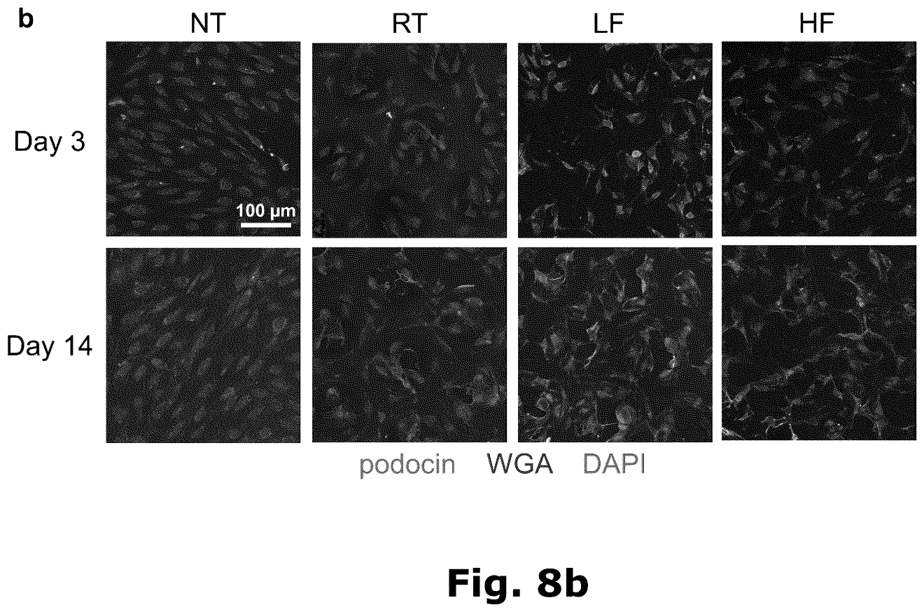

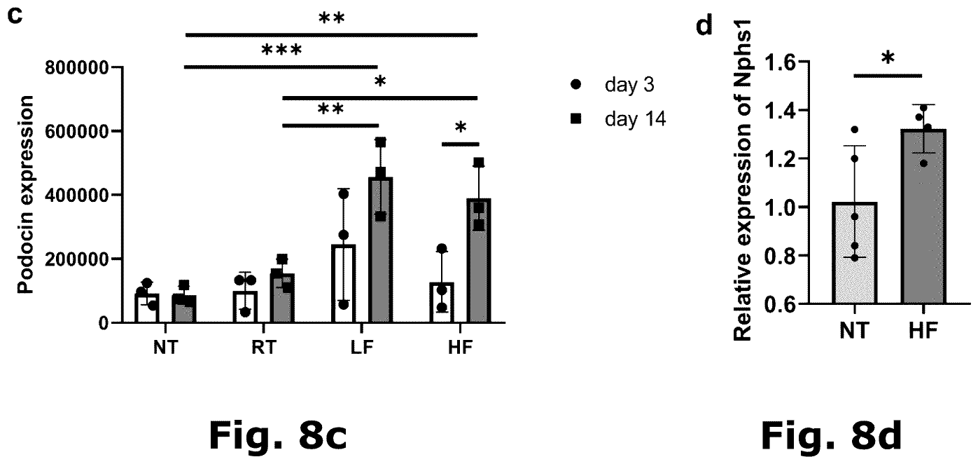

The biomimetic fractal topography corresponding to a healthy state was termed high-fractal (HF) topography and each HF design unit was considered a glomerulus slice with a diameter of approximately 150 μm. As the biomimetic fractal topography derived from a pathological histology image had a lower Df, it was termed low-fractal (LF) topography. To dissect the effect of fractality on podocytes, a round topography (RT) made of an array of microhemispheres each with the diameter of a glomerulus slice was created as a non-fractal control, in addition to the simple flat substrates with no topography (NT).^{[A14],[A15]} The surface features of the substrates from these different groups can be clearly visualized under SEM and 3D digital microscopy (FIG. 8a).

A conditionally immortalized mouse podocyte cell line, E11, is commonly used for studying podocyte biology since human podocyte cell source is scarce and the conditional immortalization preserves podocytes' terminal differentiation state while allowing for cell expansion. The gene expression of a slit diaphragm marker, Nphs1, from E11 podocytes grown on NT and HF substrates was first assessed by quantitative polymerase chain reaction (qPCR) and found a significantly enhanced expression on HF compared to the NT control (FIG. 8d). In FIG. 8d, data is normalized to NT, student's t-test is used for statistical testing, and data are shown as average±SD. p≤0.05 is considered significant.

Next, the expression of another slit diaphragm marker, podocin, from podocytes cultured on flat (NT) and topographical (RT, LF, HF) substrates was evaluated over time. E11 podocytes has a thermoswitching mechanism that could deactivate the forced cell cycle processes and switch from a proliferation to differentiation mode. A cultivation period of at least 14 days is considered enough for differentiation and commonly used. As such, day 3 and day 14 were selected as the early (immature) and late (mature) endpoints, respectively. For true interdigitation, cell-cell contact is needed. Thus, structures can only be reliably observed once the cells are nearing confluence and beyond. Cells cultivated on NT substrates tend to proliferate more, thus achieving conditions for formation of inter-digitations faster (FIG. 8b). Although in early cultures (Day 3), HF substrates did not appear to have substantial podocin presence (FIGS. 8b-c), as the culture time increased fractal substrates exhibited a clear improvement of podocin presence over flat substrates, whereas cells on flat substrates exhibited levels as low as on Day 3 even when cell culture was taken to Day 14 (FIGS. 8b-c). For FIG. 8c, two-way ANOVA was used with Tukey's multiple comparisons test between groups within day 3 and day 14, respectively, and with Sidak's multiple comparisons test between day 3 and day 14 for each group.

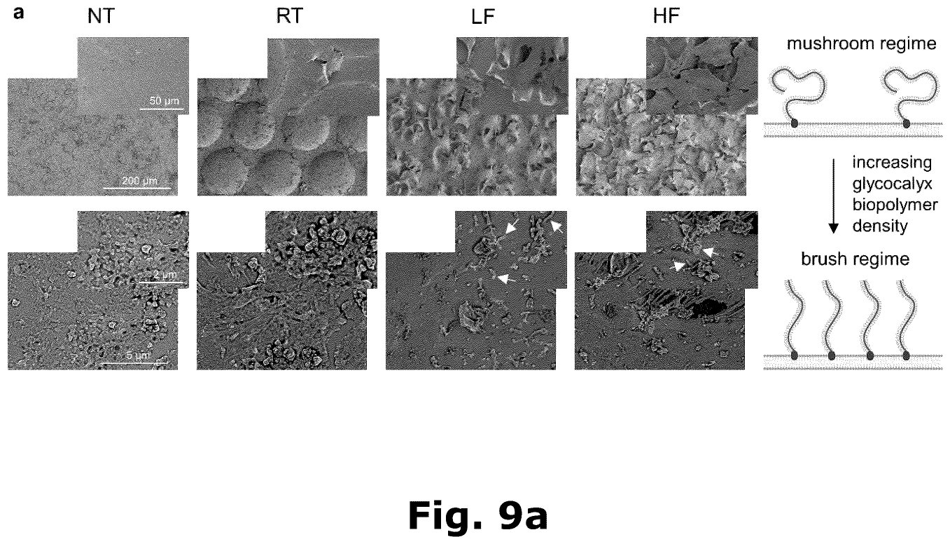

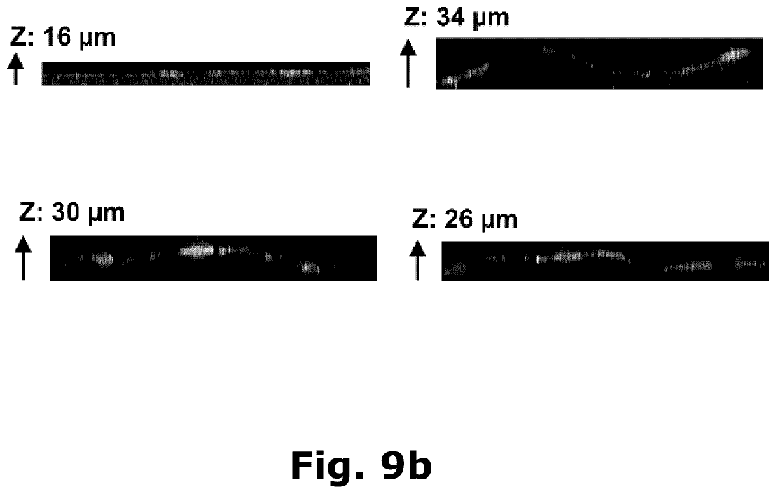

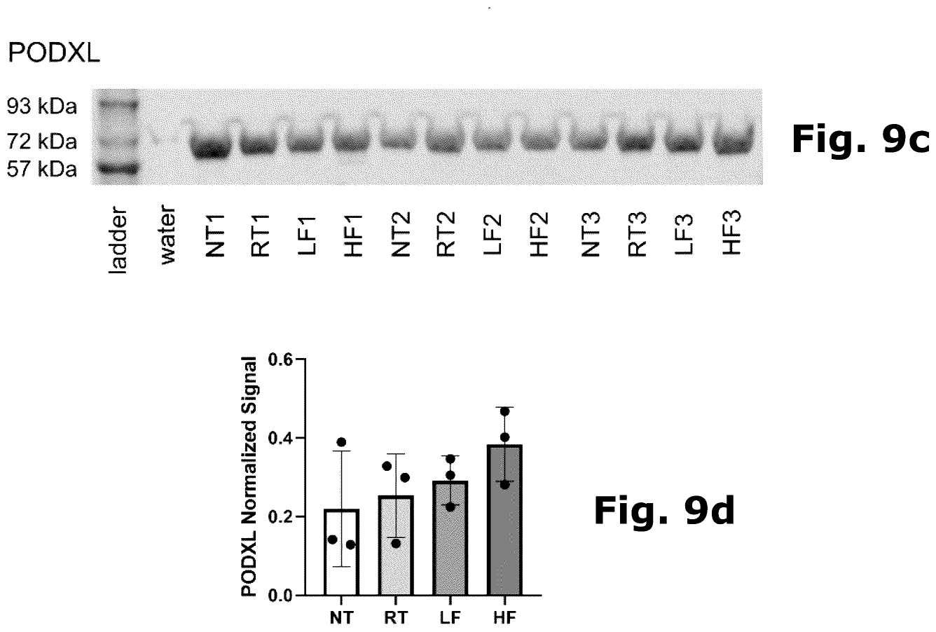

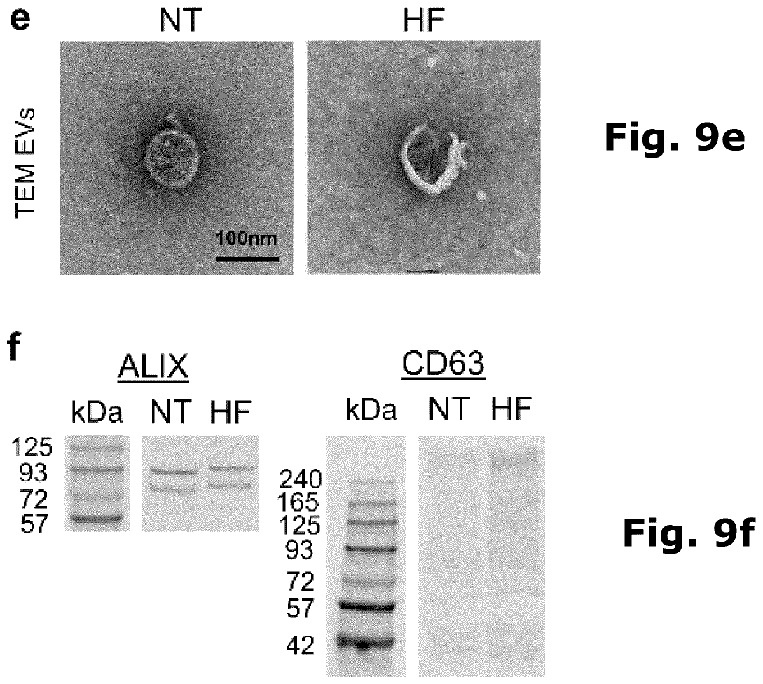

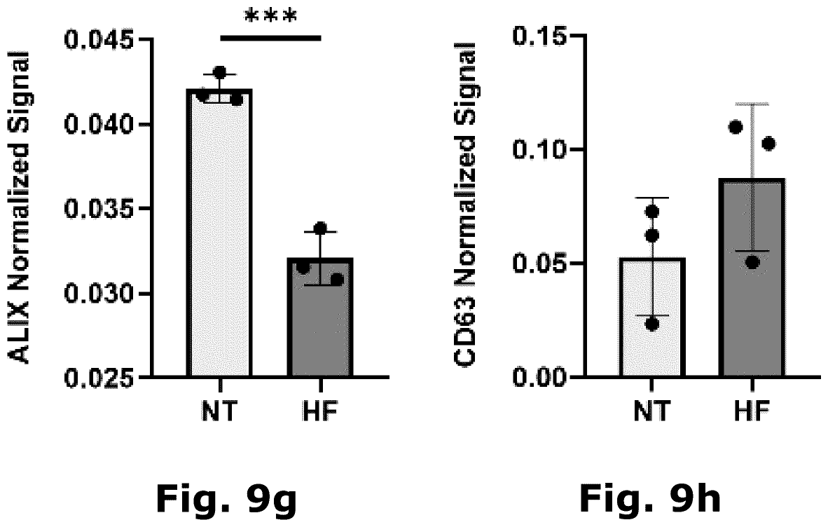

It was also found that fractal topography elicits enhanced signs of cell polarity. Apical surfaces of epithelial cells are decorated with glycans that contribute to regulation of cell structure, function, and communication via protruding forms such as charged villi, blebs, and secreted extracellular vesicles (EVs).^{[A19]} It was hypothesized that the arrangement of ECM proteins, facilitated by fractal topographical cues, could mediate assembly of podocyte structures involved in polarization. SEM underscored the interdigitated 3D morphology of cells on fractal substrates vs flat and RT controls (FIG. 9a). The protruding apical structures appeared to progress from rounded (mushroom regime) in the NT group to elongated finger-like protrusions (brush regime) in the HF group, according to the glycocalyx development model by Shurer et al. (FIG. 9a).^{[A19]}

Fractal topography was critical for podocyte marker localization, as confocal z-stacks demonstrated greater portions of podocin localization on top of the topography in the fractal groups (FIG. 9b) as well as greater presence of podocin in the fractal groups. Podocalyxin, a negatively charged sialylated glycoprotein involved in polarization^{[A20]} and basal organization,^{[A21]} exhibited stable protein expression and an increasing trend in the HF group (FIGS. 9c-d).

Epithelial polarity is often associated with a dense glycocalyx,^{[A21],[A22]} and a dense glycocalyx is likewise associated with the budding of extracellular vesicles (EV).^{[A19]} TEM revealed the cup-like shape of EV's (FIG. 9e) and expression of EV markers ALIX and CD63 (FIGS. 9f-h). Although both are exosome markers, ALIX exhibited decreased expression on HF substrates while high ALIX has been reported in exosomes of certain cancers.^{[A23],[A24]} Collectively, these observations point to the signs of physiological relevance of podocyte apical surface morphology on fractal substrates.^{[A19],[A25]}

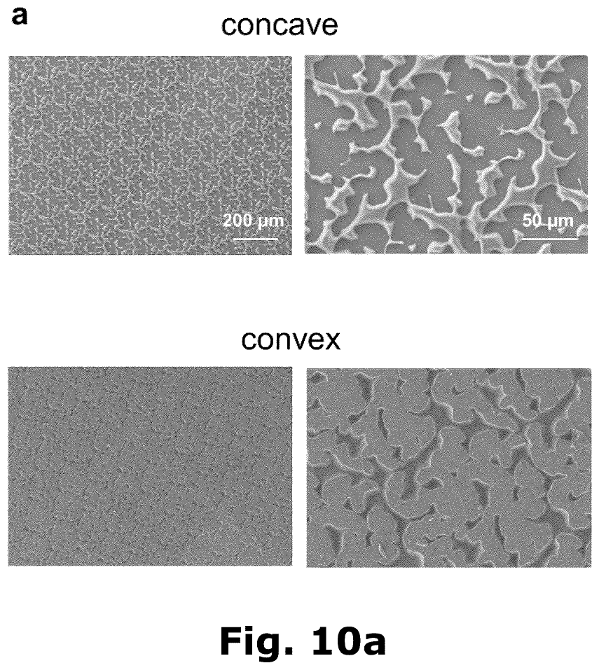

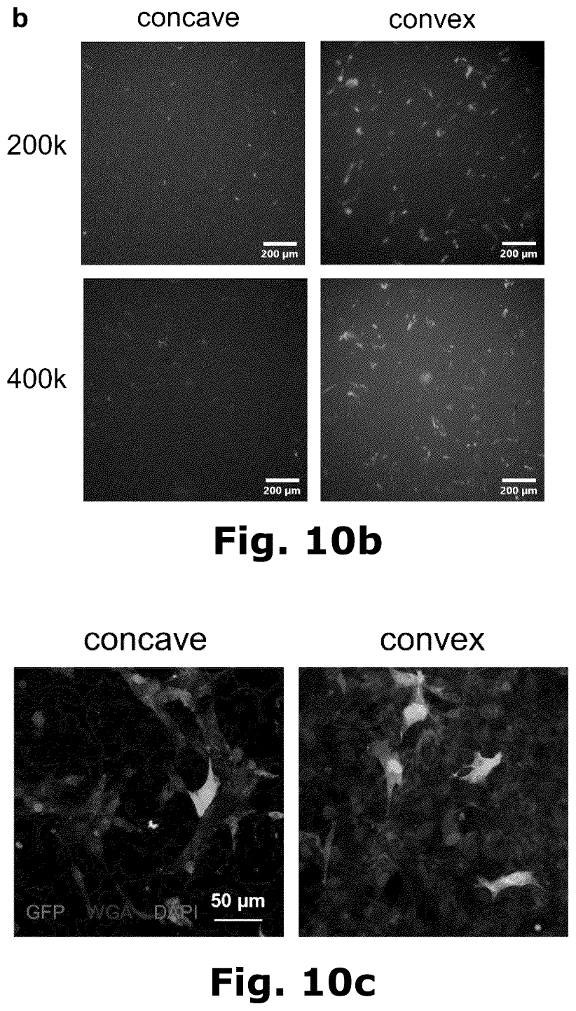

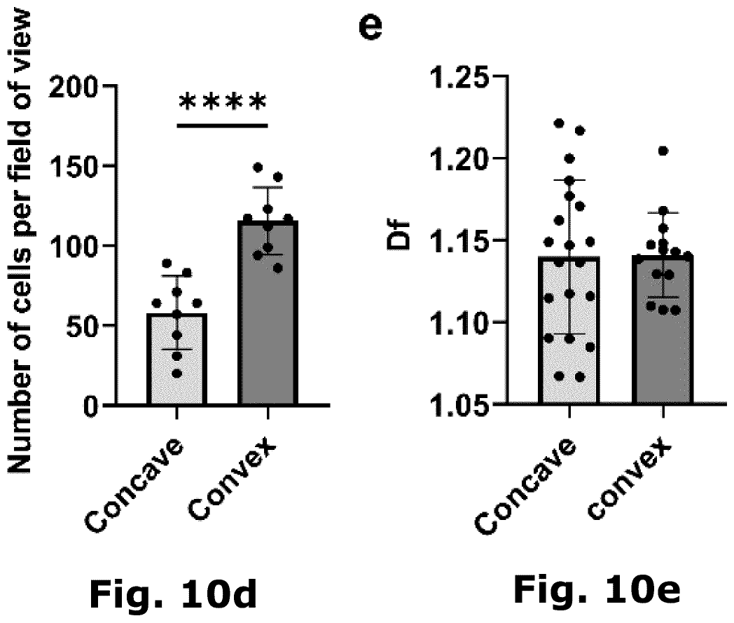

Mosaic assay revealed single cell morphology of podocytes cultured in vitro: It has been a challenge to observe podocyte morphology in vitro due to the difficulty in delineating the border of a single cell from those of its neighbors. To visualize single cell morphology in a confluent layer of podocytes cultured in vitro, optically silent podocytes were mixed with their counterpart labelled with green fluorescent protein (GFP) to obtain a mixed cell population with approximately 1/12 fluorescent cells. This mixed cell population is termed Mosaic assay, which enables single cell morphology to be easily visualized under fluorescence (FIG. 10c). Using Mosaic array, fractal dimension of podocyte morphology, an indicator of cell maturation and function, can be analyzed via the box counting method.

Furthermore, it was attempted to minimize the space in between slices of HF topography as it was those biophysical cues that drove the increased expression of podocyte markers and polarity observed. The HF patterns for the master mould were modified by more densely packing the HF design unit in order to reduce the flat region in between the HF design units. Since it is straightforward to create concave and convex patterns by micromoulding (FIG. 10a), it was tested whether the direction of the fractal topography affected podocyte cultivation using the Mosaic assay. Interestingly, cell coverage was significantly higher on convex versus concave patterns (FIGS. 10b-d) despite comparable cell morphology Df (FIG. 10e).

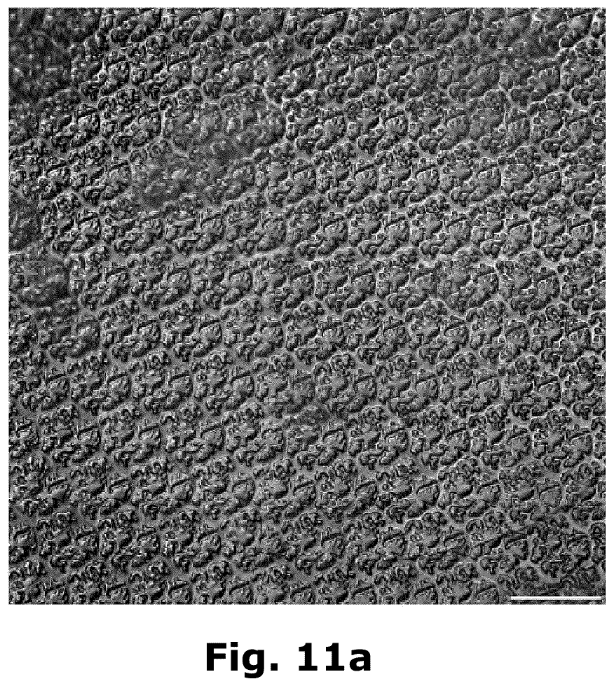

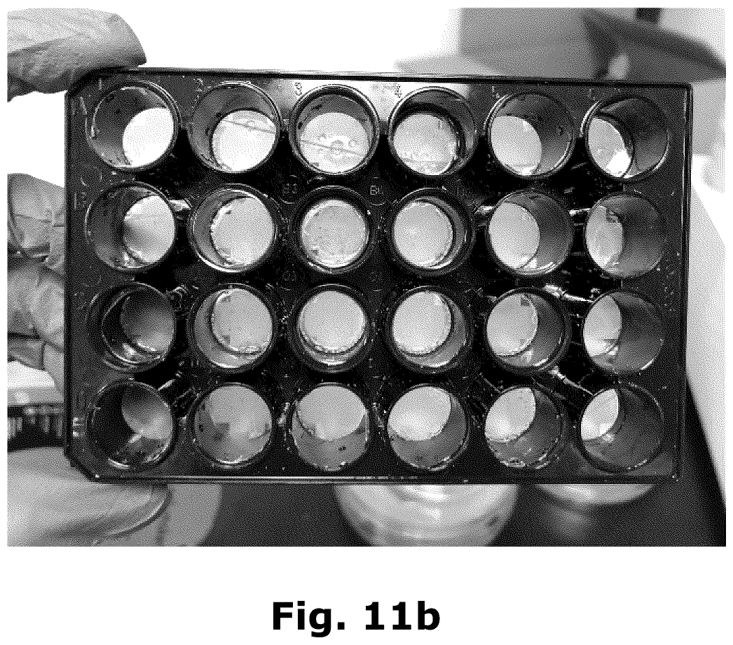

FIGS. 11a-c show polystyrene substrates with high density HF patterns. To eliminate the use of PDMS for scaleable production of the fractal topographical substrates, it was necessary to demonstrate podocyte branching morphology on substrates prepared via hot embossing of tissue culture polystyrene. The fractal features were clearly imprinted (FIG. 11a) and successfully assembled with a bottomless well plate for cell culture (FIG. 11b). Mosaic human podocytes were able to adhere and grow on polystyrene substrates coated with Matrigel (FIG. 11c). The use of thermoplastic materials enabled us to rapidly fabricate high density fractal patterns covering the entire well in a 24-well format (FIG. 11b).

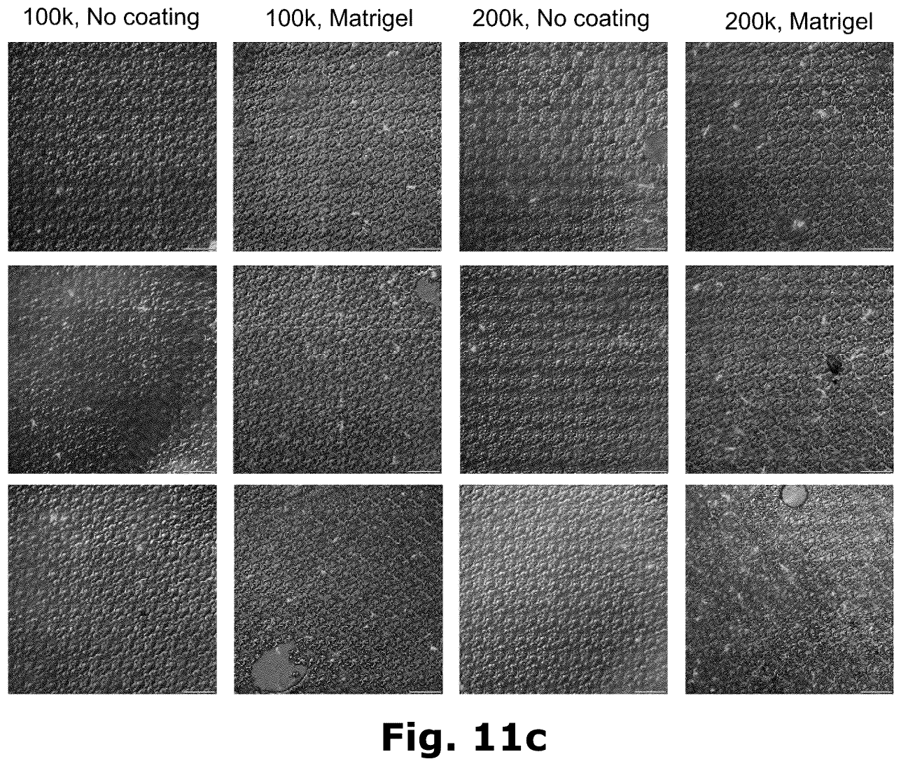

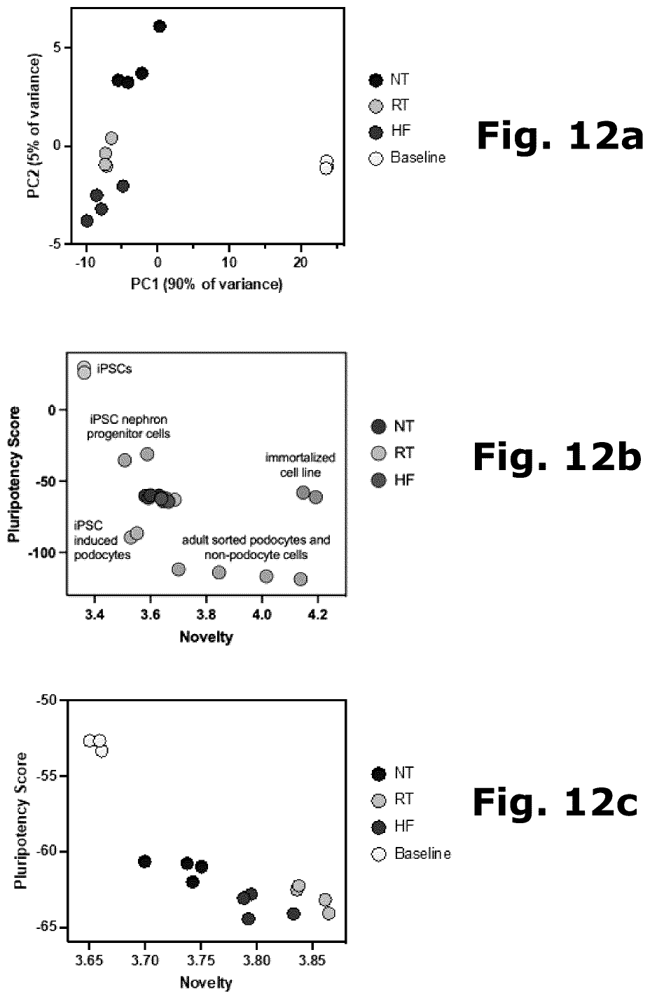

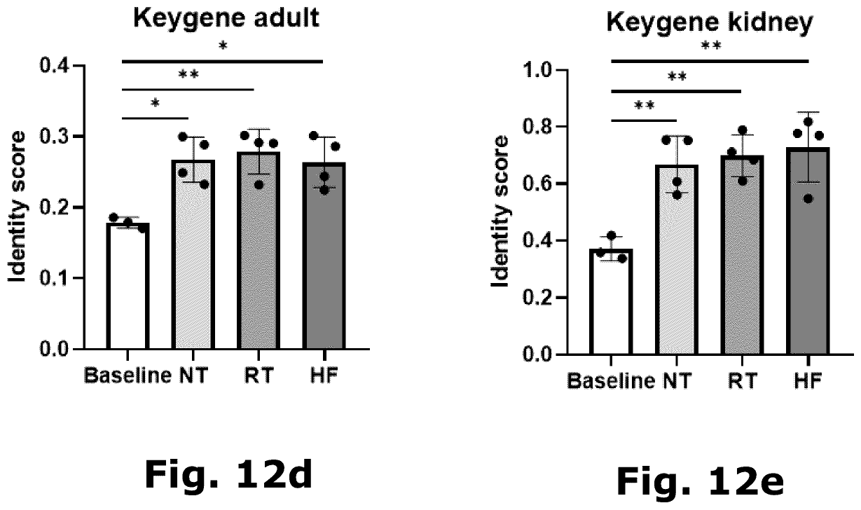

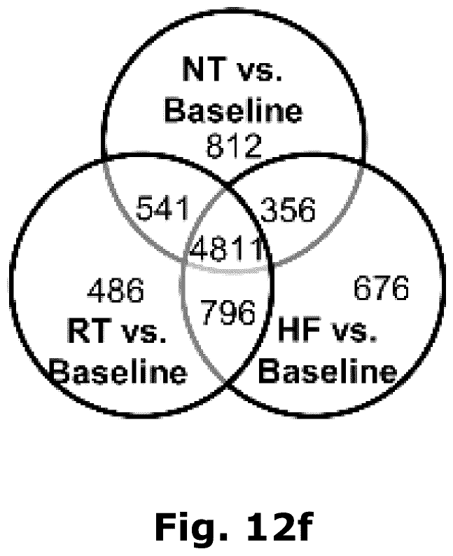

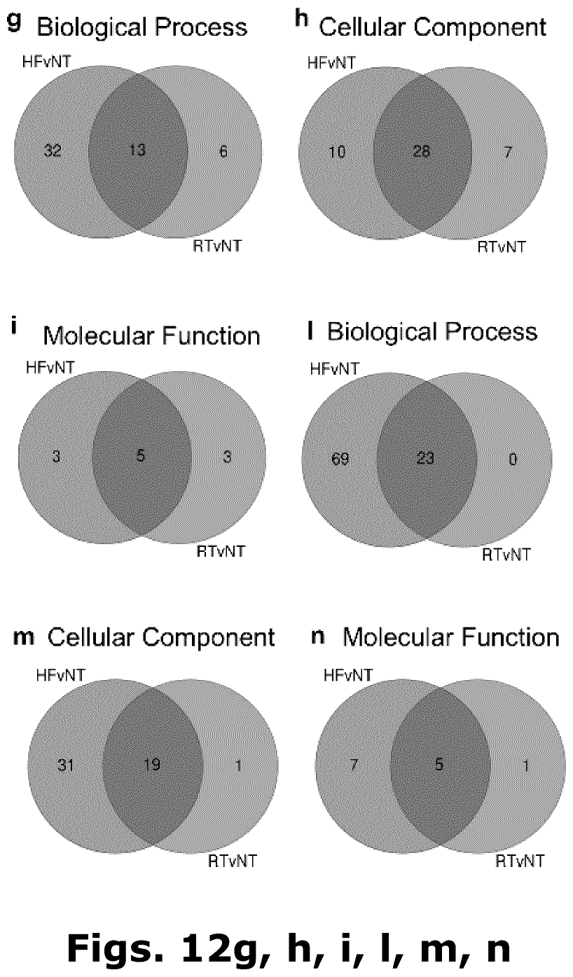

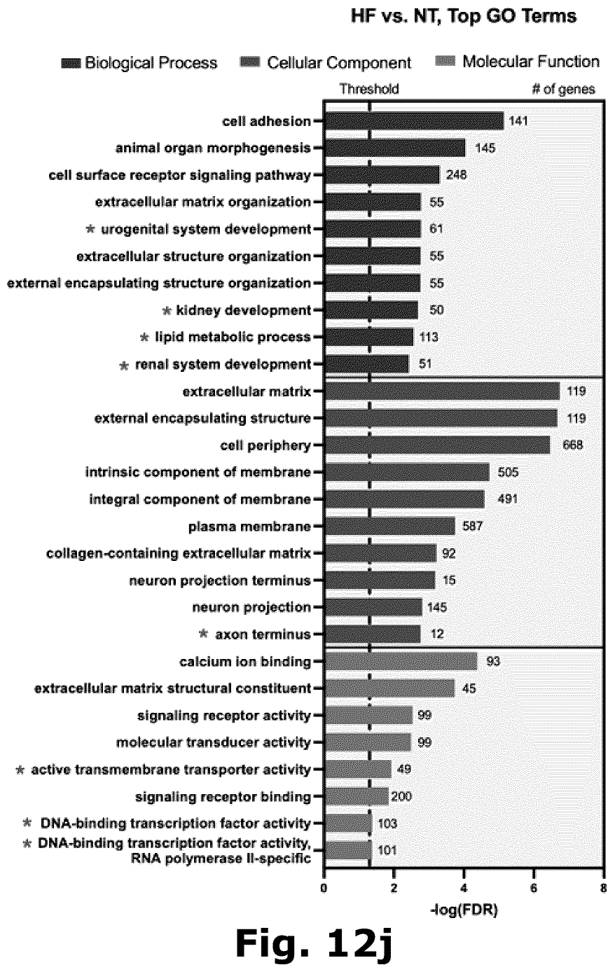

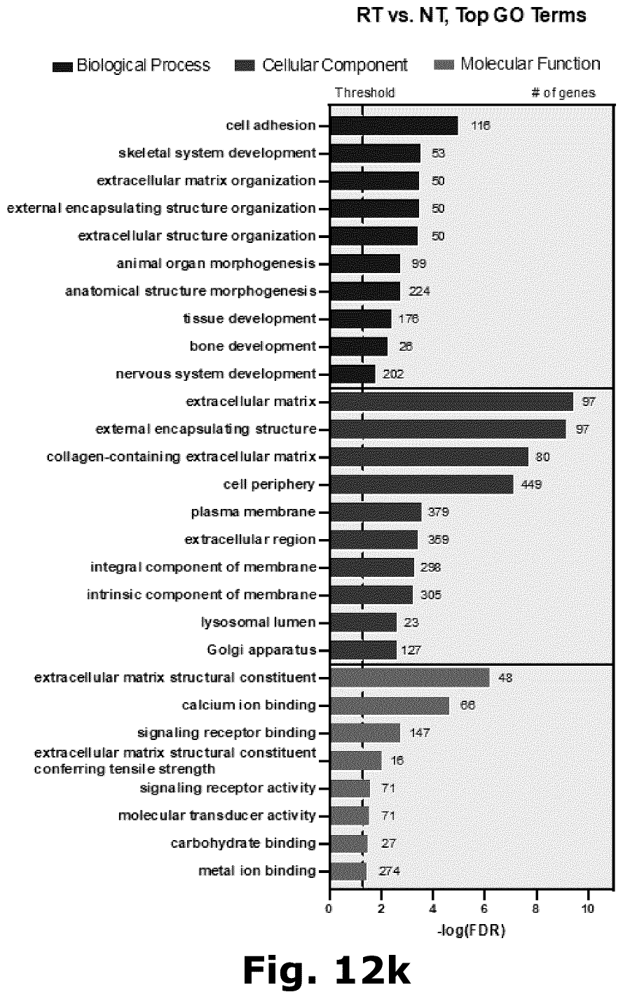

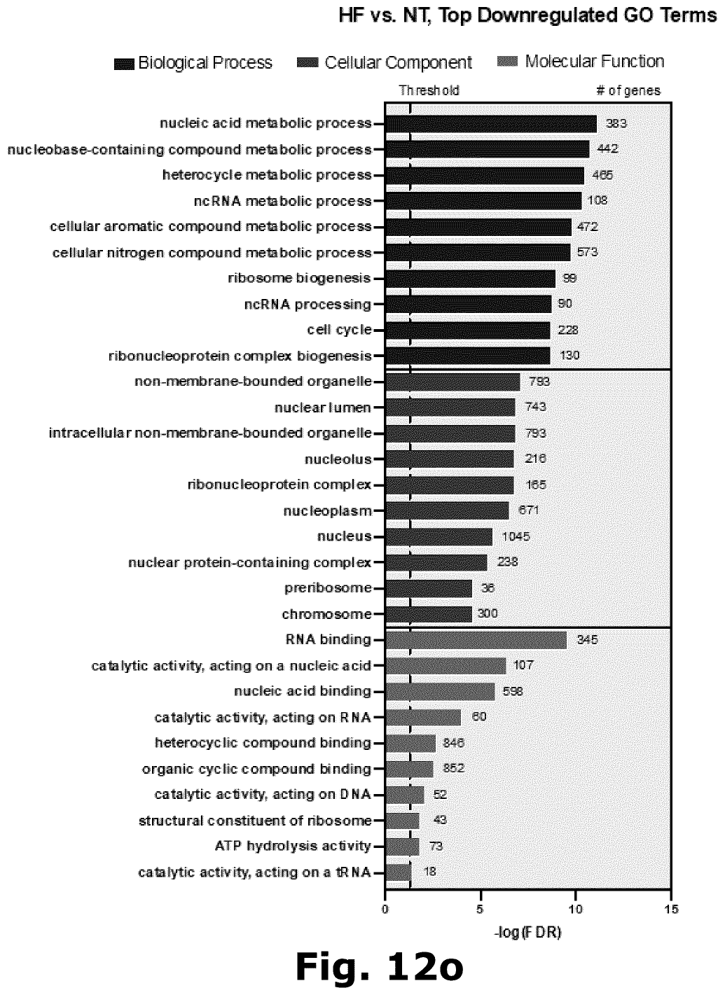

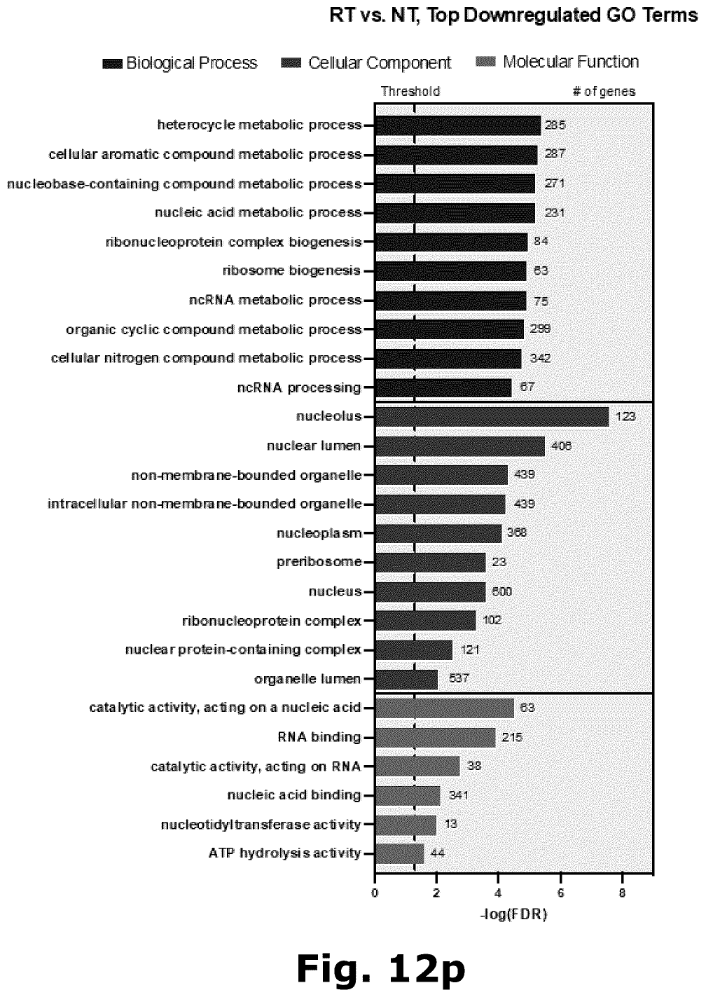

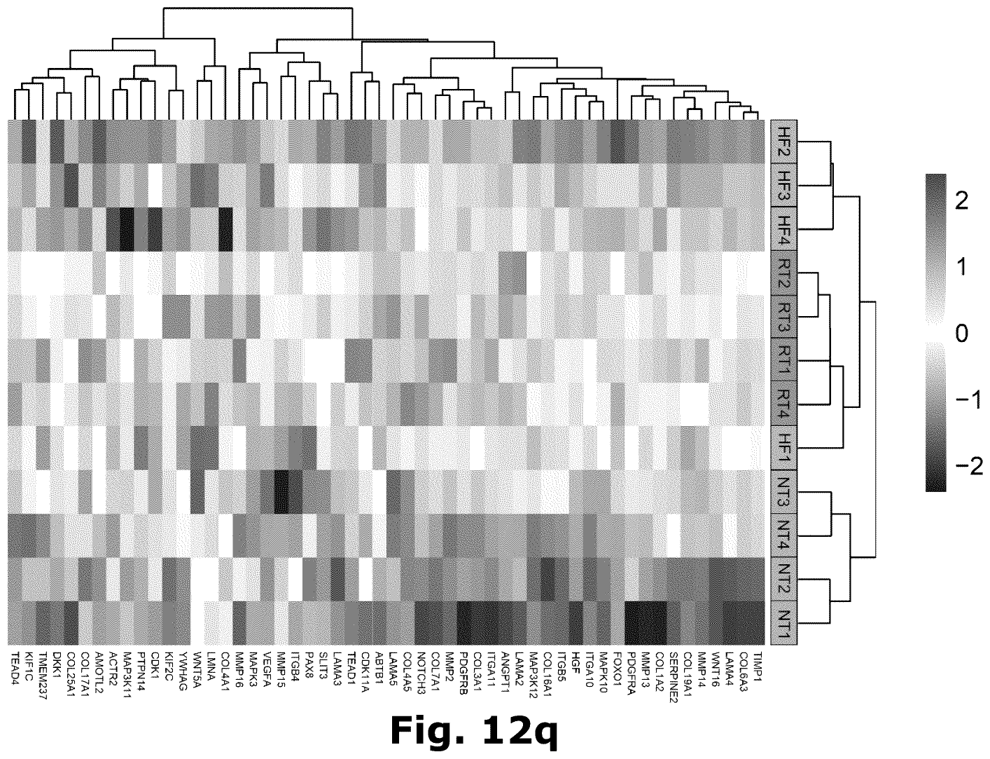

FIGS. 12a-q relate to RNA sequencing which suggests that fractal topography modulates podocyte response via ECM deposition, cell adhesion and kidney development. Distinct gene expression profiles suggest enhanced ECM deposition, remodelling and signal transduction on fractal topographical substrates: To explore the changes of cellular transcriptome that possibly drove the effect observed on fractal topography, RNA sequencing data^{[A26]} from primary fetal human podocytes was analyzed, which is a more authentic cell source than cell lines, grown on NT, RT, and HF substrates as well as cells prior to culture considered as the baseline. The data is published and available on GEO repository under accession code GSE185491, where frozen indicates baseline cells. Not only did cells cultured on the three substrate groups effectively clustered and against baseline cells by unsupervised principal component analysis (PCA) (FIG. 12a), two analyses, PluriTest (FIGS. 12b-c) and Keygenes (FIGS. 12d-e), also confirmed a more mature age and a more definitive kidney identity of cells on fractal substrates and of Yoshimura et al.^{[A6]} datasets in comparison to the baseline cells. Baseline cells were substantially different from cultivated cells with additional differences emerging with topographical culture (i.e. unique 486 genes on RT and 676 on HF, FIG. 12f).

Next, most significantly enriched gene ontology (GO) terms among all genes that were significantly upregulated in HF vs NT and RT vs HT were looked at. The results indicated that topography in general resulted in upregulated cell adhesion and organ morphogenesis biological processes as well as upregulated ECM, cell periphery and plasma membrane cellular components (FIGS. 12g-k). Yet, when HF vs NT was contrasted to RT vs NT, only HF group exhibited upregulation in GO terms for kidney development, renal system development, regulation of anion transport, urogenital system development, cell communication, metanephros development, cell-cell adhesion, vesicle mediated transport and cell-cell signaling (red stars in FIG. 12j). On the other hand, the top 10 most significantly downregulated genes in the HF group compared to other groups, were related to metabolic process and cell cycle (FIGS. 12l-p), consistent with low proliferation levels known as the effect of topographical cues.^{[A27]}

The expression of a number of ECM molecules were examined, including integrin receptor subunits, mitogen activated kinase (MAPK) isoforms, matrix metalloproteinases (MMPs) and signal transduction molecules that exhibited significant differences between the HF group vs NT alone or between both HF vs NT and HF vs RT (FIG. 12q). Clearly, topographical substrates exhibited a significantly higher expression of key collagen and laminin isoforms (COL6A3, COL1A2, COL19A, COL16A1, LAMA2, COL3A1, COL7A1, COL4A5, LAMA5, LAMA3) with notably high expression in the HF condition, whereas COL17A, reported to be implicated in the growth of multi-layered transformed epithelium was downregulated in HF group.^{[28]} COL4A5 and LAMA subunits are key structural components of the glomerular basement membrane. COL4A5 in particular, is one of the critical determinants of glomerular basement membrane structural integrity and aberrant expression of this protein is linked to numerous glomerular kidney diseases, e.g. focal segmental glomerulosclerosis (FSGS), Alport's, diabetic nephropathy, etc. Key matrix remodelling enzymes were also upregulated on topography and generally higher in the HF group (TIMP1, MMP13, MMP14, MMP15, MMP16) along with integrin receptor subunits (ITGA10, ITGB5, ITGA11).

PAX8 which regulates branching morphogenesis was higher on HF substrates, and so was SLIT3, whose knockdown results in defective kidney development.^{[A29],[A30]} Differences in gene expression of important signal transduction proteins (e.g. NOTCH3, high in HF) and morphogens (e.g. WNT5A and DKK1 low in HF), transcription factors (e.g. YAP pathway molecule AMOTL2, low in HF) and kinases (MAPK3 significantly higher in both HF vs NT and HF vs RT) of relevance to glomerulus development and kidney function^{[A31],[A32]} presented with possible candidates for elucidation of biochemical drivers of the observed branching behaviour (FIG. 12q). These data also solidify topographical substrates, particularly HF substrate, as important determinants of podocyte maturity and differentiation.

Interestingly, the expression of angiogenic factors (VEGF, PDGFA, HGF, ANGPT1) was enhanced on topographical substrates, which may be important in future tissue engineering applications (FIG. 12q). For FIG. 12q, RNA sequencing data from primary GW18 fetal human podocytes after 3 days in culture on n=4 scaffold samples per group and baseline cells prior to culture available on GEO repository under accession code GSE185491 was used for analysis.^{[A26]}

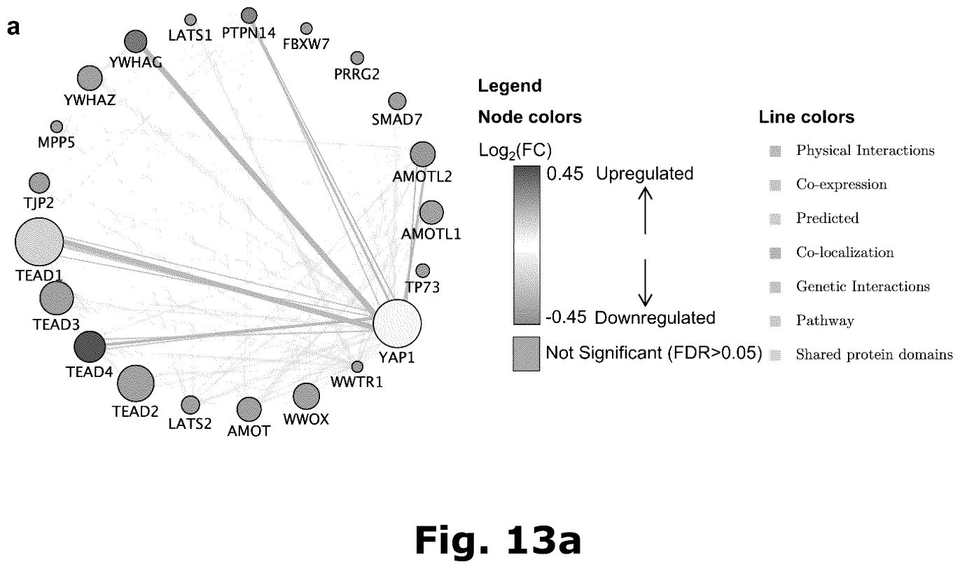

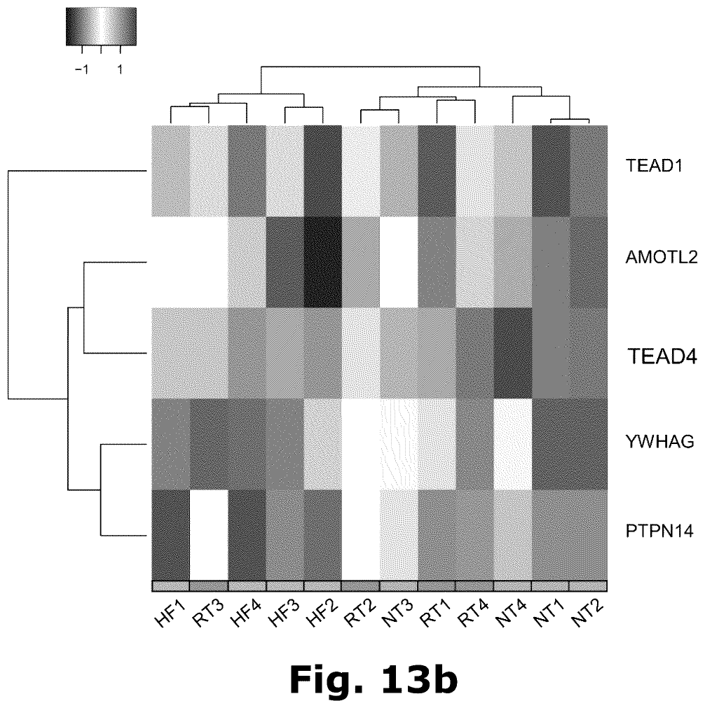

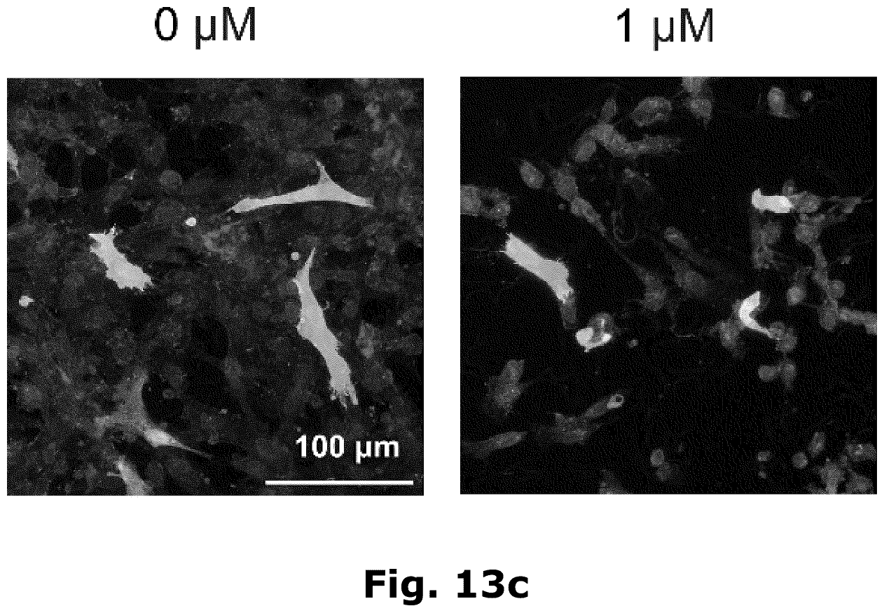

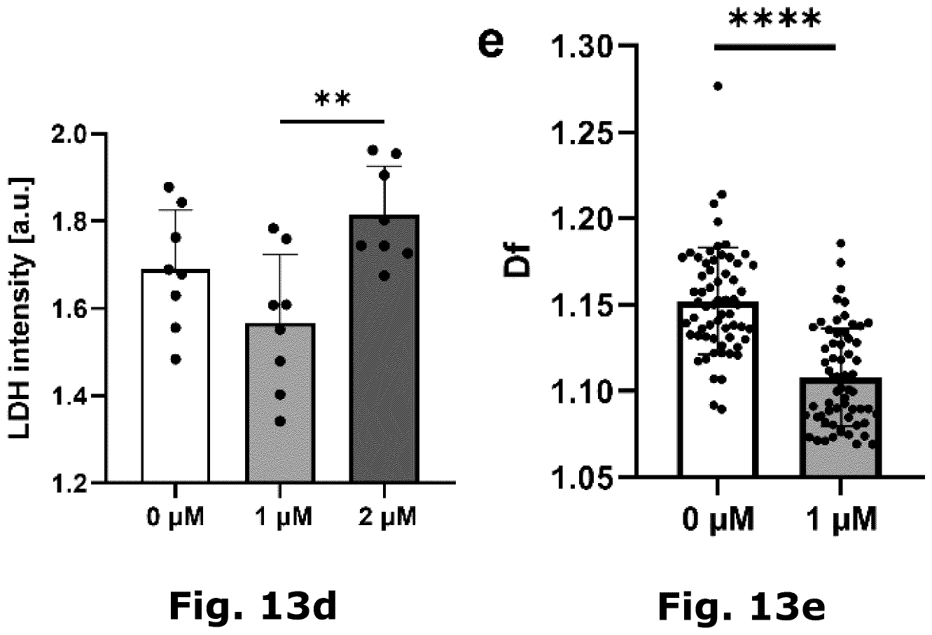

YAP signaling pathway plays a role in maintaining podocyte morphology. FIGS. 13a-e illustrate how YAP signaling pathway plays a role in maintaining podocyte morphology. RNA sequencing data from primary GW18 fetal human podocytes after 3 days in culture on n=4 scaffold samples per group and baseline cells prior to culture available on GEO repository under accession code GSE185491 was used for analysis.^{[A26]}. FIGS. 13a-b relate to primary fetal human podocytes, while FIGS. 13c-e relate to non-conditionally immortalized human podocyte cell line.

Upon searching the RNA sequencing data, key members of YAP1 interactome (generated using GeneMANIA algorithm^{[A33],[A34]}) were identified that were differentially regulated on the HF substrates in comparison to NT and RT (FIG. 13a-b). YAP interactome members were found to be differentially regulated on HF substrates (FIG. 13a). Blue nodes are genes of the YAP1 interactome that are downregulated in HF vs. NT. Red nodes are genes of the YAP1 interactome that are upregulated in HF vs. NT. Grey nodes are still part of the YAP1 interactome according the GeneMANIA algorithm, but had no significant difference between HF and NT from the RNA seq data. The lines connecting nodes describe the types of interactions between the nodes.

TEAD1^{[A35]} which is primarily responsible for cell adhesion, and motility was upregulated in HF substrates, whereas TEAD4 which is known to supress collagen 1 promoter activity^{[A36]} was downregulated on HF consistent with enhanced matrix deposition and remodelling in this group (FIGS. 13a-b). YWHAG which is primarily responsible for proliferation was downregulated on HF, consistent with its effect of reduced proliferation. AMOT family (downregulated AMOTL2 in HF), inhibits YAP1 by regulating its localization and promoting its phosphorylation,^{[A37]} while PTPN14 which was downregulated in HF, regulates YAP intracellular localization and inhibits its transcriptional co-activator activity.^{[A38]} These findings suggest that YAP1 activity may be responsible for the observed effects of cellular branching.

Upon YAP inhibition in human podocyte cell line on HF substrates (FIG. 13c) in the presence of suppressor of YAP-TEAD complex (Verteporfin^{[A39]}) at the dose of 1 μM, which did not show any significant deterioration of cell viability (FIG. 13d), Df of podocyte morphology was decreased significantly in comparison to the inhibitor-free control (FIG. 13e). Collectively, these results support the involvement of YAP in cell shape control on fractal substrates.

Overall, this study illustrates that the fractal nature of glomerular podocytes and the glomerulus itself, helps to contribute to the functional identification of healthy vs. diseased states. The findings suggest how fractal shape cues could drive cell maturation: fractal topography organizes ECM protein adhesion and subsequent cell clustering into precise geometries, to facilitate assembly and localization of subcellular structures, resulting in higher-order branching morphology and polarity. This behaviour was confirmed by using or considering three cell sources: conditionally immortalized mouse podocyte line, non-conditionally immortalized human podocyte line, and primary human fetal podocytes, as each source offers differences in terms of proliferation rate, genetic fidelity, and physiological relevance that are important for the validation process.

YAP activation, through nuclear localization is required for podocyte function,^{[A40]} as well as nephron development, as conditional knockdown of YAP in mouse kidney led to reduced nephrogenesis and morphogenesis, in a manner that was independent of proliferation and apoptosis,^{[A41]} consistent with the observation of YAP1 involvement in shape patterning.

This work captured some of the fractal effects by fitting a single power law to morphologies of podocytes and glomeruli that are in reality multi-fractal.

Podocyte cell sources: In this second example study, three podocyte cell sources were used or considered: (1) conditionally immortalized murine podocyte E11 cell line (referred to as mouse podocytes); (2) human non-conditionally immortalized Podo/tert 256 cell line (referred to as human podocytes); and (3) primary human fetal podocytes from gestation week 18 (referred to as primary human podocytes). Timepoints were different for mouse and human experiments due to the different nature of the cell lines. Each cell source that was used has strengths and weaknesses.

The mouse cell line was conditionally immortalized, thus it has a thermoswitching mechanism that could deactivate the forced cell cycle processes and slow proliferation and allow differentiation over longer cultivation periods, namely, of at least 14 days.

The non-conditionally immortalized human cells do not have a mechanism built-in for switching off the genetically induced immortalization. These cells keep proliferating past confluence and this limits the ability to achieve a fully differentiated phenotype. While they represent a human source, their cultivation period is short before cells begin overcrowding. On topographical substrates, the cultures could only last ˜5 days before overgrowing since they do not have a thermoswitching capability on the immortalization. As the cells start growing on top of one another, they are prone to being peeled off from the PDMS substrates during handling of the scaffolds.

The primary cells are the most authentic and unmodified cell source, however, limited in number and more immature by definition as they are fetal-derived. In this case, the RNA sequencing data used for analysis was generated from podocytes from gestation week 18 via a commercial source (Lonza). At this stage of development, podocytes are still relatively immature and able to undergo proliferation as they are still developing and populating the developing nephron and glomerulus. The manufacturer provides these fetal cells due to their still-present proliferative capacity that allows them to be used in culture for limited passages. Thus, it is expected that the primary fetal cells undergo limited proliferation, as is indeed provided and validated by the manufacturer.

By considering all three cell sources, this study has a more balanced view on the biophysical phenomena observed in the system.

The embodiments of the present disclosure described above are intended to be examples only. The present disclosure may be embodied in other specific forms. Alterations, modifications and variations to the disclosure may be made without departing from the intended scope of the present disclosure. While the systems, devices and processes disclosed and shown herein may comprise a specific number of elements/components, the systems, devices and assemblies could be modified to include additional or fewer of such elements/components. For example, while any of the elements/components disclosed may be referenced as being singular, the embodiments disclosed herein could be modified to include a plurality of such elements/components. Selected features from one or more of the above-described embodiments may be combined to create alternative embodiments not explicitly described. All values and sub-ranges within disclosed ranges are also disclosed. The subject matter described herein intends to cover and embrace all suitable changes in technology. All references mentioned are hereby incorporated by reference in their entirety.

### REFERENCES

1. Sornette, D. Critical Phenomena in Natural Sciences: Chaos, Fractals, Selforganization and Disorder: Concepts and Tools. (Springer Science & Business Media, 2006).
2. Khan, J. A. et al. Fetal liver hematopoietic stem cell niches associate with portal vessels. Science 351, 176-80 (2016).
3. Korolj, A., Wu, H.-T. & Radisic, M. A healthy dose of chaos: Using fractal frameworks for engineering higher-fidelity biomedical systems. Biomaterials 219, 119363 (2019).
4. Ichimura, K. et al. Morphological process of podocyte development revealed by block-face scanning electron microscopy. J Cell Sci 130, 132-142 (2017).
5. Kriz, W., Shirato, I., Nagata, M., LeHir, M. & Lemley, K. V. The podocyte's response to stress: the enigma of foot process effacement. American Journal of Physiology—Renal Physiology 304, F333-F347 (2012).
6. Shankland, S. J., Pippin, J. W., Reiser, J. & Mundel, P. Podocytes in culture: past, present, and future. Kidney International 72, 26-36 (2007).
7. Yoshimura, Y. et al. Manipulation of Nephron-Patterning Signals Enables Selective Induction of Podocytes from Human Pluripotent Stem Cells. JASN 30, 304-321 (2019).
8. Takasato, M. et al. Kidney organoids from human iPS cells contain multiple lineages and model human nephrogenesis. Nature 526, 564-8 (2015).
9. Friedrich, C., Endlich, N., Kriz, W. & Endlich, K. Podocytes are sensitive to fluid shear stress in vitro. American Journal of Physiology—Renal Physiology 291, F856 (2006).
10. Petrosyan, A. et al. A glomerulus-on-a-chip to recapitulate the human glomerular filtration barrier. Nat Commun 10, 3656 (2019).
11. Musah, S. et al. Mature induced-pluripotent-stem-cell-derived human podocytes reconstitute kidney glomerular-capillary-wall function on a chip. Nature Biomedical Engineering 1, 0069 (2017).
12. Hu, M. et al. A biomimetic gelatin-based platform elicits a pro-differentiation effect on podocytes through mechanotransduction. Scientific reports 7, 43934 (2017).
13. Xie, R. et al. h-FIBER: Microfluidic Topographical Hollow Fiber for Studies of Glomerular Filtration Barrier. ACS Cent. Sci. (2020) doi:10.1021/acscentsci.9b01097.
14. Korolj, A. et al. Curvature facilitates podocyte culture in a biomimetic platform. Lab on a Chip 18, 3112-3128 (2018).
15. Goldberger, A. L. et al. Fractal dynamics in physiology: Alterations with disease and aging. PNAS 99, 2466-2472 (2002).
16. Barros, G. O., Navarro, B., Duarte, A. & dos-Santos, W. L. C. PathoSpotter-K: A computational tool for the automatic identification of glomerular lesions in histological images of kidneys. Sci Rep 7, 46769 (2017).
17. Clotet-Freixas, S. et al. Extracellular Matrix Injury of Kidney Allografts in Antibody-Mediated Rejection: A Proteomics Study. JASN (2020) doi:10.1681/ASN.2020030286.
18. Cheng, H., Dong, H. R., Lin, R. Q., Sun, L. J. & Chen, Y. P. Determination of normal value of glomerular size in Chinese adults by different measurement methods. Nephrology (Carlton) 17, 488-92 (2012).
19. Steinhausen, M., Snoei, H., Parekh, N., Baker, R. & Johnson, P. C. Hydronephrosis: A new method to visualize vas afferens, efferens, and glomerular network. Kidney International 23, 794-806 (1983).
20. Freudenberg, U. et al. Electrostatic Interactions Modulate the Conformation of Collagen I. Biophys J 92, 2108-2119 (2007).
21. Uquillas, J. A. & Akkus, O. Modeling the Electromobility of Type-I Collagen Molecules in the Electrochemical Fabrication of Dense and Aligned Tissue Constructs. Ann Biomed Eng 40, 1641-1653 (2012).
22. Cross, D. J. When the charge on a planar conductor is a function of its curvature. Journal of Mathematical Physics 55, 123504 (2014).
23. Bannerman, A. D., Lu, R. X. Z., Korolj, A., Kim, L. H. & Radisic, M. The use of microfabrication technology to address the challenges of building physiologically relevant vasculature. Current Opinion in Biomedical Engineering (2017).
24. Pavenstädt, H., Kriz, W. & Kretzler, M. Cell Biology of the Glomerular Podocyte. Physiological Reviews 83, 253-307 (2003).
25. Nielsen, J. S. et al. The CD34-Related Molecule Podocalyxin Is a Potent Inducer of Microvillus Formation. PLoS ONE 2, e237 (2007).
26. Economou, C. G. Enhanced podocalyxin expression alters the structure of podocyte basal surface. Journal of Cell Science 117, 3281-3294 (2004).
27. Grgic, I. et al. Imaging of Podocyte Foot Processes by Fluorescence Microscopy. JASN 23, 785-791 (2012).
28. Siegerist, F. et al. Structured illumination microscopy and automatized image processing as a rapid diagnostic tool for podocyte effacement. Scientific Reports 7, (2017).
29. Shurer, C. R. et al. Physical Principles of Membrane Shape Regulation by the Glycocalyx. Cell 177, 1757-1770.e21 (2019).
30. Dingal, P. C. D. P. et al. Fractal heterogeneity in minimal matrix models of scars modulates stiff-niche stem-cell responses via nuclear exit of a mechanorepressor. Nat Mater 14, 951-960 (2015).
31. Burger, D. et al. Urinary Podocyte Microparticles Identify Prealbuminuric Diabetic Glomerular Injury. J Am Soc Nephrol 25, 1401-1407 (2014).
32. Park, J. et al. Single-cell transcriptomics of the mouse kidney reveals potential cellular targets of kidney disease. Science 360, 758-763 (2018).
33. Motavalli, R. et al. The lethal internal face of the coronaviruses: Kidney tropism of the SARS, MERS, and COVID19 viruses. IUBMB Life 73, 1005-1015 (2021).
34. Martinez-Rojas, M. A., Vega-Vega, O. & Bobadilla, N. A. Is the kidney a target of SARS-CoV-2? Am J Physiol Renal Physiol 318, F1454-F1462 (2020).
35. Ye, M. et al. Glomerular Localization and Expression of Angiotensin-Converting Enzyme 2 and Angiotensin-Converting Enzyme: Implications for Albuminuria in Diabetes. JASN 17, 3067-3075 (2006).
36. Velez, J. C. Q. The importance of the intrarenal renin-angiotensin system. Nat Rev Nephrol 5, 89-100 (2009).
37. Okuda, S. et al. Adriamycin-induced nephropathy as a model of chronic progressive glomerular disease. Kidney Int 29, 502-510 (1986).
38. Savin, V. J. et al. Circulating Factor Associated with Increased Glomerular Permeability to Albumin in Recurrent Focal Segmental Glomerulosclerosis. New England Journal of Medicine 334, 878-883 (1996).
39. Srivastava, P. et al. Development of a novel cell-based assay to diagnose recurrent focal segmental glomerulosclerosis patients. Kidney International 95, 708-716 (2019).
40. Villasante, A. et al. Human Serum Enhances Biomimicry of Engineered Tissue Models of Bone and Cancer. Front Bioeng Biotechnol 9, 658472 (2021).
41. T. Ekvall, M. et al. Nanoplastics formed during the mechanical breakdown of daily-use polystyrene products. Nanoscale Advances 1, 1055-1061 (2019).
42. Shivaraju, M. et al. Airway stem cells sense hypoxia and differentiate into protective solitary neuroendocrine cells. Science 371, 52-57 (2021).
43. Shojaie, S. et al. Acellular Lung Scaffolds Direct Differentiation of Endoderm to Functional Airway Epithelial Cells: Requirement of Matrix-Bound HS Proteoglycans. Stem Cell Reports 4, 419-430 (2015).
44. Sage, A. T. et al. Fractal circuit sensors enable rapid quantification of biomarkers for donor lung assessment for transplantation. Science advances 1, e1500417 (2015).
45. Ichimura, K. et al. Three-dimensional architecture of podocytes revealed by block-face scanning electron microscopy. Scientific reports 5, 8993 (2015).
46. Wagner, R. et al. High-resolution imaging of kidney vascular corrosion casts with Nano-CT. Microsc Microanal 17, 215-9 (2011).
47. The University of Utah Eccles Health Sciences Library. The Internet Pathology Laboratory for Medical Education—Renal Pathology. https://webpath.med.utah.edu/RENAHTML/RENAL101.html.
48. Sader, J. E., Lu, J. & Mulvaney, P. Effect of cantilever geometry on the optical lever sensitivities and thermal noise method of the atomic force microscope. Review of Scientific Instruments 85, 113702 (2014).
49. Sader, J. E. et al. Spring constant calibration of atomic force microscope cantilevers of arbitrary shape. Rev Sci Instrum 83, 103705 (2012).
50. Li, H. & Durbin, R. Fast and accurate short read alignment with Burrows-Wheeler transform. Bioinformatics 25, 1754-1760 (2009).
51. Li, H. & Durbin, R. Fast and accurate long-read alignment with Burrows-Wheeler transform. Bioinformatics 26, 589-595 (2010).
52. Li, H. Aligning sequence reads, clone sequences and assembly contigs with BWA-MEM. arXiv:1303.3997 [q-bio] (2013).
53. Anders, S., Pyl, P. T. & Huber, W. HTSeq—A Python framework to work with high-throughput sequencing data. 002824 https://www.biorxiv.org/content/10.1101/002824v2 (2014) doi:10.1101/002824.
54. Love, M. I., Huber, W. & Anders, S. Moderated estimation of fold change and dispersion for RNA-seq data with DESeq2. Genome Biology 15, 550 (2014).
55. Kolde, R. pheatmap: Pretty Heatmaps. (2019).
56. Ge, S. X., Jung, D. & Yao, R. ShinyGO: a graphical gene-set enrichment tool for animals and plants. Bioinformatics 36, 2628-2629 (2020).
57. Famure, O., Phan, N. A.-T. & Kim, S. J. Health information management for research and quality assurance: the Comprehensive Renal Transplant Research Information System. Healthc Manage Forum 27, 30-36 (2014).

### SUPPLEMENTARY REFERENCES

S1. Ichimura, K. et al. Morphological process of podocyte development revealed by block-face scanning electron microscopy. J Cell Sci 130, 132-142 (2017).
S2. Siegerist, F. et al. Structured illumination microscopy and automatized image processing as a rapid diagnostic tool for podocyte effacement. Scientific Reports 7, (2017).
S3. Lausecker, F. et al. Vinculin is required to maintain glomerular barrier integrity. Kidney International 93, 643-655 (2018).
S4. Clotet-Freixas, S. et al. Extracellular Matrix Injury of Kidney Allografts in Antibody-Mediated Rejection: A Proteomics Study. JASN (2020) doi:10.1681/ASN.2020030286.
S5. Korolj, A. et al. Curvature facilitates podocyte culture in a biomimetic platform. Lab on a Chip 18, 3112-3128 (2018).
S6. Park, J. et al. Single-cell transcriptomics of the mouse kidney reveals potential cellular targets of kidney disease. Science 360, 758-763 (2018).
S7. Yoshimura, Y. et al. Manipulation of Nephron-Patterning Signals Enables Selective Induction of Podocytes from Human Pluripotent Stem Cells. JASN 30, 304-321 (2019).
S8. Saran, R. et al. US Renal Data System 2016 Annual Data Report: Epidemiology of Kidney Disease in the United States. Am J Kidney Dis 69, A7-A8 (2017).
S9. D'Agati, V. D., Kaskel, F. J. & Falk, R. J. Focal segmental glomerulosclerosis. N Engl J Med 365, 2398-2411 (2011).
S10. Tejani, A. & Stablein, D. H. Recurrence of focal segmental glomerulosclerosis posttransplantation: a special report of the North American Pediatric Renal Transplant Cooperative Study. J Am Soc Nephrol 2, S258-263 (1992).
S11. Uffing, A. et al. Recurrence of FSGS after Kidney Transplantation in Adults. Clin J Am Soc Nephrol 15, 247-256 (2020).

### ADDITIONAL REFERENCES

[A1] K. Ichimura, S. Kakuta, Y. Kawasaki, T. Miyaki, T. Nonami, N. Miyazaki, T. Nakao, S. Enomoto, S. Arai, M. Koike, K. Murata, T. Sakai, Journal of Cell Science 2017, 130, 132.
[A2] E. Jordanova, R. Jankovic, R. Naumovic, D. Celic, B. Ljubicic, S. Simic-Ogrizovic, G. Basta-Jovanovic, J Pathol Inform 2022, 13, 100108.
[A3] K. Kojima, A. Davidovits, H. Poczewski, B. Langer, S. Uchida, K. Nagy-Bojarski, A. Hovorka, R. Sedivy, D. Kerjaschki, Journal of the American Society of Nephrology 2004, 15, 2079.
[A4] A. Korolj, H.-T. Wu, M. Radisic, Biomaterials 2019, 219, 119363.
[A5] S. J. Shankland, J. W. Pippin, J. Reiser, P. Mundel, Kidney International 2007, 72, 26.
[A6] Y. Yoshimura, A. Taguchi, S. Tanigawa, J. Yatsuda, T. Kamba, S. Takahashi, H. Kurihara, M. Mukoyama, R. Nishinakamura, JASN 2019, 30, 304.
[A7] L. J. Hale, S. E. Howden, B. Phipson, A. Lonsdale, P. X. Er, I. Ghobrial, S. Hosawi, S. Wilson, K. T. Lawlor, S. Khan, A. Oshlack, C. Quinlan, R. Lennon, M. H. Little, Nat Commun 2018, 9, 5167.
[A8] M. Hu, E. U. Azeloglu, A. Ron, K. H. Tran-Ba, R. C. Calizo, I. Tavassoly, S. Bhattacharya, G. Jayaraman, Y. Chen, V. Rabinovich, R. Iyengar, J. C. Hone, J. C. He, L. J. Kaufman, Scientific reports 2017, 7, 43934.
[A9] S. Musah, A. Mammoto, T. C. Ferrante, S. S. F. Jeanty, M. Hirano-Kobayashi, T. Mammoto, K. Roberts, S. Chung, R. Novak, M. Ingram, T. Fatanat-Didar, S. Koshy, J. C. Weaver, G. M. Church, D. E. Ingber, Nature Biomedical Engineering 2017, 1, 0069.
[A10] A. Petrosyan, P. Cravedi, V. Villani, A. Angeletti, J. Manrique, A. Renieri, R. E. De Filippo, L. Perin, S. Da Sacco, Nat Commun 2019, 10, 3656.
[A11] R. Xie, A. Korolj, C. Liu, X. Song, R. X. Z. Lu, B. Zhang, A. Ramachandran, Q. Liang, M. Radisic, ACS Cent. Sci. 2020, DOI 10.1021/acscentsci.9b01097.
[A12] A. Korolj, C. Laschinger, C. James, E. Hu, C. Velikonja, N. Smith, I. Gu, S. Ahadian, R. Willette, M. Radisic, B. Zhang, Lab on a Chip 2018, 18, 3112.
[A13] K. Ichimura, S. Kakuta, Y. Kawasaki, T. Miyaki, T. Nonami, N. Miyazaki, T. Nakao, S. Enomoto, S. Arai, M. Koike, K. Murata, T. Sakai, J Cell Sci 2017, 130, 132.
[A14] A. Korolj, P. Aggarwal, T. Cui, S. Song, L. Shamaei, N. Rafatian, A. Radisic, S. Rodriguez-Ramirez, C. Liu, M. Li, K. Wagner, E. Virlee, S. Clotet-Freixas, M. Sadrzadeh, T. Filleter, U. Broeckel, A. Konvalinka, M. Radisic, Tissue Engineering—Part A 2022, 359.
[A15] A. Korolj, Engineering the Fractal Properties of Podocytes, Thesis 2021.
[A16] R. Wagner, D. Van Loo, F. Hossler, K. Czymmek, E. Pauwels, L. Van Hoorebeke, Microscopy and Microanalysis 2011, 17, 215.
[A17] K. Ichimura, N. Miyazaki, S. Sadayama, K. Murata, M. Koike, K. Nakamura, K. Ohta, T. Sakai, Sci Rep 2015, 5, 8993.
[A18] G. O. Barros, B. Navarro, A. Duarte, W. L. C. dos-Santos, Sci Rep 2017, 7, 46769.
[A19] C. R. Shurer, J. C.-H. Kuo, L. M. Roberts, J. G. Gandhi, M. J. Colville, T. A. Enoki, H. Pan, J. Su, J. M. Noble, M. J. Hollander, J. P. O'Donnell, R. Yin, K. Pedram, L. Möckl, L. F. Kourkoutis, W. E. Moerner, C. R. Bertozzi, G. W. Feigenson, H. L. Reesink, M. J. Paszek, Cell 2019, 177, 1757.
[A20] J. S. Nielsen, M. L. Graves, S. Chelliah, A. W. Vogl, C. D. Roskelley, K. M. McNagny, PLoS ONE 2007, 2, e237.
[A21] C. G. Economou, Journal of Cell Science 2004, 117, 3281.
[A22] J. S. Nielsen, K. M. McNagny, JASN 2009, 20, 1669.
[A23] J. Yang, Y. Zhang, X. Gao, Y. Yuan, J. Zhao, S. Zhou, H. Wang, L. Wang, G. Xu, X. Li, P. Wang, X. Zou, D. Zhu, Y. Lv, S. Zhang, Frontiers in Oncology 2021, 11.
[A24] D. Duijvesz, K. E. Burnum-Johnson, M. A. Gritsenko, A. M. Hoogland, M. S. V. den Berg, R. Willemsen, T. Luider, L. Paša-Tolić, G. Jenster, PLOS ONE 2013, 8, e82589.
[A25] D. Burger, J.-F. Thibodeau, C. E. Holterman, K. D. Burns, R. M. Touyz, C. R. J. Kennedy, J Am Soc Nephrol 2014, 25, 1401.
[A26] “GEO Accession viewer,” can be found under https://www.ncbi.nlm.nih.gov/geo/query/acc.cgi?acc=GSE185491, n.d.
[A27] L. Qi, N. Li, R. Huang, Q. Song, L. Wang, Q. Zhang, R. Su, T. Kong, M. Tang, G. Cheng, PLOS ONE 2013, 8, e59022.
[A28] K. Kozawa, M. Sekai, K. Ohba, S. Ito, H. Sako, T. Maruyama, M. Kakeno, T. Shirai, K. Kuromiya, T. Kamasaki, K. Kohashi, S. Tanaka, S. Ishikawa, N. Sato, S. Asano, H. Suzuki, N. Tanimura, Y. Mukai, N. Gotoh, M. Tanino, S. Tanaka, K. Natsuga, T. Soga, T. Nakamura, Y. Yabuta, M. Saitou, T. Ito, K. Matsuura, M. Tsunoda, T. Kikumori, T. Iida, Y. Mizutani, Y. Miyai, K. Kaibuchi, A. Enomoto, Y. Fujita, Current Biology 2021, 31, 3086.
[A29] J. Liu, L. Zhang, D. Wang, H. Shen, M. Jiang, P. Mei, P. S. Hayden, J. R. Sedor, H. Hu, Mechanisms of Development 2003, 120, 1059.
[A30] M.-F. Wu, C.-Y. Liao, L.-Y. Wang, J. T. Chang, Tissue Barriers 2017, 5, e1331155.
[A31] C. Yuan, L. Ni, C. Zhang, X. Wu, Oxid Med Cell Longev 2020, 2020, 1809408.
[32] M. Bose, S. Almas, S. Prabhakar, Journal of Investigative Medicine 2017, 65, 1093.
[A33] D. Warde-Farley, S. L. Donaldson, O. Comes, K. Zuberi, R. Badrawi, P. Chao, M. Franz, C. Grouios, F. Kazi, C. T. Lopes, A. Maitland, S. Mostafavi, J. Montojo, Q. Shao, G. Wright, G. D. Bader, Q. Morris, Nucleic Acids Research 2010, 38, 214.
[A34] P. Shannon, A. Markiel, O. Ozier, N. S. Baliga, J. T. Wang, D. Ramage, N. Amin, B. Schwikowski, T. Ideker, Genome Research 2003, 13, 2498.
[A35] M. M. Al-Mathkour, A. M. Dwead, E. Alp, A. M. Boston, B. Cinar, Sci Rep 2022, 12, 3840.
[A36] X. Yang, M. Delsante, P. Daneshpajouhnejad, P. Fenaroli, K. P. Mandell, X. Wang, S. Takahashi, M. K. Halushka, J. B. Kopp, M. Levi, A. Z. Rosenberg, 2021.
[A37] T. Huang, Y. Zhou, J. Zhang, A. S. L. Cheng, J. Yu, K. F. To, W. Kang, Journal of Translational Medicine 2018, 16, 98.
[A38] X. Liu, N. Yang, S. A. Figel, K. E. Wilson, C. D. Morrison, I. H. Gelman, J. Zhang, Oncogene 2013, 32, 1266.
[A39] J. Feng, J. Gou, J. Jia, T. Yi, T. Cui, Z. Li, Onco Targets Ther 2016, 9, 5371.
[A40] J. Bonse, D. O. Wennmann, J. Kremerskothen, T. Weide, U. Michgehl, H. Pavenstädt, B. Vollenbröker, Cell Death Dis 2018, 9, 1.
[A41] A. Reginensi, R. P. Scott, A. Gregorieff, M. Bagherie-Lachidan, C. Chung, D.-S. Lim, T. Pawson, J. Wrana, H. McNeill, PLOS Genetics 2013, 9, e1003380.
[A42] F. Moisy, “Boxcount,” can be found under https://www.mathworks.com/matlabcentral/fileexchange/13063-boxcount, 2022.
[A43] M. S. Roost, L. van Iperen, Y. Ariyurek, H. P. Buermans, W. Arindrarto, H. D. Devalla, R. Passier, C. L. Mummery, F. Carlotti, E. J. P. de Koning, E. W. van Zwet, J. J. Goeman, S. M. Chuva de Sousa Lopes, Stem Cell Reports 2015, 4, 1112.
[A44] M. I. Love, W. Huber, S. Anders, Genome Biology 2014, 15, 1.
[A45] V. Jalili, E. Afgan, Q. Gu, D. Clements, D. Blankenberg, J. Goecks, J. Taylor, A. Nekrutenko, Nucleic Acids Research 2021, 48, W395.
[A46] P. D. Thomas, D. Ebert, A. Muruganujan, T. Mushayahama, L. P. Albou, H. Mi, Protein Science 2022, 31, 8.
[A47] H. Mi, A. Muruganujan, X. Huang, D. Ebert, C. Mills, X. Guo, P. D. Thomas, Nature Protocols 2019, 14, 703.
[A48] N. Kobayashi, P. Mundel, Cell Tissue Res 1998, 291, 163.
[A49] C. Schell, T. B. Huber, J Am Soc Nephrol 2017, 28, 3166.
[A50] M. Ala, European Journal of Pharmacology 2021, 896, 173921.
[A51] X. Jiang, M. Wu, X. Xu, L. Zhang, Y. Huang, Z. Xu, K. He, H. Wang, H. Wang, L. Teng, Mol Med Rep 2019, 20, 3103.
[A52] C. Li, T. Shao, G. Bao, Z. Gao, Y. Zhang, H. Ding, W. Zhang, F. Liu, C. Guo, Am J Transl Res 2019, 11, 6812.
[A53] Y.-L. Ding, S.-F. Sun, G.-L. Zhao, Medicine (Baltimore) 2021, 100, e24561.
[A54] A. Sohail, Q. Sun, H. Zhao, M. M. Bernardo, J.-A. Cho, R. Fridman, Cancer Metastasis Rev 2008, 27, 289.
[A55] A. Ortiz, H. Husi, L. Gonzalez-Lafuente, L. Valiño-Rivas, M. Fresno, A. B. Sanz, W. Mullen, A. Albalat, S. Mezzano, T. Vlahou, H. Mischak, M. D. Sanchez-Niño, J Am Soc Nephrol 2017, 28, 823.
[A56] G. C. Chan, D. G. Eng, J. H. Miner, C. E. Alpers, K. Hudkins, A. Chang, J. W. Pippin, S. J. Shankland, Am J Physiol Renal Physiol 2019, 317, F1680.
[A57] Y. Shen, N. Miao, B. Wang, J. Xu, X. Gan, D. Xu, L. Zhou, H. Xue, W. Zhang, L. Yang, L. Lu, Kidney International 2017, 92, 888.
[A58] Y.-L. Tang, G.-S. Li, D.-M. Li, D. Tang, J.-Z. Huang, H. Feng, R.-Q. He, Z.-G. Huang, Y.-W. Dang, J.-L. Kong, T.-Q. Gan, H.-F. Zhou, J.-J. Zeng, G. Chen, BMC Pulm Med 2022, 22, 300.
[A59] K. Lee, S. Boctor, L. M. C. Barisoni, G. L. Gusella, J Am Soc Nephrol 2015, 26, 888.
[A60] A. Korolj, H.-T. Wu, M. Radisic, Biomaterials 2019, 219, 119363.
[A61] M. Narlis, D. Grote, Y. Gaitan, S. K. Boualia, M. Bouchard, J Am Soc Nephrol 2007, 18, 1121.

1. An apparatus for cultivation of cells, the apparatus comprising:
a first chamber for cultivating cells; and
a surface, supported in the first chamber, for cell cultivation thereon, the surface exhibiting one or more fractal features, each fractal feature comprising out-of-plane fractal patterning providing non-planar microtopology for the surface.

2. The apparatus of claim 1, wherein each fractal feature includes protruding Gaussian, fractal micro-curvatures.

3. The apparatus of claim 1, wherein complexity of each fractal feature is determined by a fractal dimension (Df), wherein the fractal dimension is defined as:
$$Df = \frac{- log N}{log ε}$$
wherein N is a number of measurement units, and
ε is a scaling factor.

4. The apparatus of claim 3, wherein the fractal patterning is derived from a histological section of a biological microenvironment exhibiting the fractal patterning.

5. The apparatus of claim 4, wherein the biological microenvironment is a podocyte microenvironment.

6. The apparatus of claim 5, wherein the fractal patterning mimics glomeruli in the podocyte microenvironment.

7. The apparatus of claim 5, wherein the micro-curvatures are in convoluted capillary shapes.

8. The apparatus of claim 7, wherein the Df of each fractal feature is at least 2.2.

9. The apparatus of claim 8, wherein an average diameter of the fractal patterning of the one or more fractal features is between 140 to 160 μm.

10. The apparatus of claim 8, wherein peaks in the fractal patterning of each of the fractal features is between 5 to 15 μm.

11. The apparatus of claim 4, wherein the biological microenvironment is a bronchial epithelial, a blood vessel, a lung, or a bone marrow microenvironment.

12. A method of viewing confluent branching cell morphology of cells, the method comprising:
sporadically labeling cytoplasm of a portion of the cells with a fluorescent label in vitro;
mixing the labelled cells with another non-fluorescent portion of the cells;
staining the mixed cells to visualize confluent monolayers;
identifying single labelled cells in contact with non-fluorescent cells that are arranged in a monolayer; and
imaging the monolayer.

13. The method of claim 12, wherein the cells are podocytes.

14. The method of claim 12, wherein the labeling of cytoplasms with the fluorescent label is performed by transient adenoviral or stablelentivirus transduction.

15. The method of claim 14, wherein the labelled cells and the non-fluorescent cells are mixed at a ratio of 1:11.

16. The method of claim 15, wherein the mixed cells are stained with rhodamine-wheat germ agglutinin.

17. The method of claim 16, wherein the monolayer is imaged with a super resolution confocal microscope.

18. The method of claim 12, wherein the fluorescent label is green fluorescent protein (GFP).

19. A method for fabricating an apparatus for cultivation of cells, the method comprising:
drawing fractal patterning onto a substrate using photolithography;
generating non-planar microtopology on the substrate according to the fractal patterning;
forming an inverse mold by curing a first polymer over the non-planar microtopology with fractal patterning;
forming a surface for cell cultivation by curing a second polymer using the inverse mold, the surface being formed to exhibit one or more fractal features, each fractal feature comprising out-of-plane fractal patterning providing non-planar microtopology for the surface; and
supporting at least a portion of the surface in a first chamber for cultivating cells.

20. The method of claim 19, wherein generating the non-planar microtopology on the substrate comprises generating protruding Gaussian and fractal micro-curvatures according to the fractal patterning.

21. The method of claim 20, wherein drawing the fractal patterning onto the substrate comprises:
providing a histological section of a biological microenvironment with the fractal patterning; and
templating the biological microenvironment with the fractal patterning onto the substrate using the photolithography.

22. The method of claim 21, wherein the biological microenvironment is a podocyte microenvironment.

23. The method of claim 22, wherein the fractal patterning copied onto the substrate is between 140 to 160 μm in diameter.

24. The method of claim 22, wherein the protruding Gaussian and fractal micro-curvatures are generated with peaks between 5 to 15 μm.

25. The method of claim 19, wherein the second polymer comprises polydimethylsiloxane (PDMS), polystyrene, or poly(octamethylene maleate (anhydride) 1,2,4-butanetricarboxylate) (124-polymer).

26. The method of claim 25, wherein the second polymer is 124-polymer with an inert polymer incorporated therein, the method further comprising leaching out the inert polymer after curing.
